SEQUENCE LISTING (130 sequences)

SEQ ID NO: 1 - DNA, 785 bp, Artificial Sequence
  Synthetic gfpmut3 with RBS construct
aagtcggtgacagataacaggagtaagtaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttgacttatggtgttcaatgcttttcaagatacccagatcatatgaaacggcatgactttttcaagagtgccatgcccgaaggttatgtacaggaaagaactatatttttcaaagatgacgggaactataagacacgtgctgaagtcaagtttgaaggtgatacacttgttaatagaatcgagttaaaaggtattgattttaaagaagatggaaacattcttggacacaagttggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgcagcaaacgacgaaaactacgcttaagtagcttaa

SEQ ID NO: 2 - DNA, 35 bp, Artificial Sequence
  Synthetic BBa_J23117 (P17) construct
ttgacagctagctcagtcctagggattgtgctagc

SEQ ID NO: 3 - DNA, 133 bp, Artificial Sequence
  Synthetic TyrR construct
ccagcgaaaaataatgcaatatcgggtgctgaccggatatctttacgccgaagtgcccgtttttccgtctttgtgtcaatgattgttgacagaaaccttcctgctatccaaatagtgtcatatcatcatatta

SEQ ID NO: 4 - DNA, 134 bp, Artificial Sequence
  Synthetic TyrR construct
cgtcgtttcggtggtgatgcgtaatcatcgctgaacagcgaacacaatctgtaaaataatatatacagccccgatttttaccatcggggctttttttctgtcttttgtactcgtgtactggtacagtgcaatgc

SEQ ID NO: 5 - DNA, 124 bp, Artificial Sequence
  Synthetic TyrR construct
tctgttgctaattgccgttcgctcctgaacatccactcgatcttcgccttcttccggtttattgtgttttaaccacctgcccgtaaacctggagaaccatcgcgtgtttcaaaaagttgacgcc

SEQ ID NO: 6 - DNA, 130 bp, Artificial Sequence
  Synthetic TyrR construct
attgttttcaaagggagtgtaaatttatctatacagaggtaagggttgaaagcgcgactaaattgcctgtgtaaataaaaatgtacgaaatatggattgaaaactttactttatgtgttatcgttacgtc

SEQ ID NO: 7 - DNA, 154 bp, Artificial Sequence
  Synthetic TyrR construct
cgtccctcctcaaaaaaagcctagcgtagcgattgccgcttatgaagactttgcgccagcgcaggactgaatgctttttattgtacatttatatttacaccatatgtaacgtcggtttgacgaagcagccgttatgccttaacctgcgccgcag

SEQ ID NO: 8 - DNA, 150 bp, Artificial Sequence
  Synthetic TyrR construct
aagcaactcatcttcaaccatgcataaagcgggtgcattcgctgccgcataccattattcttgatctgacggaagtctttttgtaacaattcaaacttctttgatgtaaacaaattaatacaacaaacggaattgcaaacttacacacgc

SEQ ID NO: 9 - DNA, 130 bp, Artificial Sequence
  Synthetic TyrR construct
attgttttcaaagggagtgtaaatttatctatacagaggtaagggttgaaagcgcgactaaattgcctgtgtaaataaaaatgtacgaaatatggattgaaaactttactttatgaggtataatgctagc

SEQ ID NO: 10 - DNA, 149 bp, Artificial Sequence
  Synthetic TyrR construct
aagcaactcatcttcaaccatgcataaagcgggtgcattcgctgccgcataccattattcttgacagctagctcagtcctaggtataatgctagcttctttgatgtaaacaaattaatacaacaaacggaattgcaaacttacacacgc

SEQ ID NO: 11 - DNA, 1542 bp, Artificial Sequence
  Synthetic TyrR construct
atgcgtctggaagtcttttgtgaagaccgactcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 12 - DNA, 1542 bp, Artificial Sequence
  Synthetic TyrR construct
atgcgtctggaagtcttttgtgaagaccgactcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtcaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 13 - DNA, 1542 bp, Artificial Sequence
  Synthetic TyrR construct
atgcgtctggaagtcttttgtgaagaccgagggggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtcaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 14 - DNA, 1542 bp, Artificial Sequence; identical to SEQ ID NO: 12
  Synthetic TyrR construct

SEQ ID NO: 15 - DNA, 1542 bp, Artificial Sequence
  Synthetic TyrR construct
atgcgtctggaagtcttttgtgaagaccgactcggtctggggcgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtcaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 16 - DNA, 1542 bp, Artificial Sequence
  Synthetic TyrR construct
atgcgtctggaagtcttttgtgaagaccgactcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtgcacatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtcaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 17 - DNA, 1542 bp, Artificial Sequence
  Synthetic TyrR construct
atgcgtctggaagtcttttgtgaagaccgactcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtgtccatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtcaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 18 - DNA, 1542 bp, Artificial Sequence
  Synthetic TyrR construct
atgcgtctggaagtctttgttgaagaccgactcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtcaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 19 - DNA, 1542 bp, Artificial Sequence
  Synthetic TyrR construct
atgcgtctggaagtcttttgtgaagactttctcggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 20 - DNA, 1542 bp, Artificial Sequence
  Synthetic TyrR construct
atgcgtctggaagtcttttgtgaagaccgagggggtctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 21 - DNA, 1542 bp, Artificial Sequence
  Synthetic TyrR construct
atgcgtctggaagtcttttgtgaagaccgactcacgctgacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 22 - DNA, 1542 bp, Artificial Sequence
  Synthetic TyrR construct
atgcgtctggaagtcttttgtgaagaccgactcggtggaacccgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 23 - DNA, 1542 bp, Artificial Sequence
  Synthetic TyrR construct
atgcgtctggaagtcttttgtgaagaccgactcggtctgaagcgcgaattactcgatctactcgtgctaagaggcattgatttacgcggtattgagattgatcccattgggcgaatctacctcaattttgctgaactggagtttgagagtttcagcagtctgatggccgaaatacgccgtattgcgggtgttaccgatgtgcgtactgtcccgtggatgccttccgaacgtgagcatctggcgttgagcgcgttactggaggcgttgcctgaacctgtgctctctgtcgatatgaaaagcaaagtggatatggcgaacccggcgagctgtcagctttttgggcaaaaattggatcgcctgcgcaaccataccgccgcacaattgattaacggctttaattttttacgttggctggaaagcgaaccgcaagattcgcataacgagcatgtcgttattaatgggcagaatttcctgatggagattacgcctgtttatcttcaggatgaaaatgatcaacacgtcctgaccggtgcggtggtgatgttgcgatcaacgattcgtatgggccgccagttgcaaaatgtcgccgcccaggacgtcagcgccttcagtcaaattgtcgccgtcagcccgaaaatgaagcatgttgtcgaacaggcgcagaaactggcgatgctaagcgcgccgctgctgattacgggtgacacaggtacaggtaaagatctctttgcctacgcctgccatcaggcaagccccagagcgggcaaaccttacctggcgctgaactgtgcgtctataccggaagatgcggtcgagagtgaactgtttggtcatgctccggaagggaagaaaggattctttgagcaggcgaacggtggttcggtgctgttggatgaaataggggaaatgtcaccacggatgcaggcgaaattactgcgtttccttaatgatggcactttccgtcgggttggcgaagaccatgaggtgcatgtcgatgtgcgggtgatttgcgctacgcagaagaatctggtcgaactggtgcaaaaaggcatgttccgtgaagatctctattatcgtctgaacgtgttgacgctcaatctgccgccgctacgtgactgtccgcaggacatcatgccgttaactgagctgttcgtcgcccgctttgccgacgagcagggcgtgccgcgtccgaaactggccgctgacctgaatactgtacttacgcgttatgcgtggccgggaaatgtgcggcagttaaagaacgctatctatcgcgcactgacacaactggacggttatgagctgcgtccacaggatattttgttgccggattatgacgccgcaacggtagccgtgggcgaagatgcgatggaaggttcgctggacgaaatcaccagccgttttgaacgctcggtattaacccagctttatcgcaattatcccagcacgcgcaaactggcaaaacgtctcggcgtttcacataccgcgattgccaataagttgcgggaatatggtctgagtcagaagaagaacgaagagtaa

SEQ ID NO: 24 - DNA, 247 bp, Artificial Sequence
  Synthetic TynA-FeaR construct
tcggtgaatgaagggcaacggcgaatagttgcccttttatttcactaagttttgtgacgttgtcacattatgcatgatgtgtacatctattttcagggcatccactgtatgaaaagctggcacacctgccaaacaacctggcaggtgcaggcaatcccctttgcatcagtactgataatgtgaacctgactaaaccgcccacagagcgcggttgctaacaagaacacaacagaaagaggggttaata

SEQ ID NO: 25 - DNA, 140 bp, Artificial Sequence
  Synthetic TynA-FeaR construct
tatgaaaagctggcacacctgccaaacaacctggcaggtgcaggcaatcccctttgcatcagtactgataatgtgaacctgactaaaccgcccacagagcgcggttgctaacaagaacacaacagaaagaggggttaata

SEQ ID NO: 26 - DNA, 30 bp, Artificial Sequence
  Synthetic TynA-FeaR construct
agaacacaacarrrrrrrrrgttaataatg

SEQ ID NO: 27 - DNA, 29 bp, Artificial Sequence
  Synthetic TynA-FeaR construct
agaacacaacagaaagagggttaataatg

SEQ ID NO: 28 - DNA, 29 bp, Artificial Sequence
  Synthetic TynA-FeaR construct
aactaagacgwtctrggbgtcawhaaatg

SEQ ID NO: 29 - DNA, 887 bp, Artificial Sequence
  Synthetic TynA-FeaR construct
aactaagacgttctaggcgtcatcaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 30 - DNA, 27 bp, Artificial Sequence
  Synthetic TynA-FeaR construct
caaaatccwcaadggtrgttgbbgatg

SEQ ID NO: 31 - DNA, 1446 bp, Artificial Sequence
  Synthetic TynA-FeaR construct
caaaatcctcaaaggtggttgcggatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtatctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataa

SEQ ID NO: 32 - DNA, 2274 bp, Artificial Sequence
  Synthetic TynA-FeaR construct
atgggaagcccctctctgtattctgcccgtaaaacaaccctggcgttggcagtcgccttaagtttcgcctggcaagcgccggtatttgcccacggtggtgaagcgcatatggtgccaatggataaaacgcttaaagaatttggtgccgatgtgcagtgggacgactacgcccagctctttaccctgattaaagatggcgcgtacgtgaaagtgaagcctggtgcgcaaacagcaattgttaatggtcagcctctggcactgcaagtaccggtagtgatgaaagacaataaagcctgggtttctgacacctttattaacgatgttttccagtccgggctggatcaaacctttcaggtagaaaagcgccctcacccacttaatgcgctaactgcggacgaaattaaacaggccgttgaaattgttaaagcttccgcggacttcaaacccaatacccgttttactgagatctccctgctaccgccagataaagaagctgtctgggcgtttgcgctggaaaacaaaccggttgaccagccgcgcaaagccgacgtcattatgctcgacggcaaacatatcatcgaagcggtggtggatctgcaaaacaacaaactgctctcctggcaacccattaaagacgcccacggtatggtgttgctggatgatttcgccagtgtgcagaacattattaacaacagtgaagaatttgccgctgccgtgaagaaacgcggtattactgatgcgaaaaaagtgattaccacgccgctgaccgtaggttatttcgatggtaaagatggcctgaaacaagatgcccggttgctcaaagtcatcagctatcttgatgtcggtgatggcaactactgggcacatcccatcgaaaacctggtggcggtcgttgatttagaacagaaaaaaatcgttaagattgaagaaggtccggtagttccggtgccaatgaccgcacgcccatttgatggccgtgaccgcgttgctccggcagttaagcctatgcaaatcattgagcctgaaggtaaaaattacaccattactggcgatatgattcactggcggaactgggattttcacctcagcatgaactctcgcgtcgggccgatgatctccaccgtgacttataacgacaatggcaccaaacgcaaagtcatgtacgaaggttctctcggcggcatgattgtgccttacggtgatcctgatattggctggtactttaaagcgtatctggactctggtgactacggtatgggcacgctaacctcaccaattgctcgtggtaaagatgccccgtctaacgcagtgctccttaatgaaaccatcgccgactacactggcgtgccgatggagatccctcgcgctatcgcggtatttgaacgttatgccgggccggagtataagcatcaggaaatgggccagcccaacgtcagtaccgaacgccgggagttagtggtgcgctggatcagtacagtgggtaactatgactacatttttgactggatcttccatgaaaacggcactattggcatcgatgccggtgctacgggcatcgaagcggtgaaaggtgttaaagcgaaaaccatgcacgatgagacggcgaaagatgacacgcgctacggcacgcttatcgatcacaatatcgtgggtactacacaccaacatatttataatttccgcctcgatctggatgtagatggcgagaataacagcctggtggcgatggacccagtggtaaaaccgaatactgccggtggcccacgcaccagtaccatgcaagttaatcagtacaacatcggcaatgaacaggatgccgcacagaaatttgatccgggcacgattcgtctgttgagtaacccgaacaaagagaaccgcatgggcaatccggtttcctatcaaattattccttatgcaggtggtactcacccggtagcaaaaggtgcccagttcgcgccggacgagtggatctatcatcgtttaagctttatggacaagcagctctgggtaacgcgttatcatcctggcgagcgtttcccggaaggcaaatatccgaaccgttctactcatgacaccggtcttggacaatacagtaaggataacgagtcgctggacaacaccgacgccgttgtctggatgaccaccggcaccacacatgtggcccgcgccgaagagtggccgattatgccgaccgaatgggtacatactctgctgaaaccatggaacttctttgacgaaacgccaacgctaggggcgctgaagaaagataagtga

SEQ ID NO: 33 - DNA, 2274 bp, Artificial Sequence
  Synthetic TynA-FeaR construct
atgggaagcccctctctgtattctgcccgtaaaacaaccctggcgttggcagtcgccttaagtttcgcctggcaagcgccggtatttgcccacggtggtgaagcgcatatggtgccaatggataaaacgcttaaagaatttggtgccgatgtgcagtgggacgactacgcccagctctttaccctgattaaagatggcgcgtacgtgaaagtgaagcctggtgcgcaaacagcaattgttaatggtcagcctctggcactgcaagtaccggtagtgatgaaagacaataaagcctgggtttctgacacctttattaacgatgttttccagtccgggctggatcaaacctttcaggtagaaaagcgccctcacccacttaatgcgctaactgcggacgaaattaaacaggccgttgaaattgttaaagcttccgcggacttcaaacccaatacccgttttactgagatctccctgctaccgccagataaagaagctgtctgggcgtttgcgctggaaaacaaaccggttgaccagccgcgcaaagccgacgtcattatgctcgacggcaaacatatcatcgaagcggtggtggatctgcaaaacaacaaactgctctcctggcaacccattaaagacgcccacggtatggtgttgctggatgatttcgccagtgtgcagaacattattaacaacagtgaagaatttgccgctgccgtgaagaaacgcggtattactgatgcgaaaaaagtgattaccacgccgctgaccgtaggttatttcgatggtaaagatggcctgaaacaagatgcccggttgctcaaagtcatcagctatcttgatgtcggtgatggcaactactgggcacatcccatcgaaaacctggtggcggtcgttgatttagaacagaaaaaaatcgttaagattgaagaaggtccggtagttccggtgccaatgaccgcacgcccatttgatggccgtgaccgcgttgctccggcagttaagcctatgcaaatcattgagcctgaaggtaaaaattacaccattactggcgatatgattcactggcggaactgggattttcacctcagcatgaactctcgcgtcgggccgatgatctccaccgtgacttataacgacaatggcaccaaacgcaaagtcatgtacgaaggttctctcggcggcatgattgtgccttacggtgatcctgatattggctggtactttaaagcgtatctggactctggtgactacggtatgggcacgctaacctcaccaattgctcgtggtaaagatgccccgtctaacgcagtgctccttaatgaaaccatcgccgactacactggcgtgccgatggagatccctcgcgctatcgcggtatttgaacgttatgccgggccggagtataagcatcaggaaatgggccagcccaacgtcagtaccgaacgccgggagttagtggtgcgctggatcagtacagtgtccaactatgactacatttttgactggatcttccatgaaaacggcactattggcatcgatgccggtgctacgggcatcgaagcggtgaaaggtgttaaagcgaaaaccatgcacgatgagacggcgaaagatgacacgcgctacggcacgcttatcgatcacaatatcgtgggtactacacaccaacatatttataatttccgcctcgatctggatgtagatggcgagaataacagcctggtggcgatggacccagtggtaaaaccgaatactgccggtggcccacgcaccagtaccatgcaagttaatcagtacaacatcggcaatgaacaggatgccgcacagaaatttgatccgggcacgattcgtctgttgagtaacccgaacaaagagaaccgcatgggcaatccggtttcctatcaaattattccttatgcaggtggtactcacccggtagcaaaaggtgcccagttcgcgccggacgagtggatctatcatcgtttaagctttatggacaagcagctctgggtaacgcgttatcatcctggcgagcgtttcccggaaggcaaatatccgaaccgttctactcatgacaccggtcttggacaatacagtaaggataacgagtcgctggacaacaccgacgccgttgtctggatgaccaccggcaccacacatgtggcccgcgccgaagagtggccgattatgccgaccgaatgggtacatactctgctgaaaccatggaacttctttgacgaaacgccaacgctaggggcgctgaagaaagataagtga

SEQ ID NO: 34 - DNA, 2274 bp, Artificial Sequence
  Synthetic TynA-FeaR construct
atgggaagcccctctctgtattctgcccgtaaaacaaccctggcgttggcagtcgccttaagtttcgcctggcaagcgccggtatttgcccacggtggtgaagcgcatatggtgccaatggataaaacgcttaaagaatttggtgccgatgtgcagtgggacgactacgcccagctctttaccctgattaaagatggcgcgtacgtgaaagtgaagcctggtgcgcaaacagcaattgttaatggtcagcctctggcactgcaagtaccggtagtgatgaaagacaataaagcctgggtttctgacacctttattaacgatgttttccagtccgggctggatcaaacctttcaggtagaaaagcgccctcacccacttaatgcgctaactgcggacgaaattaaacaggccgttgaaattgttaaagcttccgcggacttcaaacccaatacccgttttactgagatctccctgctaccgccagataaagaagctgtctgggcgtttgcgctggaaaacaaaccggttgaccagccgcgcaaagccgacgtcattatgctcgacggcaaacatatcatcgaagcggtggtggatctgcaaaacaacaaactgctctcctggcaacccattaaagacgcccacggtatggtgttgctggatgatttcgccagtgtgcagaacattattaacaacagtgaagaatttgccgctgccgtgaagaaacgcggtattactgatgcgaaaaaagtgattaccacgccgctgaccgtaggttatttcgatggtaaagatggcctgaaacaagatgcccggttgctcaaagtcatcagctatcttgatgtcggtgatggcaactactgggcacatcccatcgaaaacctggtggcggtcgttgatttagaacagaaaaaaatcgttaagattgaagaaggtccggtagttccggtgccaatgaccgcacgcccatttgatggccgtgaccgcgttgctccggcagttaagcctatgcaaatcattgagcctgaaggtaaaaattacaccattactggcgatatgattcactggcggaactgggattttcacctcagcatgaactctcgcgtcgggccgatgatctccaccgtgacttataacgacaatggcaccaaacgcaaagtcatgtacgaaggttctctcggcggcatgattgtgccttacggtgatcctgatattggctggtactttaaagcgtatctggactctcacgactacggtatgggcacgctaacctcaccaattgctcgtggtaaagatgccccgtctaacgcagtgctccttaatgaaaccatcgccgactacactggcgtgccgatggagatccctcgcgctatcgcggtatttgaacgttatgccgggccggagtataagcatcaggaaatgggccagcccaacgtcagtaccgaacgccgggagttagtggtgcgctggatcagtacagtgggtaactatgactacatttttgactggatcttccatgaaaacggcactattggcatcgatgccggtgctacgggcatcgaagcggtgaaaggtgttaaagcgaaaaccatgcacgatgagacggcgaaagatgacacgcgctacggcacgcttatcgatcacaatatcgtgggtactacacaccaacatatttataatttccgcctcgatctggatgtagatggcgagaataacagcctggtggcgatggacccagtggtaaaaccgaatactgccggtggcccacgcaccagtaccatgcaagttaatcagtacaacatcggcaatgaacaggatgccgcacagaaatttgatccgggcacgattcgtctgttgagtaacccgaacaaagagaaccgcatgggcaatccggtttcctatcaaattattccttatgcaggtggtactcacccggtagcaaaaggtgcccagttcgcgccggacgagtggatctatcatcgtttaagctttatggacaagcagctctgggtaacgcgttatcatcctggcgagcgtttcccggaaggcaaatatccgaaccgttctactcatgacaccggtcttggacaatacagtaaggataacgagtcgctggacaacaccgacgccgttgtctggatgaccaccggcaccacacatgtggcccgcgccgaagagtggccgattatgccgaccgaatgggtacatactctgctgaaaccatggaacttctttgacgaaacgccaacgctaggggcgctgaagaaagataagtga

SEQ ID NO: 35 - DNA, 2274 bp, Artificial Sequence
  Synthetic TynA-FeaR construct
atgggaagcccctctctgtattctgcccgtaaaacaaccctggcgttggcagtcgccttaagtttcgcctggcaagcgccggtatttgcccacggtggtgaagcgcatatggtgccaatggataaaacgcttaaagaatttggtgccgatgtgcagtgggacgactacgcccagctctttaccctgattaaagatggcgcgtacgtgaaagtgaagcctggtgcgcaaacagcaattgttaatggtcagcctctggcactgcaagtaccggtagtgatgaaagacaataaagcctgggtttctgacacctttattaacgatgttttccagtccgggctggatcaaacctttcaggtagaaaagcgccctcacccacttaatgcgctaactgcggacgaaattaaacaggccgttgaaattgttaaagcttccgcggacttcaaacccaatacccgttttactgagatctccctgctaccgccagataaagaagctgtctgggcgtttgcgctggaaaacaaaccggttgaccagccgcgcaaagccgacgtcattatgctcgacggcaaacatatcatcgaagcggtggtggatctgcaaaacaacaaactgctctcctggcaacccattaaagacgcccacggtatggtgttgctggatgatttcgccagtgtgcagaacattattaacaacagtgaagaatttgccgctgccgtgaagaaacgcggtattactgatgcgaaaaaagtgattaccacgccgctgaccgtaggttatttcgatggtaaagatggcctgaaacaagatgcccggttgctcaaagtcatcagctatcttgatgtcggtgatggcaactactgggcacatcccatcgaaaacctggtggcggtcgttgatttagaacagaaaaaaatcgttaagattgaagaaggtccggtagttccggtgccaatgaccgcacgcccatttgatggccgtgaccgcgttgctccggcagttaagcctatgcaaatcattgagcctgaaggtaaaaattacaccattactggcgatatgattcactggcggaactgggattttcacctcagcatgaactctcgcgtcgggccgatgatctccaccgtgacttataacgacaatggcaccaaacgcaaagtcatgtacgaaggttctctcggcggcatgattgtgccttacggtgatcctgatattggctggtactttaaagcgtatctggacatgggtgactacggtatgggcacgctaacctcaccaattgctcgtggtaaagatgccccgtctaacgcagtgctccttaatgaaaccatcgccgactacactggcgtgccgatggagatccctcgcgctatcgcggtatttgaacgttatgccgggccggagtataagcatcaggaaatgggccagcccaacgtcagtaccgaacgccgggagttagtggtgcgctggatcagtacagtgggtaactatgactacatttttgactggatcttccatgaaaacggcactattggcatcgatgccggtgctacgggcatcgaagcggtgaaaggtgttaaagcgaaaaccatgcacgatgagacggcgaaagatgacacgcgctacggcacgcttatcgatcacaatatcgtgggtactacacaccaacatatttataatttccgcctcgatctggatgtagatggcgagaataacagcctggtggcgatggacccagtggtaaaaccgaatactgccggtggcccacgcaccagtaccatgcaagttaatcagtacaacatcggcaatgaacaggatgccgcacagaaatttgatccgggcacgattcgtctgttgagtaacccgaacaaagagaaccgcatgggcaatccggtttcctatcaaattattccttatgcaggtggtactcacccggtagcaaaaggtgcccagttcgcgccggacgagtggatctatcatcgtttaagctttatggacaagcagctctgggtaacgcgttatcatcctggcgagcgtttcccggaaggcaaatatccgaaccgttctactcatgacaccggtcttggacaatacagtaaggataacgagtcgctggacaacaccgacgccgttgtctggatgaccaccggcaccacacatgtggcccgcgccgaagagtggccgattatgccgaccgaatgggtacatactctgctgaaaccatggaacttctttgacgaaacgccaacgctaggggcgctgaagaaagataagtga

SEQ ID NO: 36 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 37 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagacaataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 38 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagttgataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 39 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagcccataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 40 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagtccataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 41 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggtgataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 42 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggagataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 43 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagatgataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 44 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagggcataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 45 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagatcataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 46 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagttcataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 47 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagtacataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 48 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagtggataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 49 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagtgcataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 50 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagaacataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 51 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagcagataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 52 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagaagataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 53 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagcacataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 54 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcagcgcataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 55 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggacataatggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 56 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataggcgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 57 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatagcggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 58 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatacccgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 59 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatagttgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 60 - DNA, 906 bp, Artificial Sequence; identical to SEQ ID NO: 59
  Synthetic TynA-FeaR construct

SEQ ID NO: 61 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatattggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 62 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatattcgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 63 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatatacgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 64 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatatgggagcaggatgagcgtcaggtgcagattggcgctggcgatattacgttactcgatgcctcacgcccctgttcgtggtactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 65 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatatccgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 66 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatatgcgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 67 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataacggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 68 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataaacgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 69 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatacaagagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 70 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatacacgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 71 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataaaggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 72 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatacgcgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 73 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatagatgagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 74 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaatagaggagcaggatgagcgtcaggtgcagattggcgctggcgatattacgttactcgatgcctcacgcccctgttcgctttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 75 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcggggtactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 76 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcggcgtactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 77 - DNA, 906 bp, Artificial Sequence; identical to SEQ ID NO: 76
  Synthetic TynA-FeaR construct

SEQ ID NO: 78 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcggtctactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 79 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgacgtactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 80 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgatctactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 81 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgttttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 82 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgtactactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 83 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgtggtactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 84 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgtcctactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 85 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgtgctactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 86 - DNA, 906 bp, Artificial Sequence; identical to SEQ ID NO: 79
  Synthetic TynA-FeaR construct

SEQ ID NO: 87 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgaactactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 88 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgcagtactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 89 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgcactactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 90 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgaaatactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 91 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgcgttactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 92 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcggactactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 93 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcggagtactggcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 94 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacggccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 95 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacgcgcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 96 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacccgcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 97 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacgtgcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 98 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacatgcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 99 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacatacaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 100 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacctccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 101 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacttccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 102 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttactaccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 103 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttactcccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 104 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttactgtcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 105 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacacccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 106 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacaaccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 107 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttaccagcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 108 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttaccaccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 109 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacaagcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 110 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttaccgccaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 111 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacgatcaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 112 - DNA, 906 bp, Artificial Sequence
  Synthetic TynA-FeaR construct
atgaaccccgcagtggataatgagtttcagcaatggctttcccaaatcaatcaggtatgcggaaattttaccggacgcctgctgactgagcgttacacgggtgtactggacacgcattttgccaaaggactaaagctgagtaccgtgacaaccagcggggtgaatttatcccgcacctggcaggaagtaaaaggcagcgacgatgcctggttttacaccgtttttcagcttagtggtcaggcaataatggagcaggatgagcgtcaggtgcagattggcgctggcgatattacgttactcgatgcctcacgcccctgttcgctttacgaacaggagtcttctaaacagatttcattacttttgccacgcactctgctggaacaatattttccccatcaaaaacctatctgcgcagaaagactggacgctgacttacccatggtgcaactcagtcatcgcctgttacaggagagcatgaataatccggcactttctgaaacagaaagtgaagctgcgctacaggcgatggtgtgtctgctgcgcccggtacttcatcagcgggaatctgttcaacctcgtcgtgaacgtcagtttcaaaaagtggttacgttgatagacgataatattcgcgaagagatattacgcccggagtggatagccggagagacaggtatgtcagtacgtagtttgtaccgaatgtttgccgataaaggtttggtagtcgcgcaatatattcgtaaccgtcgtctcgatttttgtgcagatgcgattcgccatgccgcagatgatgaaaaactggcaggcatcggctttcattggggattttctgaccagagtcatttttcaacggtatttaagcaacgctttgggatgacgccaggcgagtatcgacgtaaattccgctaa

SEQ ID NO: 113 - DNA, 327 bp, Escherichia coli
atggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgattga

SEQ ID NO: 114 - DNA, 327 bp, Artificial Sequence
  Synthetic TrpR construct
atggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaagcaatgaactcggcgcatgctgcctgacgattcgccgtggatctaacagcctgaaagccgcgcccgtcgagctgcgccagtggctggaagaggtgttgctgaaaagcgattga

SEQ ID NO: 115 - DNA, 327 bp, Artificial Sequence
  Synthetic TrpR construct
atggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaagcaatgaactcggcgcaaactggcgcacgattaagcgtggatctaacagcctgaaagccgcgcccgtcgagctgcgccagtggctggaagaggtgttgctgaaaagcgattga

SEQ ID NO: 116 - DNA, 374 bp, Artificial Sequence
  Synthetic TrpR construct
aaccgggggaggcattttgcttcccccgctaacaatggcgacatattatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgattga

SEQ ID NO: 117 - DNA, 374 bp, Artificial Sequence
  Synthetic TrpR construct
aaccgggggaggcattttgcttcccctgctaacaaaggagacattttatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgattga

SEQ ID NO: 118 - DNA, 374 bp, Artificial Sequence
  Synthetic TrpR construct
aaccgggggaggcattttgcttcccccgctaacaatggcgacatattatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaagcaatgaactcggcgcaaactggcgcacgattaagcgtggatctaacagcctgaaagccgcgcccgtcgagctgcgccagtggctggaagaggtgttgctgaaaagcgattga

SEQ ID NO: 119 - DNA, 42 bp, Escherichia coli
gagctgttgacaattaatcatcgaactagttaactagtacgc

SEQ ID NO: 120 - DNA, 42 bp, Artificial Sequence
  Synthetic TrpR construct
gagctgttgacaattaatcatcgagtaagttaacttacacgc

SEQ ID NO: 121 - DNA, 42 bp, Artificial Sequence
  Synthetic TrpR construct
gagctgttgacaattaatcatcgatgtagttaactacaacgc

SEQ ID NO: 122 - protein, 108 aa, Escherichia coli
MAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 123 - protein, 757 aa, Escherichia coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK

SEQ ID NO: 124 - protein, 513 aa, Escherichia coli
MRLEVFCEDRLGLTRELLDLLVLRGIDLRGIEIDPIGRIYLNFAELEFESFSSLMAEIRRIAGVTDVRTVPWMPSEREHLALSALLEALPEPVLSVDMKSKVDMANPASCQLFGQKLDRLRNHTAAQLINGFNFLRWLESEPQDSHNEHVVINGQNFLMEITPVYLQDENDQHVLTGAVVMLRSTIRMGRQLQNVAAQDVSAFSQIVAVSPKMKHVVEQAQKLAMLSAPLLITGDTGTGKDLFAYACHQASPRAGKPYLALNCASIPEDAVESELFGHAPEGKKGFFEQANGGSVLLDEIGEMSPRMQAKLLRFLNDGTFRRVGEDHEVHVDVRVICATQKNLVELVQKGMFREDLYYRLNVLTLNLPPLRDCPQDIMPLTELFVARFADEQGVPRPKLAADLNTVLTRYAWPGNVRQLKNAIYRALTQLDGYELRPQDILLPDYDAATVAVGEDAMEGSLDEITSRFERSVLTQLYRNYPSTRKLAKRLGVSHTAIANKLREYGLSQKKNEE

SEQ ID NO: 125 - protein, 301 aa, Escherichia coli
MNPAVDNEFQQWLSQINQVCGNFTGRLLTERYTGVLETHFAKGLKLSTVTTNGVNLYRTWQEIKGSDDAWFYTVFQLSGQAIMEQDERQVQIGAGDITLLDASRPCSLYWQESSKQISLLLPRTLLEQYFPHQKPVCAERLDADLPMVQLSHRLLQESMNNPALSETESEAALQAMVCLLRPVLHQRESVQPRRERQFQKVVTLIDDNIREEILRPEWIAGETGMSVRSLYRMFADKGLVVAQYIRNRRLDFCADAIRHAADDEKLAGIGFHWGFSDQSHFSTVFKQRFGMTPGEYRRKFR

SEQ ID NO: 126 - DNA, 2268 bp, Klebsiella aerogenes
atggcaaacggcttgatgttttcccctcgtaaaaccgcgctggcgctggctgtcgcggtggtttgcgcctggcaatcaccggtcttcgctcacggtagcgaagcgcacatggtgccgttggataaaacgctgcaggcgttcggcgccgatgtgcagtgggatgactacgcgcaaatgttcaccctgataaaagacggcgcttacgtcaaagtaaaacccggcgccaaaacggcgatcgtcaacggtaaaccccttgatctacaggtgccggtagtaatgaaggaaggtaaagcctgggtctccgatacctttatcaacgatgtattccagtccggtctcgatcagaccttccaggtagaaaaacgccctcacccgttaaattcgctctcggcggcggaaatcggtgaagcagtgaccattgttaaagccgcgccggagttccagccgaatacccgctttactgagatttccttacacgagccggataaagcggcggtatgggcctttgccctgcagggaacacccgttgatgccccccgcaccgccgatgtggtgatgctcgatggcaaacatgtcattgaagccgtcgtcgatctgcaaaacaaaaaaatcctctcatggacgccgattaaaggcgcccacgggatggtcttgcttgatgacttcgtcagcgtgcagaacattatcaatgccagcagcgagttcgctgaggtgctgaaaaagcacggtattaccgaccccagtaaggtggtcaccaccccgctcaccgtcggctactttgatggcaaagatggcctgcagcaggatgcgcgtctgctgaaagtcgtcagttatctggataccggcgacggcaactactgggcgcacccgattgaaaacctggtggcggtggtcgaccttgaagcgaagaaaatcatcaaaatcgaagaaggcccggtgatcccggtaccgatggagccacgtccgtatgatggtcgcgaccgcaacgccccggcggtgaaaccgctggagataaccgaaccggaaggcaaaaactacacgatcaccggcgataccattcactggcggaactgggattttcatctgcgcctgaactcgcgcgtcgggcccattctctcgacggtgacttacaacgacaacggcacaaaacgccaggtgatgtatgaaggttccctcggcgggatgatcgtcccctacggcgacccggacgtcggctggtatttcaaagcctatctggactccggcgattacggcatgggcaccctgacgtcgccgattgttcgcggtaaagatgcgccgtcaaatgcggtactgctggacgaaactatcgccgattacaccggcaaaccgaccactattccaggcgcggtcgccatattcgaacgctatgccgggcctgagtataagcacctggaaatgggcaaacccaacgtcagcaccgaacgccgggaactggtggtgcgctggatcagtaccgtcgggaactatgattatatctttgactgggtgttccatgacaacggcaccatcggtatcgatgccggcgccaccggcattgaagccgttaaaggcgtgaaggcgaagaccatgcacgaccccagcgccaaagaggatacccgctacgggacgctaatcgaccataatattgtcggcaccacccaccagcatatttataatttccgcctcgatctcgacgtggacggcgaaaacaacaccctggtggcgatggatcctgaagtgaagccaaacaccgccggcggcccgcgcaccagcaccatgcaggtgaatcagtacacaatcgatagcgagcagaaagcggcgcagaaattcgaccctggcactatccgcctgctgagcaacaccagcaaagagaaccgcatgggtaacccggtctcttaccagattatcccttatgccggcggcacgcatccggcggcgaccggcgccaagttcgccccggacgagtggatatatcatcgcctgagctttatggataaacagctgtgggtgacgcgttaccacccgacagagcgttatccggaagggaaatatcccaaccgttccgcccaggataccggcttaggccagtacgcgaaggatgatgagtcgctgactaaccacgacgacgtcgtgtggatcaccaccggcaccacccacgtcgcgcgcgccgaagagtggccaattatgccgaccgagtgggcgcatgcgctgctcaaaccgtggaacttctttgacgaaaccccaacgcttggcgagaagaaaaagtaa

SEQ ID NO: 127 - DNA, 2268 bp, Klebsiella pneumoniae
atggcaaatggactgaaattcagccctcgtaaaacagctcttgccctggccgttgccgtcgtttgtgcttggcaatcccctgtgtttgctcacggttccgaggcacatatggtacccttggacaaaacattacaggagtttggggccgacgttcagtgggacgactacgcacagatgtttactcttatcaaggacggagcttatgtgaaagtcaagcctggagcaaaaactgcaattgtaaatggaaagtctttagatcttcccgtcccggttgtgatgaaggaaggcaaagcctgggttagcgacacttttatcaatgatgtgtttcaatccggccttgatcagacctttcaggtggaaaagcgtcctcatcccttgaatagtttgagtgcagccgagatctctgaggctgttacaatcgttaaggcagctccggaatttcaaccaaatacccgctttacagagatttcattgcacgaaccggacaaggctgcggtctgggcttttgccctgcaaggtacgccagttgatgcaccgcgtaccgcggacgtcgtcatgttagacggtaagcatgttatcgaagcagtagtagacttgcagaataaaaaaattctttcgtggactccgattaagggggcccacggtatggtcctgttagacgatttcgtctccgtacaaaacattatcaatgcctcatccgagtttgcagaggtcttgaaaaaacacggaattactgacccggggaaagtcgtgacaactcccctgactgtaggtttttttgatggcaaggatggattgcaacaagacgcacgcctgttaaaagtcgtctcgtatctggatacgggtgatggtaattactgggcgcaccctatcgagaacttagtggccgtagttgacctggaggccaagaaaattatcaaaatcgaagaaggtccagtgattcccgtaccgatggagccccgtccatacgatgggcgcgaccgcaatgccccagctgtcaaacctctggaaatcacagagcctgaagggaaaaattatactattactggtgatactatccactggcagaattgggacttccatctgcgtcttaatagtcgcgtgggacccattttatcaacagtcacttacaatgacaatgggacaaagcgtcaggtgatgtatgagggtagtcttggggggatgattgtcccctacggtgatcctgatgtagggtggtatttcaaagcttatcttgactccggcgattacggcatgggaacgcttacgtcgcctattgttcgtggcaaagatgctccctcgaatgccgttcttttggatgagacgatcgctgattacacaggcaagcctactacgattccgggggcggtggcgatttttgaacgttacgcaggacctgagtacaaacaccttgagatgggaaagcccaatgtcagcactgaacgccgtgagcttgtcgtacgttggatctcaaccgttggtaattatgactatattttcgactgggtatttcacgacaatggaacgattggcatcgatgctggggctacagggatcgaggcagtgaaaggagtgttggcaaaaactatgcatgacccttcggctaaagaggatacacgctacggcactctgattgaccacaacattgtgggtacaacgcatcagcacatctataactttcgtttggaccttgacgttgatggggagaataacacgcttgtcgcgatggacccggaagttaagccgaatactgcgggtggtccgcgcacgtcaactatgcaagttaaccagtacacgatcgacagcgagcaaaaggcggctcaaaaattcgatccaggaacaattcgtcttttatctaatacctcgaaggaaaaccgtatgggcaacccggtgtcctaccagatcatcccatatgcaggcggtactcaccctgccgctaccggagccaagtttgcgcccgacgagtggatttatcaccgcctgtcctttatggacaaacaattatgggtaacccgttaccacccgactgaacgctatcctgaaggtaagtacccaaatcgcagcgcacatgatactggattagggcaatatgctaaggatgatgagtcattgactaaccacgatgacgttgtgtggatcacgacaggaaccactcatgtagcgcgtgcagaggagtggcctattatgccaacggaatgggcgcatgcacttctgaaaccttggaacttttttgatgagactccgacgttaggtgaaaagaagaaatga

SEQ ID NO: 128 - DNA, 909 bp, Klebsiella aerogenes
atggcgacagtagaatccggtcaagattatcagcgctggttggctaagattaaccaggtatgcgggcacttcgctgcacgcccgcttggagacgaatttcacggggaaattgatgcgtcgtacaccggttcgttgaaggtaagtacggtcacagcacgcggcgtaaacttatatcgtactcgtaacgagattaaacgtgacaacgacgcatggttctatacagtgttccagttggaagggcaagcagctattgagcaggatgagcgccaggtcgcgttagaggcaggagatattacattgatcgacgctagtcgcccctgcagtattttctggcaacacacatctaagcaggcatcacttcttcttccgcgccaattagtcgagagccaattgcatggaggagacatctctattgcgacacgtttgaataaaacgctgcctatggtccaattaagtcaacgtcttctgcaagaatctcttcgcagtcctgcattaagtgagtcggagggggaagcggcattagaggccattatctgtcttctgcgccccatgctgctgcaacaagacgctccgcctactcgccgtgataaacagttccaaaaaattatggctttgattgacgaatccatccaatctgagcaacttcgcccagaatggctggcgggcgaaacaggaatgagtgttcgtagtctgtaccgcctgttcgctgataaaggacttgtggttgctcagtatattaagaatcgccgcttggatctgtgtgctcgtgcattgcagaatgcacatgatgaagaaaaattggcagggatcgggtactcatggggcttctcagatcactcgcatttcagcacagcgttcaagcagcgcttcggcgtatccccaggcgagtatcgtaagcgttaccgctga

SEQ ID NO: 129 - DNA, 963 bp, Klebsiella pneumoniae
atgcccgaagccactcaaatgcaacaatgtggcaatatcgttatcactggggaaggtcgtatgatgacaactgcggagggtggtttagcatatcaacgctggttggcaaccattaaccaagtgtgtggtcactttgctgcgcgtcccttaaaggaaagtttccacggtgaaatcgatgctcgctatgcggggtcgttaaaggtgtctaccgttactgctgcaggagtcaacctttatcgcacgcgtaatgaaatcaaacgcgataacgatgcatggttctatactgtcttccagttggctggcgaagcgatcatcgaacaggatgatcgccaagtgacgctggcagcaggggatattacccttattgacgccgcccgcccatgtagcattgtatggcagcaaacatcgcgtcaagcgtcacttttattaccccgccaacgtgtagcaccgacaggtgatattacaacagcgtgtcgcttggacaagagccttcctatggtacaactgtcacaacgtcttttgttagagtcgatgggcggaacgactctgtccgcgtcagagtctgaggcggctctggaagcaatcgcctgcttattacgccccgtgcttcatcagcgtgatccagcgccgagtcgccgtgtcaaacagtttcagaagatcattgcgttaatcgatgctagtattcaaagcgaacacttgcgtcccgaatggttggcaagcgaaaccggaatgagtgtacgctctttataccgtctgtttgccgatcaagggctggtagttgcccagtacatcaagaatcgccgtcttgacttgtgtgcccaagctcttcagaatgtgcacgatgacgagaaattagccggtattggttatcgttggggattttctgaccattctcacttcagtactgctttcaagcagcgtttcggagttacgcccgggcagtaccgtaaacgctgtcgttaa

SEQ ID NO: 130 - DNA, 140 bp, Artificial Sequence
  Synthetic TynA construct
tatgaacgtctggcaaaactgacaaacaagctggcaaaagcaaacaaacccctttgcatcagtactgataatgtgaacctgactaaaccgcccacagagcgcggttgctaacaagaacacaacagaaagaggggttaata

(19) United States
(12) Patent Application Publication
(10) Pub. No.: US 2022/0372585 A1
(43) Pub. Date: Nov. 24, 2022

(54) Sensors for Aromatic Compounds and Methods of Making and Using Same

(71) Applicants: Tae Seok Moon, St. Louis, MO (US); Hyojeong Yi, St. Louis, MO (US); Matthew Amrofell, St. Louis, MO (US); Chenggang Xi, St. Louis, MO (US); Austin Rottinghaus, St. Louis, MO (US)

(72) Inventors: Tae Seok Moon, St. Louis, MO (US); Hyojeong Yi, St. Louis, MO (US); Matthew Amrofell, St. Louis, MO (US); Chenggang Xi, St. Louis, MO (US); Austin Rottinghaus, St. Louis, MO (US)

(73) Assignee: Washington University, St. Louis, MO (US)

(21) Appl. No.: 17/731,195

(22) Filed: Apr. 27, 2022

Related U.S. Application Data
(60) Provisional application No. 63/180,176, filed on Apr. 27, 2021.

(51) Int. Cl. C12Q 1/6897; C12Q 1/02; G01N 33/94

(52) U.S. Cl. CPC C12Q 1/6897; C12Q 1/02; G01N 33/9406; C12R 2001/19

(57) ABSTRACT

Among the various aspects of the present disclosure is the provision of molecular sensors, microbial sensors, constructs, systems, and methods for selectively detecting aromatic compounds.

### CROSS-REFERENCE TO RELATED APPLICATIONS

This application claims priority from U.S. Provisional Application Ser. No. 63/180,176 filed on Apr. 27, 2021, which is incorporated herein by reference in its entirety.

### STATEMENT REGARDING FEDERALLY SPONSORED RESEARCH OR DEVELOPMENT

This invention was made with government support under AT009741 awarded by the National Institutes of Health, CBET-1350498 and DGE-1745038 awarded by the National Science Foundation, N00014-17-1-2611 and N00014-19-1-2357 awarded by the Office of Naval Research, and 2020-33522-32319 awarded by the United States Department of Agriculture. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “019731-US-NP_Sequence Listing_ST25.txt” created on 27 Apr. 2022; 183,773 bytes). The subject matter of the Sequence Listing is incorporated herein by reference in its entirety.

### FIELD

The present disclosure generally relates to engineered probiotics and proteins for selective sensing.

### SUMMARY

Among the various aspects of the present disclosure is the provision of systems and methods for detecting aromatic compounds. An aspect of the present disclosure provides for a method of protein engineering (e.g., a regulator protein) comprising: mutagenizing specific amino acids in and around a ligand-binding site of a protein, wherein the mutagenizing enables changes in ligand-protein binding specificity while maintaining protein-DNA interaction and thus downstream gene expression control; and/or linking ligand-protein binding to output response. An aspect of the present disclosure provides for an engineered molecular sensor. In some embodiments, the sensor comprises an engineered regulator protein or enzyme (e.g., TrpR, TyrR, TynA, FeaR) comprising a ligand-protein binding site; a reporter (comprising a signaling moiety, e.g., GFP); and/or a promoter (e.g., PtyrP) capable of inducing or repressing the reporter in the presence or absence of a target aromatic compound bound to the engineered molecular sensor. In some embodiments, the engineered regulator protein or the ligand-protein binding site or a sequence encoding the engineered regulator protein or the ligand-protein binding site of the engineered regulator protein is at least about 80% identical to a WT regulator protein or a WT regulator ligand-protein binding site; or a sequence encoding the engineered regulator protein or ligand binding site thereof is at least about 80% identical to a sequence encoding the WT regulator protein or the WT regulator ligand-protein binding site; or functional fragment thereof, the engineered regulator protein having ligand binding activity. In some embodiments, the engineered molecular sensor is optionally genomically integrated into a microorganism, optionally selected from a probiotic or is a purified cell-free sensor. In some embodiments, the engineered regulator protein or enzyme is an engineered TrpR, TyrR, TynA, or FeaR protein. In some embodiments, the engineered regulator protein is an engineered TyrR and is an aromatic amino acid-specific sensor, wherein the aromatic amino acid-specific sensor is a phenylalanine (Phe)- or a tyrosine (Tyr)-specific sensor In some embodiments, the engineered regulator protein or enzyme is an engineered FeaR or TynA and is an aromatic amine-specific sensor, wherein the aromatic amine-specific sensor is a dopamine (DA)-, phenylethylamine (PEA)-, tyramine (Tyra)-, or tryptamine (Trypta)-specific sensor. In some embodiments, the engineered regulator protein is an engineered TrpR and is a tryptophan (Trp)-, 5-hydroxytryptophan (5-HTP)-, or tryptamine (Trpta)-specific sensor. In some embodiments, the engineered regulator protein or enzyme is an engineered TrpR, TyrR, TynA, or FeaR, and/or wherein the engineered TrpR, TyrR, TynA, or FeaR comprises at least one mutation to a ligand-protein binding site having specific amino acid binding activity. In some embodiments, PEA induces reporter expression; Phe induces reporter expression; or Tyr represses or induces reporter expression. In some embodiments, carboxylic acids 3,4-dihydroxyphenylacetic acid (DOPAC), phenylacetic acid (PAA), 4-hydroxyphenylacetic acid (HPPA), or indole-3-acetic acid (IAA) do not induce reporter expression or wherein IAA induces reporter expression. In some embodiments, the engineered regulator protein is an engineered TyrR and the engineered TyrR comprises one or more of the following mutations: E274Q+T14V mutations in TyrR inducing reporter expression in the presence of Phe; E274Q+D103S mutations in TyrR inducing reporter expression in the presence of Phe; or an R10F mutation in TyrR repressing reporter expression in the presence of Tyr. In some embodiments, TyrR protein sequence is at least about 80% identical to SEQ ID NO: 124 or TyrR ligand binding site is at least about 80% identical to residues 7-274 of SEQ ID NO: 124; a polypeptide encoded by SEQ ID NO: 11 or the WT ligand binding site sequence; or functional fragment or conservative substitution thereof of TyrR having ligand binding functional activity. Yet another aspect of the present disclosure provides for a TyrR-based selective or specific sensor specifically detecting phenylalanine (Phe) or tyrosine (Tyr) comprising: a TyrR or functional mutant or variant thereof; and/or a reporter gene (comprising a signaling moiety, e.g., GFP) operably linked to an inducible promoter (e.g., PtyrP promoter). In some embodiments, the inducible promoter comprises an activating response to Phe or a repressing response to Tyr or both Phe and Tyr. In some embodiments, TyrR is selected from: wild type (VVT) TyrR (SEQ ID NO: 124) or having at least about 80% identity to WT TyrR (SEQ ID NO: 124) and the reporter gene overexpresses if Phe is present; TyrR or TyrR having at least about 80% identity to TyrR comprising the mutation E274Q and the reporter gene overexpresses in the presence of Phe and Phe+Tyr; TyrR or TyrR having at least about 80% identity to TyrR comprising the mutation E274Q+T14V and the reporter gene overexpresses in the presence of Phe and Phe+Tyr and the reporter gene does not overexpress with Tyr in the absence of Phe; TyrR or TyrR having at least about 80% identity to TyrR comprising the mutation E274Q+D103S and the reporter gene overexpresses in the presence of Phe and Phe+Tyr and the reporter gene does not overexpress with Tyr in the absence of Phe; or TyrR or TyrR having at least about 80% identity to TyrR comprising the mutation R10F and the reporter gene is repressed with Tyr or Tyr+Phe and the reporter gene does not overexpress in the absence of Tyr. In some embodiments, the sensor is a target aromatic compound-inducible sensor or target aromatic compound-repressible sensor selected from: a Phe-inducible TyrR system (e.g., E274Q, E274Q+T14V, E274Q+D103H, E274Q+D103S), or Tyr-repressible TyrR system (e.g., R10F). In some embodiments, the TyrR-based sensor is for use to kinetically diagnose or treat disorders that cause Phe-dysregulation without interference from intestinal Tyr. In some embodiments, the TyrR-based sensor comprises E274Q+T14V or E274Q+D103S variants of TyrR. In some embodiments, the TyrR-based sensor is sensitive to Phe in the presence or absence of Tyr and does not respond to Tyr alone. In some embodiments, the TyrR-based sensor comprises R10F variant of TyrR and wherein the TyrR-based sensor exhibits about a 12-fold repression in the presence of Tyr independent of the presence of Phe. In some embodiments, the TyrR sensor has no significant response to Phe alone. In some embodiments, the TyrR-based sensor wherein a significant response is: under about 4000 au, under about 3000 au, or preferably at or under about 2000 au for Phe-inducible sensor; or above about 2000 au or preferably at or above 2000 au for a Phe-repressible sensor. In some embodiments, the engineered regulator protein or enzyme comprises an engineered FeaR and/or TynA and the engineered FeaR or TynA comprises one or more of the following mutations: a G494S mutation in TynA inducing reporter expression in the presence of PEA; a G494S mutation in TynA and A81T mutation in FeaR (e.g., G494S*) inducing reporter expression in the presence of PEA; a G494S mutation in TynA and A81S mutation in FeaR inducing reporter expression in the presence of PEA; a A81T or A81S mutation in FeaR inducing reporter expression in the presence of PEA and Tyra; a A81T mutation in FeaR inducing reporter expression in the presence of PEA; an A81T mutation in FeaR and S414M mutation in TynA inducing reporter expression in the presence of PEA; an A81T mutation in FeaR and G415H mutation in TynA inducing reporter expression in the presence of PEA; an A81 L, A81P, A81I, or A81N mutation in FeaR inducing reporter expression in the presence of PEA; a S414M or G415H mutation in TynA inducing reporter expression in the presence of Tyra; a G415H mutation in TynA inducing reporter expression in the presence of Tyra; a G415H mutation in TynA inducing reporter expression in the presence of Tyra and PEA; a M83Y mutation in FeaR inducing reporter expression in the presence of PEA; a M83N mutation in FeaR inducing reporter expression in the presence of Tyra; a Fear-KA mutant inducing reporter expression in the presence of Trypta; a tynA-KA or tynA-KP inducing reporter expression in the presence of Trypta; a I109N tynA-KA inducing reporter inducing expression in the presence of Trypta; a I109N FeaR-KA inducing reporter inducing expression in the presence of Trypta; a Q76, Q116, L108(T), W110(S), or W110(C) FeaR mutation; a D413 or Y496 TynA mutation; or PtynA-MG, TynA-KP and FeaR-KA inducing reporter inducing expression in the presence of Trypta and does not induce expression in the presence of dopamine (DA), phenylethylamine (PEA), or tyramine (Tyra). In some embodiments, the FeaR or TynA protein sequence or ligand binding site of FeaR or TynA is at least about 80% identical to a polypeptide encoded by the WT FeaR sequence of SEQ ID NO: 36 or WT TynA sequence of SEQ ID NO: 32 or WT ligand binding site or functional fragment or conservative substitution thereof and has ligand-binding activity. Yet another aspect of the present disclosure provides for a selective sensor for specifically detecting target aromatic compounds (e.g., Phe, Tyr, PEA, Tyra) comprising: a molecular sensor or an engineered microorganism (e.g., an E. coli strain) capable of expressing native or non-native FeaR and TynA (or functional fragments, conservative substitutions, mutants, or variants thereof); and/or a promoter-reporter system comprising a reporter gene under the control of a promoter capable of being induced or repressed by one or more target aromatic compounds (e.g., Phe, Tyr, PEA, Tyra) or enzymatic-reaction product thereof (e.g., aromatic aldehyde, aromatic carboxylic acid) and/or producing an output response (e.g., increased expression or repression). In some embodiments, the output response is a repression of the reporter (e.g., signaling or detection moiety, such as GFP) expression. In some embodiments, the output response is an overexpression of the reporter (e.g., signaling or detection moiety, such as GFP). In some embodiments, the selective sensor comprises one or more enzymes or transcription factors. Yet another aspect of the present disclosure provides for a selective sensor for specifically detecting aromatic amines (e.g., PEA, Tyra, DA, Trypta) or aromatic aldehydes thereof comprising: a TynA and FeaR protein (or a functional mutant or variant thereof); and/or a reporter gene (comprising a signaling moiety, e.g., GFP) operably linked to an inducible promoter (e.g., PtynA promoter). In some embodiments, the selective sensor induces expression of PtynA in response to aromatic aldehydes, but not aromatic amines. In some embodiments, the TynA protein or mutant variant thereof is selective to a specific aromatic amine. In some embodiments, TynA converts periplasmic amines (e.g., PEA, Tyra, DA, Trypta) to aldehydes which are imported into the cytoplasm; and in the cytoplasm, FeaR induces expression from the PtynA promoter expressing a reporter gene (e.g., detectable signal moiety) when in the presence of aldehydes. In some embodiments, the selective sensor is selective to PEA and Tyra. In some embodiments, either TynA or FeaR or both are engineered for selectivity. In some embodiments, the sensor comprises G494S mutation in TynA and/or optionally A81T mutation in the FeaR protein (i.e., double mutant sensor (G494S*)). In some embodiments, the selective sensor is a Tyra-specific variant, comprises G415H mutation in TynA. In some embodiments, G415H mutation in TynA induces expression of a reporter by about 200-fold and/or about 79-fold in response to Tyra and PEA, respectively, with minimal response to DA and Trypta. In some embodiments, A81T FeaR mutation with WT TynA is PEA and Tyra non-selective; S414M TynA and WT FeaR is slightly Tyra selective; S141M TynA and A81T FeaR is PEA selective; G415H TynA and WT FeaR is Tyra selective; or G415H TynA and A81T FeaR is PEA selective. In some embodiments, FeaR A81L, A81P, A81I, and A81N are PEA-specific variants. In some embodiments, Tyra-selective sensors (S414M or G415H TynA) can be transformed into PEA-specific sensors by introducing A81T FeaR; sensor sensitivity (G494S TynA) can be improved by A81T or A81S FeaR mutations; and/or PEA-Tyra selective sensors (A81T or A81S FeaR) can become PEA-specific sensors when combined with G494S TynA. In some embodiments, the engineered regulator protein is an engineered TrpR and the engineered TrpR comprises one or more of the following mutations: the TrpR variant is O_{D} or O₁, repressing reporter expression in the presence of Trp and 5-HTP, but not Trypta, wherein the protein sequence or ligand binding site sequence of TrpR is at least about 80% identical to a polypeptide encoded by the WT TrpR sequence of SEQ ID NO: 113 or WT ligand binding site sequence or functional fragment or conservative substitution thereof and/or has ligand-binding activity. In some embodiments, the reference nucleotide sequence encoding TrpR is SEQ ID NO: 113 and the reference amino acid sequence for TrpR is SEQ ID NO: 122; the reference nucleotide sequence encoding TyrR is SEQ ID NO: 11 and the reference amino acid sequence for TyrR is SEQ ID NO: 124; the reference nucleotide sequence encoding FeaR is SEQ ID NO: 36 and the reference amino acid sequence for FeaR is SEQ ID NO: 125; or the reference nucleotide sequence encoding TynA is SEQ ID NO: 32 and the reference amino acid sequence for TynA is SEQ ID NO: 123. In some embodiments, the engineered regulator protein or enzyme (e.g., TyrR, FeaR, TynA, or TrpR) or the ligand binding site of the engineered regulator protein or enzyme comprises at least 80% identity to a polypeptide encoded by SEQ ID NO: 32, SEQ ID NO: 36, SEQ ID NO: 11, or SEQ ID NO: 113; at least 80% identity to SEQ ID NO: 122 (TrpR WT), SEQ ID NO: 123 (tynA WT), SEQ ID NO: 124 (tyrR WT), or SEQ ID NO: 125 (fear WT); or at least 80% identity to D413 to Y496 of SEQ ID NO: 123, C7 to E274 of SEQ ID NO: 124, W12 to S118 of SEQ ID NO: 125, A81 to W110 of SEQ ID NO: 125, Q76 to Q116 of SEQ ID NO: 125, K72 to T83 of SEQ ID NO: 122, or a functional fragment or conservative substitution thereof. In some embodiments, the K_{A} value of the engineered regulator protein is less than the K_{A} value of the wild type regulator protein in the presence of a target aromatic compound; the K_{A} value of the engineered TyrR sensor is between about 0.05 mM and about 0.3 mM optionally in human intestines, serum, or urine; or the K_{A} value of the engineered TynA-FeaR is between about 0.001 mM and about 0.1 mM optionally in plasma or food. In some embodiments, the presence of a target aromatic compound induces or represses reporter gene expression of the engineered regulator protein or enzyme compared to the wild type regulator protein or enzyme. In some embodiments, the engineered regulator protein or enzyme has a selectivity, induction, or repression response that is greater than wild type. In some embodiments, the regulator protein or enzyme or the regulator protein or enzyme binding site is modified to increase selectivity. In some embodiments, the engineered molecular sensor is a ligand-specific biosensor for phenylalanine, tyrosine, phenylethylamine, or tyramine. In some embodiments, the engineered molecular sensor is directly transferred into probiotic organisms or purified for cell-free sensor application. Yet another embodiment provides for a selective sensor for specifically detecting target aromatic compounds (e.g., Phe, Tyr) comprising: an engineered microorganism (e.g., an E. coli strain) comprising the engineered molecular sensor capable of expressing native or non-native TrpR (or functional mutants or variants thereof); and/or a promoter-reporter system comprising a reporter gene (e.g., GFP) under the control of a promoter (e.g., Ptrp) capable of being induced or repressed by one or more target aromatic compounds and producing an output response (e.g., increased expression or repression of the reporter gene). In some embodiments, the TrpR is a TrpR variant O_{D} or O₁, each with a synthetic Ptrp promoter, the selective sensor having strong repression in the presence of Trp and 5-HTP, but not Trypta. In some embodiments, a genomic copy of wild-type trpR is knocked out from the engineered microorganism selected from an E. coli strain (e.g., EcN). In some embodiments, the WT TrpR system has strong repression of GFP expression with fold repressions of 120-fold, 20-fold, and 7-fold in response to Trp, 5-HTP, and/or Trpta, respectively. In some embodiments, the engineered TrpR variants maintain strong repression in the presence of Trp and 5-HTP, but not Trypta. In some embodiments, the O_{D} variant demonstrates about 5-fold and about 7-fold repression in response to Trp and/or 5-HTP, respectively. In some embodiments, the O₁ variant demonstrates about 60-fold and about 15-fold repression in response to Trp and 5-HTP, respectively. In some embodiments, the target aromatic compounds are one or more of tryptophan (Trp), 5-hydroxytryptophan (5-HTP), or tryptamine (Trypta). Yet another aspect of the present disclosure provides for an artificial DNA construct comprising, as operably associated components in the 5′ to 3′ direction of transcription: (a) a promoter functional in a microorganism (e.g., transgenic microorganism, wild type microorganism); (b) a first polynucleotide comprising a nucleotide sequence encoding (i) a first polypeptide having TrpR activity; (ii) a second polypeptide having TyrR activity; or (iii) a first polypeptide having FeaR activity and a second polynucleotide comprising a nucleotide sequence encoding a second polypeptide having TynA activity; (c) a reporter gene (e.g., GFP); and/or (d) a transcriptional termination sequence. In some embodiments, the microorganism is capable of expressing native or non-native (i) TrpR; (ii) TyrR; or (iii) FeaR and TynA (or functional mutants or variants thereof). In some embodiments, the microorganism specifically expresses or represses reporter gene expression compared to a microorganism not comprising the artificial DNA construct in the presence or absence of aromatic compounds. Yet another aspect of the present disclosure provides for a microbial sensor selected from an engineered wild type or transgenic microorganism transformed with the artificial DNA construct. In some embodiments, the wild type or transgenic microorganism is selected from Escherichia coli Nissle 1917 (EcN), DH10B, or E. coli MG1655. In some embodiments, the selective sensor is selective for an aromatic compound and/or the aromatic compound is an aromatic amino acid selected from phenylalanine (Phe), tyrosine (Tyr), and/or tryptophan (Trp). In some embodiments, the selective sensor is selective for an aromatic compound and the aromatic compound is an aromatic amine neurochemical selected from dopamine (DA), phenylethylamine (PEA), tyramine (Tyra), tryptamine (Trypta), serotonin, epinephrine, or norepinephrine. In some embodiments, a TrpR-based sensor is selective for Trp; a TyrR-based sensor is selective for Phe and/or Tyr; and/or a TynA-FeaR sensor system is selective for aromatic amines. Yet another aspect of the present disclosure provides for a ligand-specific sense-and-respond system, comprising purified sensors or engineered proteins or probiotics for specific sensing of aromatic compounds (e.g., amino acids, aromatic amines, aromatic neurochemicals) comprising: providing a orthogonal DNA-TF binding system with accompanying selectivity changes; changing ligand-TF binding specificity by leveraging differential multimerization patterns of TyrR without affecting DNA-TF binding interaction; or a “dual-control knob” strategy to improve the specificity and sensitivity of substrate-enzyme and ligand-TF interaction while maintaining DNA-TF binding interaction. In some embodiments, target ligands are structurally similar and ligand-protein binding controls downstream functions such as reporter gene expression. In some embodiments, the ligand-specific sense-and-respond system comprises an engineered microorganism. Yet another aspect of the present disclosure provides for a method of using the engineered molecular sensor, comprising obtaining or having obtained a biological sample from a subject and/or contacting the biological sample with the engineered molecular sensor. In some embodiments, the subject has an aromatic compound-associated disease, disorder, or condition. In some embodiments, elevated levels of Phe detected by the sensor indicate the subject has phenylketonuria. In some embodiments, elevated levels of Tyr detected by the sensor indicate the subject has type 2 tyrosinemia. In some embodiments, elevated levels of PEA detected by the sensor indicate the subject has a psychological disorder. In some embodiments, the presence of Tyra detected by the sensor indicates catecholamine release and/or an increase in blood pressure. In some embodiments, the presence of Trypta detected by the sensor causes serotonin release and/or stimulation of gastrointestinal motility. Yet another aspect of the present disclosure provides for a method of using the engineered molecular sensor, comprising monitoring food quality or diagnosing or treating metabolic, digestive, and/or neurological disorders. In some embodiments, the presence of PEA, Tyra, and/or Trypta in food detected by the sensor indicates microbial contamination. In some embodiments, the sensor dynamically identifies microbial contamination in consumable products, manages various debilitating neurological disorders, or normalizes dysregulated metabolites associated with metabolic disorders. In some embodiments, the sensor recognizes or is selective for aromatic metabolites associated with various metabolic or neurological disorders or medical conditions. In some embodiments, the aromatic compounds are selected from phenylalanine (Phe) or tyrosine (Tyr). In some embodiments, the aromatic compounds are neurochemicals. In some embodiments, the neurochemicals are selected from aromatic neurotransmitters or neuromodulators. In some embodiments, the neurochemicals are selected from dopamine (DA), phenylethylamine (PEA), tyramine (Tyra), tryptamine (Trypta), serotonin, epinephrine, or norepinephrine. In some embodiments, the subject has or is suspected of having a medical condition associated with elevation, presence, or absence of aromatic compounds. In some embodiments, the sensor differentiates metabolites with divergent functions even having structural similarity. In some embodiments, the sensor modulates the specificity of ligand-protein binding while maintaining protein-DNA interactions and thus downstream gene expression control. Yet another aspect of the present disclosure provides for a method of using the engineered molecular sensor, the method comprising monitoring food quality, diagnosing or treating metabolic, digestive, and/or neurological disorders in probiotics or ex vivo wearable, paper-based or cell-free systems, or dynamically regulating enzymatic pathways for microbial metabolic engineering using the engineered molecular sensor. Yet another aspect of the present disclosure provides for a method of protein engineering (e.g., a regulator protein or enzyme) comprising: mutagenizing specific amino acids in and around a ligand-binding site of a protein or enzyme (e.g., TrpR, TyrR, FeaR, TynA), wherein the mutagenizing enables changes in ligand-protein binding specificity while maintaining protein-DNA interaction and thus downstream gene expression control; and/or linking ligand-protein binding to output response (e.g., promoter-reporter gene system).

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1C. Phenylalanine- and tyrosine-dependent regulation of the PtyrP promoter by TyrR is tyrosine-dominant (A) Structures of the two TyrR ligands phenylalanine (Phe) and tyrosine (Tyr). Structural differences between ligands are shown in red. (B) Schematic of how TyrR regulates the PtyrP promoter in a Phe- and Tyr-dependent manner. (C) Transfer curves for PtyrP with plasmid-based overexpression of wild-type TyrR in response to Phe, Tyr, and the specified concentrations of both Phe and Tyr. Points represent experimental data while lines represent the fitted curves (see e.g., TABLE 2). Values and error bars are the average and standard deviation of biological triplicate, respectively. See also FIG. 8A-FIG. 8C.

FIG. 2A-FIG. 2G. Development of phenylalanine- and tyrosine-specific sensors (A) Response of the PtyrP promoter to 1 mM phenylalanine (Phe), 1 mM tyrosine (Tyr), and both 1-mM Phe and 1-mM Tyr with plasmid-based overexpression of wild type (WT), E274Q mutant, and R10F mutant TyrR. (B) Structure of the Phe-binding pocket in the N-terminal regions of the TyrR homodimer. TyrR monomers are in white and magenta. Selected amino acids with known roles in Phe-dependent modulation of RNA polymerase are shown in elemental coloring: carbon atoms are shown in green, nitrogen in blue, oxygen in red, and sulfur in yellow. The structure was obtained from RCSB protein data bank (Reference ID 2JHE), and the image was generated using PyMOL. (C) Modulation of the Phe-induction threshold for the PtyrP promoter by saturation mutagenesis of the TyrR's Phe-binding pocket. Two TyrR mutants, E274Q+T14V and E274Q+D103S, were obtained with increased K_{A} values (2-fold and 3-fold higher than the E274Q mutant, respectively). (D-G) Transfer curves for PtyrP with plasmid-based overexpression of E274Q (D), E274Q+T14V (E), E274Q+D103S (F), and R10F (G) TyrR variants in response to Phe, Tyr, and the specified concentrations of both Phe and Tyr. Points represent experimental data while lines represent the fitted curves (see e.g., TABLE 2). The responses to Tyr (D-F) or Phe (G) were not fit due to a lack of response. Values and error bars are the average and standard deviation of biological triplicate, respectively. AA, amino acid. See also FIG. 9A-FIG. 9B, FIG. 10A-FIG. 10D, and FIG. 11-FIG. 12.

FIG. 3A-FIG. 3D. Development and characterization of sensors for aromatic amines. (A) Structures of the four TynA substrates phenylethylamine (PEA), tyramine (Tyra), dopamine (DA), and tryptamine (Trypta) as well as the corresponding aldehyde ligands for FeaR. Structural differences between ligands are shown in green. (B) Schematic of aromatic amine sensing using the TynA-FeaR system. The TynA enzyme converts periplasmic amines to aldehydes, which are imported into the cytoplasm. In the presence of the aldehydes, FeaR induces expression from the PtynA promoter. (C) Validation that FeaR regulates PtynA in response to aldehydes and not amines. DA induces PtynA expression in the presence, but not absence of TynA. (D) Transfer curves of PtynA after 24 h of induction with DA, PEA, Tyra, and Trypta. Points represent experimental data while lines represent the fitted curves (see e.g., TABLE 2). Values and error bars are the average and standard deviation of biological triplicate, respectively. See also FIG. 13 and FIG. 14.

FIG. 4A-FIG. 4D. Identification of phenylethylamine and tyramine-selective sensors through TynA engineering. (A) Structure of the TynA catalytic domain. Unmutagenized amino acids with known catalytic roles are in red. Amino acids selected for mutagenesis are in blue. The structure was obtained from RCSB protein data bank (Reference ID 6EZZ), and the image was generated using PyMOL. (B) GFP fluorescence from PtynA when induced for 24 h with 0 mM and 1 mM dopamine (DA), phenylethylamine (PEA), tyramine (Tyra), and tryptamine (Trypta). PtynA was regulated by wild-type TynA (left), G494S TynA (middle), or G415H TynA (right) and either wild-type or A81T (*) FeaR. (C and D) Transfer curves of PtynA after 24 h of induction with DA, PEA, Tyra, and Trypta. PtynA was regulated by the G494S TynA mutant with the A81T FeaR mutant (C) or the G415H TynA mutant with wild-type FeaR (D). Points represent experimental data while lines represent the fitted curves (see e.g., TABLE 2). The responses of G494S* to DA, Tyr, and Trypta and the responses of G415H to DA and Trypta were not fit due to a lack of response. Values and error bars are the average and standard deviation of biological triplicate, respectively. See also FIG. 15-FIG. 19.

FIG. 5A-FIG. 5D. Engineering FeaR to identify improved phenylethylamine-specific sensors (A) Computationally predicted ligand-binding region of FeaR. Key residues are shown with (magenta) and without (green) phenylethylamine-aldehyde (PEA-aldehyde; cyan) complexed. The structure was predicted using the Robetta web server. Ligand docking was modeled using the Rosetta Ligand Docking Server hosted by ROSIE. The image was generated using PyMOL. (B) Fluorescence from PtynA when induced for 24 h with 0.25 mM dopamine (DA), phenylethylamine (PEA), tyramine (Tyra), and tryptamine (Trypta). PtynA was regulated by wild-type TynA and 20 different FeaR variants with the specified amino acid in the 81st position. Residues for each group are listed from left to right in order of increasing size. Non-zero values and error bars are the average and standard deviation of biological triplicate, respectively. Variants with significant expression in response to PEA were determined by unpaired t tests (p<0.05). (C) Transfer curve of PtynA after 24 h of induction with DA, PEA, Tyra, and Trypta. PtynA was regulated by wild-type TynA and the A81L FeaR mutant. Points represent experimental data while the line represents the fitted curve (see e.g., TABLE 2). The responses to DA, Tyr, and Trypta were not fit due to a lack of response. Values and error bars are the average and standard deviation of biological triplicate, respectively. (D) Reorganization of FIG. 6B with respect to the size and hydropathy index of the 81st residue (see e.g., TABLE 3). The blue area represents characteristic values that permit binding of Tyra. See also FIG. 20-FIG. 25.

FIG. 6A-FIG. 6E. Engineering additional FeaR amino acids to identify amine-specific sensors. (A-C) Fluorescence from PtynA when induced for 24 h with 0.25 mM dopamine (DA), phenylethylamine (PEA), tyramine (Tyra), and tryptamine (Trypta). PtynA was regulated by wild-type TynA and 20 different FeaR variants with the specified amino acid in the (A) 83rd, (B) 108th, or (C) 110th position. Residues for each group are listed with their size and hydropathy index (see e.g., TABLE 3). Values are the average of biological triplicate. To obtain the relative fluorescence, fluorescence values for each variant were normalized to the fluorescence of the wild-type sensor (see Methods; Equation 3). (D and E) Transfer curves of PtynA after 24 h of induction with DA, PEA, Tyra, and Trypta. The expression of PtynA was regulated by wild-type TynA with (D) M83Y or (E) M83N FeaR variants. Points represent experimental data while lines represent the fitted curves (see Methods and TABLE 2). Values and error bars are the average and standard deviation of biological triplicate, respectively. See also FIG. 26-FIG. 29.

FIG. 7 is a schematic depicting creation of specific sensors for structurally similar amino acids and neurochemicals.

FIG. 8A-FIG. 8C. Promoter screen for the TyrR regulon, related to FIG. 1A-FIG. 1C. (A) Schematic for how TyrR regulates promoters in a phenylalanine (Phe)- and tyrosine (Tyr)-dependent manner. In the absence of its amino acid (AA) ligands or in the presence of Phe, TyrR exists as homodimers that bind to high-affinity binding sites (strong boxes). When bound to Tyr, TyrR forms hexamers that bind to both the high-affinity and low-affinity (weak boxes) binding sites. TyrR alone is unable to interact with RNA polymerase (RNAP). However, AA-TyrR complexes induce expression of the promoter when bound to DNA upstream of the −35 site (as for Pmtr and the strong box for PtyrP). In contrast, AA-TyrR complexes repress expression from the promoter when bound to DNA overlapping or downstream of the −35 site (as for PtyrR, PtyrB, ParoF, ParoP, and the weak box for PtyrP). (B) TyrR-regulated promoter screen with wild-type expression of TyrR. Two known TyrR-inducible promoters showed significant induction in response to Phe or Tyr, while known repressible promoters showed only weak repression (at best 3-fold) in response to Phe or Tyr. The wild-type PtyrB promoter showed no detectable expression. Statistical comparisons were performed using two-tailed unpaired t-tests (ns, P>0.05; *, P<0.05; ***, P<0.001; ****, P<0.0001). (C) When TyrR was overexpressed, TyrR-mediated repressions of ParoF and ParoP were improved. Both the ParoF and ParoP promoters were mutated (*) to increase the expression level (see e.g., TABLE 6 for sequences). Values and error bars are the average and standard deviation of biological triplicate, respectively. AA, amino acid.

FIG. 9A-FIG. 9B. Potential TyrR variants with reduced sensitivity to phenylalanine, related to FIG. 2A-FIG. 2G. (A and B) Six TyrR E274Q variants were identified from the eight amino acid saturation mutagenesis libraries as having a reduced sensitivity to phenylalanine (Phe). Fluorescence values (A) were normalized by their maximum induction value (B). Each mutant showed a reduced level of induction relative to E274Q TyrR in response to 0.01 mM and 0.05 mM Phe. All values are from a single experiment.

FIG. 10A-FIG. 10D. Phenylalanine response of non-selected TyrR mutants, related to FIG. 2A-FIG. 2G. (A-D) PtyrP transfer curves with respect to phenylalanine (Phe) for TyrR variants E274Q+C7V (A), E274Q+L11G (B), E274Q+T14G (C), and E274Q+D103H (D). Points represent experimental data while lines represent the fitted curves (see Methods and TABLE 2). Values and error bars are the average and standard deviation of biological triplicate, respectively.

FIG. 11. Screening potential tyrosine-selective TyrR variants, related to FIG. 2A-FIG. 2G. Five potential tyrosine (Tyr)-selective TyrR variants were selected from the eight saturation mutagenesis libraries. Each variant was screened for its response to 0 mM amino acids (AA), 1 mM phenylalanine (Phe), 1 mM Tyr, and both 1 mM Phe and 1 mM Tyr. The R10F TyrR mutant was selected as the best Tyr-selective sensor. Values and error bars are the average and standard deviation of biological triplicate, respectively. AA, amino acid.

FIG. 12. Phenylalanine and tyrosine sensors are fully induced or repressed in rich medium, related to FIG. 2A-FIG. 2G. Response of the PtyrP promoter to 0 mM and 1 mM phenylalanine (Phe), 1 mM tyrosine (Tyr), and both 1 mM Phe and 1 mM Tyr in LB medium. The expression of PtyrP was regulated by wild-type (VVT) TyrR or four TyrR mutant variants. Values and error bars are the average and standard deviation of biological triplicate, respectively. AA, amino acid.

FIG. 13. Comparison of genomic context of feaR, feaB, and tynA in five selected E. coli strains, related to FIG. 3A-FIG. 3D. Genomic feaR, feaB, and tynA genes and their adjacent genes are shown for five selected E. coli strains: E. coli Nissle 1917 (EcN), uropathogenic E. coli UT1891 (UPEC), E. coli K12 MG1655, E. coli DH5a, and enterohaemorrhagic E. coli EDL933 (EHEC). Genes are shown as arrows with same colors represent homologs.

FIG. 14. PtynA transfer curves after 5 h of induction, related to FIG. 3A-FIG. 3D. Transfer curves of PtynA after 5 h of induction with dopamine (DA), phenylethylamine (PEA), tyramine (Tyra), and tryptamine (Trypta). Points represent experimental data while lines represent the fitted curves (see Methods and TABLE 2). Values and error bars are the average and standard deviation of biological triplicate, respectively.

FIG. 15. Full protein structure for TynA, related to FIG. 4A-FIG. 4D. Structure of a TynA homodimer. Monomers are in white and magenta. Amino acids with known catalytic roles are in red. Amino acids selected for random mutagenesis are in blue. The structure was obtained from RCSB Protein Data Bank (Reference ID 6EZZ), and the image was generated using PyMOL.

FIG. 16. Growth-based dual selection method for identifying ligand-selective aromatic amine sensors, related to FIG. 4A-FIG. 4D. TynA was mutagenized to alter the specificity of the enzyme to its amine substrates. Wild-type FeaR non-selectively binds to the aldehyde products and induces expression from the PtynA promoter. PtynA drives expression of a GFP reporter, the beta-lactamase Bla, and the levansucrase SacB. In the presence of carbenicillin, PtynA induction and Bla expression allows for cell survival (positive selection). In the presence of sucrose, PtynA induction and SacB expression causes cell death (negative selection). Ligand-selective TynA variants should induce expression of Bla in the presence of the ligand, but not induce expression of SacB in the presence of alternative ligands, allowing the cell to survive in the presence of carbenicillin or sucrose.

FIG. 17A-FIG. 17B. Transfer curves for the optimal phenylethylamine-specific or tyramine-selective sensors, related to FIG. 4A-FIG. 4D. (A and B) Transfer curves of PtynA after 5 h of induction with dopamine (DA), phenylethylamine (PEA), tyramine (Tyra), and tryptamine (Trypta). The expression of PtynA was regulated by the G494S TynA mutant with the A81T FeaR mutant (A) or the G415H TynA mutant with wild-type FeaR (B). Points represent experimental data while lines represent the fitted curves (see Methods and TABLE 2). The responses of G494S* to DA, Tyra, and Trypta and the responses of G415H to DA and Trypta were not fit due to a lack of response. Values and error bars are the average and standard deviation of biological triplicate, respectively.

FIG. 18A-FIG. 18B. Transfer curves for the phenylethylamine-specific G494S sensor, related to FIG. 4A-FIG. 4D. (A and B) Transfer curves of PtynA after 5 h (A) and 24 h (B) of induction with dopamine (DA), phenylethylamine (PEA), tyramine (Tyra), and tryptamine (Trypta). The expression of PtynA was regulated by the G494S TynA mutant with wildtype FeaR. Points represent experimental data while lines represent the fitted curves (see Methods and TABLE 2). The responses to DA, Tyra, and Trypta were not fit due to a lack of response. Values and error bars are the average and standard deviation of biological triplicate, respectively.

FIG. 19A-FIG. 19B. Validation of tyramine-selective TynA mutants, related to FIG. 4A-FIG. 4D. (A and B) GFP fluorescence from PtynA when induced with 0 mM and 1 mM dopamine (DA), phenylethylamine (PEA), tyramine (Tyra), and tryptamine (Trypta) for 5 h (A) and 24 h (B). PtynA was regulated by wild-type (WT), S414M, or G415H TynA and wild-type FeaR. G415H was identified as the most Tyra-selective sensor based on t-test. Values and error bars are the average and standard deviation of biological triplicate, respectively. Statistical comparisons were performed using two-tailed unpaired t-tests (ns, P>0.05; *, P<0.05; **, P<0.01).

FIG. 20. Location of A81 within FeaR, related to FIG. 5A-FIG. 5D. Computationally predicted structure of FeaR. Residue A81 (arrow) is oriented towards the interior of a solvent-accessible beta-barrel. The magenta region is the predicted ligand-binding domain and the cyan region is the predicted DNA-binding domain. The structure was predicted using the Robetta Web Server, and the image was generated using PyMOL.

FIG. 21. Effect of the A81T FeaR mutation on ligand selectivity, related to FIG. 5A-FIG. 5D. Reporter fluorescence from PtynA when induced for 24 h with 0 mM and 1 mM dopamine (DA), phenylethylamine (PEA), tyramine (Tyra), and tryptamine (Trypta). PtynA was regulated by wild-type (VVT), S414M, or G415H TynA and WT or A81T FeaR. Values and error bars are the average and standard deviation of biological triplicate, respectively. Statistical comparisons were performed using two-tailed unpaired t-tests (ns, P>0.05; *, P<0.05; **, P<0.01).

FIG. 22A-FIG. 22B. The A81T FeaR mutation improves sensitivity to phenylethylamine aldehyde and thus increases selectivity, related to FIG. 5A-FIG. 5D. (A and B) Transfer curves of PtynA after 24 h of induction with phenylethylamine (PEA). The expression of PtynA was regulated by wild-type TynA (A) or G494S TynA (B) and FeaR with and without the A81T or A81S mutations. Points represent experimental data while lines represent the fitted curves (see Methods and TABLE 2). Values and error bars are the average and standard deviation of biological triplicate, respectively.

FIG. 23A-FIG. 23B. Transfer curves for the phenylethylamine-specific G494S TynA and A81S FeaR sensor, related to FIG. 5A-FIG. 5D. (A and B) Transfer curves of PtynA after 5 h (A) and 24 h (B) of induction with dopamine (DA), phenylethylamine (PEA), tyramine (Tyra), and tryptamine (Trypta). The expression of PtynA was regulated by G494S TynA and A81S FeaR. Points represent experimental data while lines represent the fitted curves (see Methods and TABLE 2). The responses to DA, Tyra, and Trypta were not fit due to a lack of response. Values and error bars are the average and standard deviation of biological triplicate, respectively.

FIG. 24. PtynA transfer curves after 5 h of induction with the phenylethylamine-specific A81 L FeaR sensor, related to FIG. 5A-FIG. 5D. Transfer curves of PtynA after 5 h of induction with dopamine (DA), phenylethylamine (PEA), tyramine (Tyra), and tryptamine (Trypta). The expression of PtynA was regulated by wild-type TynA with the A81L FeaR mutant. Points represent experimental data while the line represents the fitted curve (see Methods and TABLE 2). The responses to DA, Tyra, and Trypta were not fit due to a lack of response. Values and error bars are the average and standard deviation of biological triplicate, respectively.

FIG. 25A-FIG. 25B. Transfer curves for the phenylethylamine-specific A81P FeaR sensor, related to FIG. 5A-FIG. 5D. (A and B) Transfer curves of PtynA after 5 h (A) and 24 h (B) of induction with dopamine (DA), phenylethylamine (PEA), tyramine (Tyra), and tryptamine (Trypta). The expression of PtynA was regulated by wild-type TynA with the A81P FeaR mutant. Points represent experimental data while lines represent the fitted curves (see Methods and TABLE 2). The responses to DA, Tyra, and Trypta were not fit due to a lack of response. Values and error bars are the average and standard deviation of biological triplicate, respectively.

FIG. 26. Ligand-binding pocket of different FeaR variants, related to FIG. 5A-FIG. 5D and FIG. 6A-FIG. 6E. Ligand-binding pocket of the computationally predicted structures of promiscuous wild-type A81 (green), PEA/Tyra-selective T81 (magenta), PEA-specific L81 (cyan), and nonfunctional H81 (orange) FeaR. Structures were predicted using the Robetta Web Server and the image was generated using PyMOL.

FIG. 27A-FIG. 27C. Engineering additional FeaR amino acids to identify amine-specific sensors, related to FIG. 6A-FIG. 6E. (A-C) Fluorescence from PtynA when induced for 24 h with 0.25 mM dopamine (DA), phenylethylamine (PEA), tyramine (Tyra), and tryptamine (Trypta). PtynA was regulated by wild-type TynA and 20 different FeaR variants with the specified amino acid in the (A) 83rd, (B) 108th, or (C) 110th position. Residues for each group are listed from left to right in order of increasing size. Values and error bars are the average and standard deviation of biological triplicate, respectively.

FIG. 28A-FIG. 28D. Transfer curves for Tyra-specific FeaR variants, related to FIG. 6A-FIG. 6E. Transfer curves of PtynA after 24 h of induction with dopamine (DA), phenylethylamine (PEA), tyramine (Tyra), and tryptamine (Trypta). The expression of PtynA was regulated by wild-type TynA with (A) M83Q, (B) L108T, (C) W110S, or (D) W110C FeaR variants. Points represent experimental data while lines represent the fitted curves (see Methods and TABLE 2). The responses to DA, Tyra, and Trypta were not fit due to a lack of response. Values and error bars are the average and standard deviation of biological triplicate, respectively.

FIG. 29A-FIG. 29B. TynA-FeaR sensor activity and specificity in richer media, related to FIG. 6A-FIG. 6E. (A and B) Fluorescence from PtynA when induced for 24 h with 0 mM and 1 mM dopamine (DA), phenylethylamine (PEA), tyramine (Tyra), tryptamine (Trypta), 3,4-dihydroxyphenylacetic acid (DOPAC), phenylacetic acid (PAA), 4-hydroxyphenylacetic acid (HPAA), and indole-3-acetic acid (IAA) in (A) M9+2% glycerol+0.2% casamino acids or (B) LB. PtynA was regulated by the specified variants of TynA and FeaR. The response of the G494S TynA mutant sensors was not determined (ND) for DOPAC, PAA, HPAA, or IAA. Values and error bars are the average and standard deviation of biological triplicate, respectively.

FIG. 30. Schematic design of biosensor development for tryptamine specific biosensors by genetic parts swapping. Three types of tynA and feaR genes from E. coli K-12 MG1655 (MG), Klebsiella aerogenes (KA) and Klebsiella pneumoniae (KP), and two types of PtynA sequences were chosen and tested in 18 different combinations. PtynA-KP/KA was constructed by replacing the FeaR binding sites of PtynA-MG with those from Klebsiella species. Sequences of those constructs are shown in TABLE 7.

FIG. 31. Significant enhancement of fluorescence intensity induced by Tryptamine in sensor systems containing FeaR-KA. GFP fluorescence from PtynA when induced for 24 h with 0 mM (None) and 0.5 mM dopamine (DA), phenylethylamine (PEA), tyramine (Tyra), and tryptamine (Trypta). PtynA from MG or KP/KA was regulated by TynA from MG or KP and FeaR from MG or KA. Data with TynA from KA and FeaR from KP were not included since they showed very low activity. Values and error bars are the average and standard deviation of biological triplicate, respectively.

FIG. 32. Tryptamine specific variant I109N. GFP fluorescence from PtynA-MG when induced for 24 h with 0 mM (None) and 0.5 mM dopamine (DA), phenylethylamine (PEA), tyramine (Tyra), and tryptamine (Trypta). Sensor system with PtynA-MG, TynA-KP and FeaR-KA was chosen as the wild type (WT) for further directed evolution. TynA-KP was employed to reduce the possible interference from DA during evolution processes. Values and error bars are the average and standard deviation of biological triplicate, respectively.

FIG. 33A-FIG. 33B. Transfer curves for the tryptamine-specific FeaR-KA I109N sensor, related to FIG. 32. Transfer curves of PtynA after 5 h (A) and 24 h (B) of induction with dopamine (DA), phenylethylamine (PEA), tyramine (Tyra), and tryptamine (Trypta). The expression of PtynA-MG was regulated by the TynA-KP with FeaR-KA I109N mutant. Points represent experimental data while lines represent the fitted curves. The responses to DA, Tyra, and Trypta were not fit due to a lack of response. Values and error bars are the average and standard deviation of biological triplicate, respectively.

### DETAILED DESCRIPTION

The present disclosure is based, at least in part, on the development of the first ligand-specific or selective sensors for aromatic compounds such as aromatic neurochemicals or aromatic amino acids.

Examples of aromatic amino acids can be phenylalanine (Phe) and tyrosine (Tyr). Examples of aromatic neurochemicals (e.g., neurotransmitters, neuromodulators) can be aromatic amines, such as phenylethylamine (PEA) and tyramine (Tyra), which are structurally similar and have been implicated in a variety of medical conditions.

As described herein, biosensors with high selectivity for aromatic compounds, such as phenylalanine, tyrosine, phenylethylamine, or tyramine, were developed and demonstrated for the first time.

Applications for this technology can include:

1) developing medically-relevant probiotic sensors with high specificity, which is critical to differentiate metabolites with divergent functions and create smart probiotics for accurate diagnostic tools; and

2) developing autonomous microbes that dynamically identify microbial contamination in consumable products, manage various debilitating neurological disorders, and normalize dysregulated metabolites associated with metabolic disorders.

Publically available or computational tools purchased include: the MOTIF Search webtool (genome.jp), Robetta Web Server, Chem3D 16.0 (PerkinElmer, Waltham, Mass.), Rosetta Ligand Docking Server hosted by ROSIE, KEGG database, https://www.hmpdacc.org/HMRGD/, Interactive tree of life (iTOL) phylogeny tree annotation tool, HMP database, Greengenes, NCBI, and PyMOL 2.4.1 (Schrödinger, Inc., New York, N.Y.).

Engineered Microorganism

Many probiotics have been engineered for therapeutic applications, including treating various cancers, inflammatory disorders, metabolic disorders, and bacterial infections. Many of these probiotics constitutively express the proteins, raising concerns of off-target effects and genetic stability. Several engineered probiotics partially mitigate this concern by using sensors that limit protein expression to targeted biogeographical regions. For example, probiotics have been designed to regulate the therapeutic expression through sensors that detect oxygen content. However, they should also be engineered with biosensors that recognize the disease state by measuring the concentrations of relevant metabolites. By using disease-specific sensors, therapeutics can be delivered with both geographical and temporal precision. In addition, these sensors expand the potential applications of the engineered probiotics, which can be used to diagnose numerous disorders.

To develop these smart probiotics, the toolbox of sensors needs to be expanded for common probiotic microbes. Synthetic sensors can be created using several protein design approaches. However, sensors can also be obtained more efficiently by mining sensors that naturally exist in microbes. These sensors can often be directly transferred into probiotic organisms for use in various applications. However, most natural sensors recognize multiple structurally similar, but functionally diverse, metabolites often found in close proximity. To accurately correlate the concentration of a chemical with biological outcomes, engineered microbes need to differentiate between different chemicals. In addition, sensors often have an inadequate sensitivity to the ligand or lack orthogonality with native regulatory pathways in the probiotic, limiting their utility.

Specific sensing is critical for many probiotic sensor applications. Chronically elevated levels of structurally similar phenylalanine (Phe) and tyrosine (Tyr) are associated with the distinct disorders phenylketonuria and type 2 tyrosinemia, respectively. Sensors for both metabolites have been generated in different E. coli strains. However, the sensors are based on the wild-type version of the multi-ligand responsive TyrR transcription factor from E. coli and have limited selectivity and low dynamic ranges, making them unsuitable for differentiating between the two disorders. Similarly, the structurally similar amines phenylethylamine (PEA), tyramine (Tyra), and tryptamine (Trypta) are all commonly found and produced in the gut, but contribute to distinct biological outcomes. For example, extreme levels of PEA have been associated with a variety of psychological disorders, the presence of Tyra leads to catecholamine release and an increase in blood pressure, and the presence of Trypta causes serotonin release and the stimulation of gastrointestinal motility. Additionally, the presence of PEA, Tyra, and Trypta in food are indicators of microbial contamination, and eating foods with high levels of Tyra can lead to poisoning. Currently, there are no biosensors with high ligand specificity for these medically relevant chemicals, which can be employed in probiotic microbes.

A variety of protein engineering methods have been demonstrated for optimizing the selectivity and sensitivity of protein and RNA sensors. These approaches include directed evolution, rational design, and computational de novo design. Although each method has advantages, rational engineering can uniquely be performed using both basic knowledge of the protein structure and small library sizes, allowing for rapid construction and screening of libraries. In addition, when the structure of the protein is unknown, conserved or essential residues can also be identified through computational simulations or by aligning the sequence of the sensor with other proteins in the same family. Despite advances in protein engineering, creating ligand-specific sense-and-respond systems remains challenging due to the need to couple subtle protein conformational changes with differential protein-DNA interactions and gene expression control, especially when the target ligands are structurally similar.

Here is described the generation of sensors for a variety of disease-relevant aromatic metabolites in probiotic Escherichia coli Nissle 1917 (EcN). These metabolites include aromatic amino acids Phe, Tyr, and tryptophan (Trp), which are required to synthesize proteins and diverse essential metabolites, and aromatic amine neurochemicals dopamine (DA), PEA, Tyra, and Trypta, which are associated with many health issues. To generate sensors for these aromatic metabolites, three sensor systems were identified and engineered: the TrpR (Trp) sensor, the TyrR (Phe and Tyr) sensor, and the TynA-FeaR (aromatic amine) sensor system. Multiple engineered TrpR sensors were first characterized, which were previously created to be orthogonal to the wild-type E. coli-native system, and how the mutations impact the selectivity of the sensors was assessed. Next, the ligand selectivity of the TyrR and TynA-FeaR sensor systems was engineered and their sensitivity was tuned by rationally selecting and individually mutating amino acids in TyrR, TynA, and FeaR. This method of protein engineering quickly generates multiple small libraries that can be efficiently screened. Altogether, the first sensors selective for Phe, Tyr, PEA, or Tyra were generated. In engineering FeaR, novel insights into the uncharacterized structure of FeaR are also provided and residues important for ligand binding were identified for the first time. This work provides sensors with diverse clinical applications in engineering smart probiotics as well as a generalizable approach to modulating the specificity of ligand-protein binding while maintaining protein-DNA interactions and thus downstream gene expression control.

Microorganism

The disclosed system uses a microorganism (e.g., probiotic (bacteria, yeast)) to display biomolecules or binding agents.

Generally, one of the criteria that bacteria must meet in order for them to be regarded as probiotics is that they have to be able to survive and thrive throughout the GIT conditions and confer their beneficial effects. A preferred microorganism does not colonize the gut, and is thus, easily controlled.

For example, the microorganism can be Escherichia coli Nissle 1917 (EcN), DH10B, or E. coli MG1655. Other microorganisms that can engineered to incorporate sensors can be probiotics known in the art (see e.g., Bober Synthetic Biology Approaches to Engineer Probiotics and Members of the Human Microbiota for Biomedical Applications. Annu Rev Biomed Eng. 2018; 20:277-300; Mathipa and Thantsha Gut Pathog (2017) 9:28). Probiotics can include bacteria from the genera Streptococcus, Enterococcus, Pediococci, Weissella, or Lactococcus, but the most common ones used belong to Lactobacillus and Bifidobacteria spp.

The microorganism can be yeast, such as a Saccharomyces (e.g., Saccharomyces cerevisiae, Saccharomyces boulardii CNCM 1-745). Due to its inability to colonize the human gut, S. boulardii can be engineered to act as a sensor (and can pass through a host gut).

Binding Agents/Biomolecules

Binding agents, such as biomolecules (e.g., a ligand-binding moiety) can be displayed on the surface of the engineered microorganism. As such, the engineered microorganism can be designed to have a binding affinity to a specific aromatic compound with discriminating specificity.

The binding moiety can be the proteins, TrpR (Trp) sensor, the TyrR (Phe and Tyr) sensor, and the TynA-FeaR (aromatic amine) sensor system. Here, it is shown that their sensitivity can be tuned by rationally selecting and individually mutating amino acids in TyrR, TynA, and FeaR. These sensors have been shown to be selective for Phe, Tyr, PEA, or Tyra.

K_{A} is a half maximal constant. K_{A} values can be greater than 0 mM, between about 0.001 mM and 0.3 mM. For example, for a TyrR in human intestines, serum, or urine a K_{A} of 0.05-0.3 mM is considered sufficient. As another example, in plasma or food, a K_{A} of 0.001-0.1 mM is considered sufficient.

TrpR (Trp) Sensor.

First, multiple engineered TrpR sensors were characterized, which were previously created to be orthogonal to the wild-type E. coli-native system, and assess how the mutations impact the selectivity of the sensors.

Transcriptional Regulatory Protein TyrR (Phe and Tyr) Sensor.

The ligand selectivity of the TyrR sensor was engineered and their sensitivity was tuned by rationally selecting and individually mutating amino acids in TyrR.

As shown in Example 1, the Phe-specific sensors have the potential to be applied to kinetically diagnose and treat disorders that cause Phe-dysregulation without interference from intestinal Tyr.

TyrR protein is involved in transcriptional regulation of aromatic amino acid biosynthesis and transport. TyrR modulates the expression of at least 8 unlinked operons. Seven of these operons are regulated in response to changes in the concentration of the three aromatic amino acids (phenylalanine, tyrosine and tryptophan). These amino acids are suggested to act as co-effectors which bind to the TyrR protein to form an active regulatory protein. In most cases TyrR causes negative regulation, but positive effects on the tyrP gene have been observed at high phenylalanine concentrations. The native tyrR gene (E. coli) is autogenously regulated by a mechanism that gives similar rates of expression of tyrR irrespective of the concentration of the aromatic amino acids.

Monoamine Oxidase TynA-FeaR Regulator (Aromatic Amine) Sensor System.

The ligand selectivity of the TynA-FeaR sensor systems was engineered and sensitivity was tuned by rationally selecting and individually mutating amino acids in TynA and FeaR.

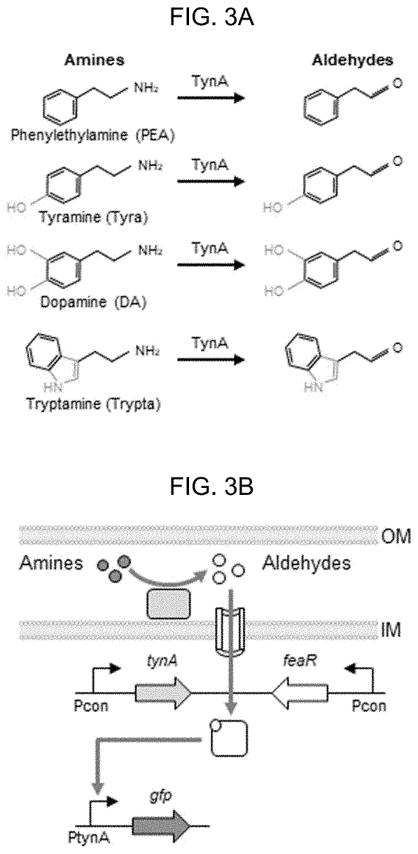

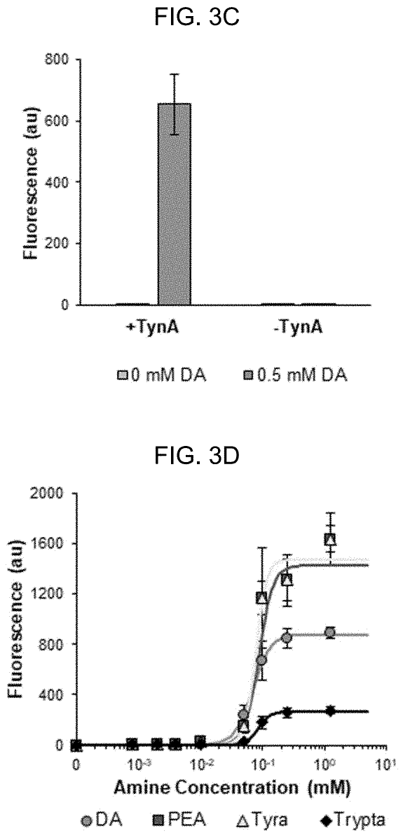

TynA-FeaR system: a sensor plasmid, which consisted of constitutively expressed TynA and FeaR from E. coli MG1655, and a reporter plasmid, which consisted of GFP under the control of PtynA promoter (see e.g., FIG. 3B). Either TynA or FeaR can be engineered for selectivity.

Target Aromatic Compounds

As described herein, the present disclosure provides for a biosensor that specifically interacts with aromatic compounds.

For example, the aromatic compound can be an aromatic amino acid selected from phenylalanine (Phe), tyrosine (Tyr), or tryptophan (Trp).

As another example, the aromatic compound can be an aromatic amine neurochemical selected from dopamine (DA), phenylethylamine (PEA), tyramine (Tyra), tryptamine (Trypta), serotonin, epinephrine, or norepinephrine.

Reporters for Artificial Sensors/Signaling System

The engineered microorganism can be engineered to incorporate reporters for use in artificial sensing and signaling.

The engineered microorganisms described herein can comprise a reporter (or sensor). The reporter can be a sensor protein to detect pH, an immune receptor, or reporter proteins (e.g., GFP, RFP (e.g., mCherry), BFP, luminescence protein (e.g., luciferase)).

For example, the engineered microorganism can comprise a promoter-reporter system and/or a dual-control knob system.

Biosensors/Detectors

The present disclosure also provides for a method of constructing engineered microorganism biosensors that selectively recognize and react to a wide range of sensible target aromatic compounds in vivo. The engineered microorganism can include a reporter. The three sensor systems described here include the TrpR (Trp) sensor, the TyrR (Phe and Tyr) sensor, and the TynA-FeaR (aromatic amine) sensor system.

As described herein, the engineered microorganisms can be used for sensing applications. For example, a portion of a functional protein can be used to induce or repress a transcriptional response.

The promoter PtyrP upregulates gene expression via the phenylalanine-binding transcriptional dual regulator TyrR.

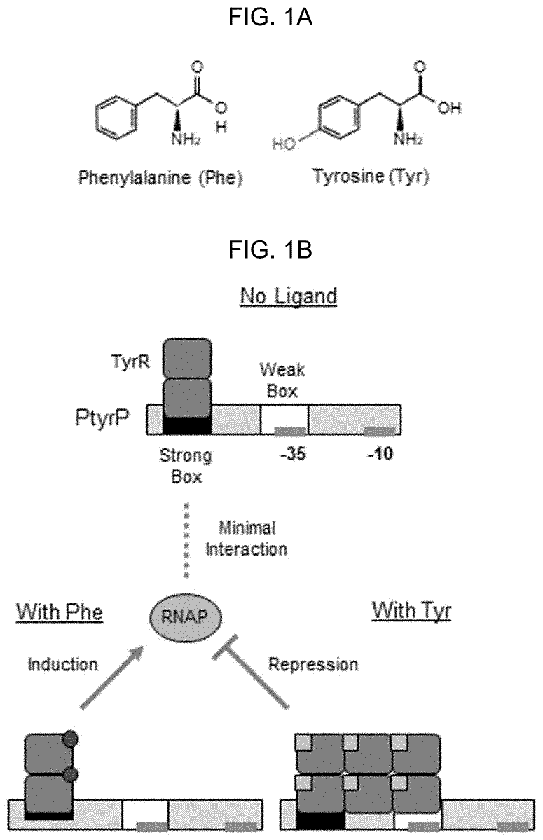

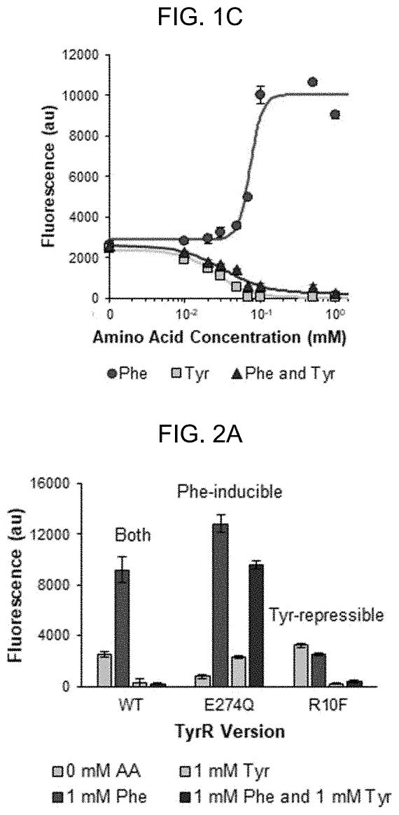

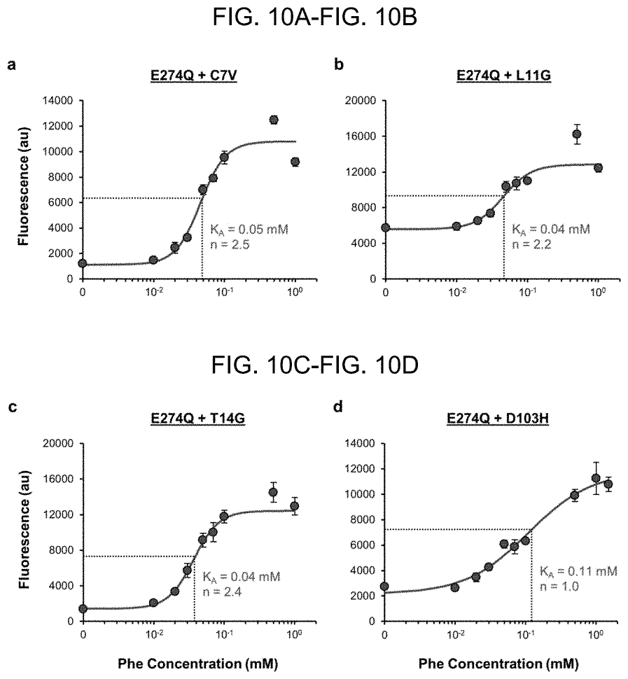

As described in Example 1, six E. coli-native promoters were characterized using genomic expression or plasmid-based overexpression of TyrR to identify the best promoter in EcN. PtyrP was selected as a sensor as it displayed an activating response to Phe and a repressing response to Tyr, allowing for clear differentiation between the presence of each amino acid, especially with overexpression of TyrR (see e.g., FIG. 1B and FIG. 10). Other promoters tested included PtynA, PtyrP, PtyrR, Pmtr, PtyrB, ParoF, and ParoP.

As described herein, engineered microorganisms that can be used for detecting or sensing aromatic compounds. For example, a detection moiety can be used (e.g., GFP, RFP, BFP, luminescence protein). As such, every engineered microorganism that sensed a target aromatic compound will overexpress (“light up”) or be repressed.

As a microbial biosensor targeting gut health, the disclosed engineered E. coli can overcome limitations of conventional approaches that depend on very limited existing sensing mechanisms in nature.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:

(i) By disabling repressors. The gene is expressed because an inducer binds to the repressor. The binding of the inducer to the repressor prevents the repressor from binding to the operator. RNA polymerase can then begin to transcribe operon genes.

(ii) By binding to activators. Activators generally bind poorly to activator DNA sequences unless an inducer is present. An activator binds to an inducer and the complex binds to the activation sequence and activates the target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation. A “wild type” organism can be genetically engineered to modulate native or non-native gene expression resulting in a transgenic or engineered organism.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=XN100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity to a reference sequence (e.g., binding pocket, entire protein, or functional fragment thereof) can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, expression of various signals (e.g., mutants, proteins) can be modulated (e.g., reduced, eliminated, or enhanced) using genome editing. Processes for genome editing are well known; see e.g. Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of various signals by genome editing can result in engineered sensors for detection of aromatic compounds, for example.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for targeting cells by the removal or addition of various signals (e.g., to activate (e.g., CRISPRa), upregulate, downregulate).

For example, the methods as described herein can comprise a method for altering a target polynucleotide sequence in a cell comprising contacting the polynucleotide sequence with a clustered regularly interspaced short palindromic repeats-associated (Cas) protein.

Examples of engineering a probiotic, for example, can include inserting a functional gene with a viral vector.

Any vector known in the art can be used. For example, the vector can be a viral vector selected from retrovirus, lentivirus, herpes, adenovirus, adeno-associated virus (AAV), rabies, Ebola, lentivirus, or hybrids thereof.

### Gene Editing Strategies

| Viral Vectors | Strategy |
|---|---|
| Retroviruses | Retroviruses are RNA viruses transcribing their single- |
| | stranded genome into a double-stranded DNA copy, |
| | which can integrate into host chromosome |
| Adenoviruses (Ad) | Ad can transfect a variety of quiescent and proliferating |
| | cell types from various species and can mediate |
| | robust gene expression |
| Adeno-associated | Recombinant AAV vectors contain no viral DNA and can |
| Viruses (AAV) | carry ~4.7 kb of foreign transgenic material. They |
| | are replication defective and can replicate only while |
| | coinfecting with a helper virus |
| plasmid DNA | pDNA has many desired characteristics as a gene |
| (pDNA) | therapy vector; there are no limits on the size or genetic |
| | constitution of DNA, it is relatively inexpensive to supply, |
| | and unlike viruses, antibodies are not generated |
| | against DNA in normal individuals |
| RNAi | RNAi is a powerful tool for gene specific silencing that |
| | could be useful as an enzyme reduction therapy or |
| | means to promote read-through of a premature stop |
| | codon |

Aromatic Compound-Associated Diseases, Disorders, or Conditions

The sensors, systems, and methods described herein can be used for the detection or monitoring of aromatic compound-associated diseases, disorders, or conditions.

For example, elevated levels of structurally similar Phe and Tyr are associated with the distinct disorders phenylketonuria and type 2 tyrosinemia, respectively. As another example, extreme levels of PEA have been associated with a variety of psychological disorders, the presence of Tyra leads to catecholamine release and an increase in blood pressure, and the presence of Trypta causes serotonin release and the stimulation of gastrointestinal motility. Additionally, the presence of PEA, Tyra, and Trypta in food are indicators of microbial contamination, and eating foods with high levels of Tyra can lead to poisoning. Currently, there are no biosensors with high ligand specificity for these chemicals.

Serotonin is a chemical that the body produces naturally. It's needed for the nerve cells and brain to function. But too much serotonin can cause signs and symptoms that can range from mild (shivering or diarrhea) to severe (muscle rigidity, fever, or seizures). Severe serotonin syndrome can cause death if not treated. Serotonin syndrome can be a result of too much serotonin in your body. It is usually caused by taking drugs or medications that affect serotonin levels. Stopping the drug(s) or medication(s) causing serotonin syndrome is the main treatment.

Many of these metabolites, including DA, PEA, Tyra, and Trypta can be found in the intestines. As such, selective detection of such compounds is needed.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to engineered probiotics, engineered protein regulators, and components for making and using same. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject, a group of healthy subjects, or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Engineering Ligand-Specific Biosensors for Aromatic Amino Acids and Neurochemicals

This work represents a considerable achievement and includes the first ligand-specific or ligand-selective sensors for aromatic amino acids such as phenylalanine (Phe) and tyrosine (Tyr) as well as neurochemicals such as phenylethylamine (PEA) and tyramine (Tyra), which are structurally similar and have been implicated in a variety of medical conditions. Importantly, it lays the groundwork for developing medically relevant probiotic sensors with high specificity, which is critical to differentiate metabolites with divergent functions and create smart probiotics for accurate diagnostic tools.

Microbes have evolved diverse sensor systems that detect metabolites of great medical importance. These sensors have the potential to be utilized in probiotic microbes to provide diagnostic information and deliver therapeutics with temporal and geographical precision. However, microbial sensors found in nature often have promiscuity to several structurally similar aromatic amino acids, common neurotransmitters, or other neuromodulators, limiting their practical applications. Although similar, these metabolites have significantly different functions. For example, chronically elevated levels of structurally similar Phe and Tyr are associated with the distinct disorders phenylketonuria and type 2 tyrosinemia, respectively. Extreme PEA levels have been associated with a variety of psychological disorders, while the presence of Tyra leads to catecholamine release and an increase in blood pressure. Due to such differences in associated diseases and functions, specific sensing is critical for probiotic sensor applications. Despite advances in protein engineering for specific ligand-protein interactions, engineering ligand-specific sense-and-respond systems remain challenging, especially when the target ligands are structurally similar and when ligand-protein binding should control downstream functions such as gene expression. This is mainly due to the challenge in coupling subtle protein conformational changes caused by binding of similar ligands with differential DNA interactions.

In this work, two promiscuous sensors are characterized that recognize aromatic metabolites associated with various metabolic and neurological disorders. Common methods of protein engineering require extensive structural knowledge of the proteins or massive library sizes. In contrast, to improve ligand selectivity, the responsible proteins, including TyrR, TynA, and FeaR, were rationally engineered by identifying and individually mutagenizing specific amino acids in and around the ligand-binding sites of these sensors. From these three case studies, this simple and generalizable method of protein engineering is shown to be effective and time-efficient, require small library sizes with only a basic understanding of the protein structure, and enables changes in ligand-protein binding specificity while maintaining protein-DNA interaction and thus downstream gene expression control.

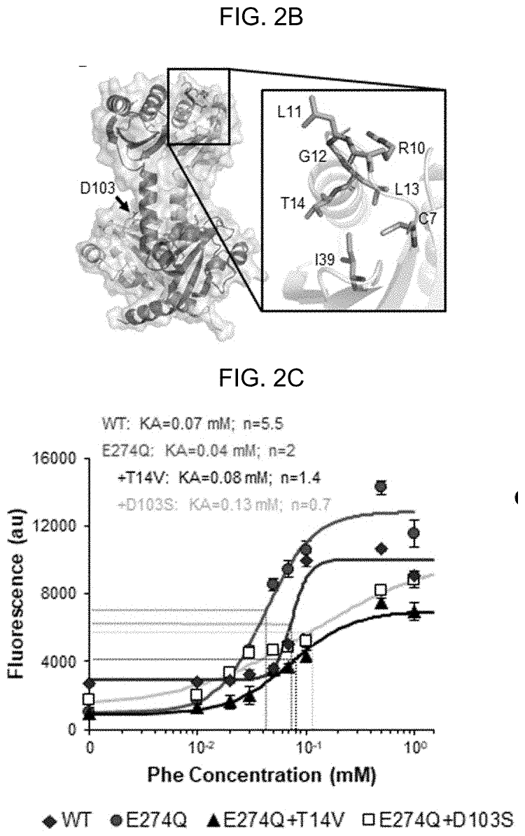

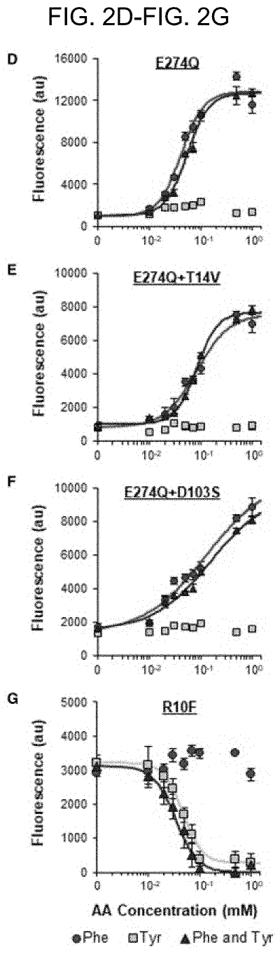

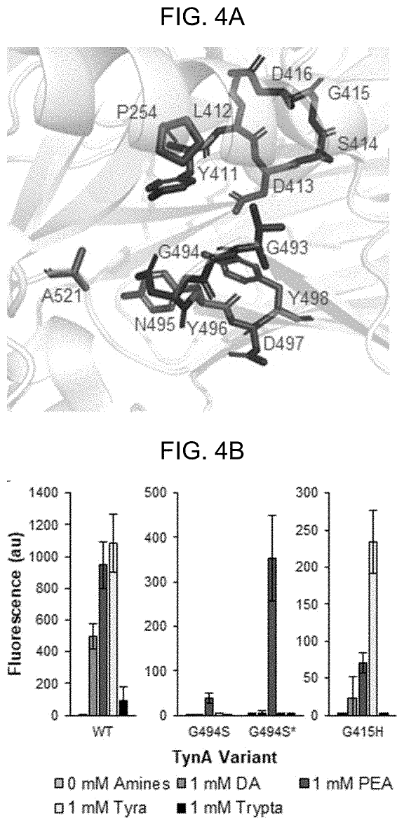

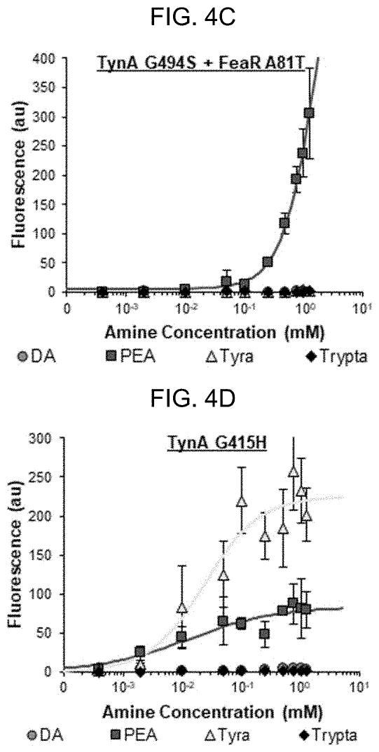

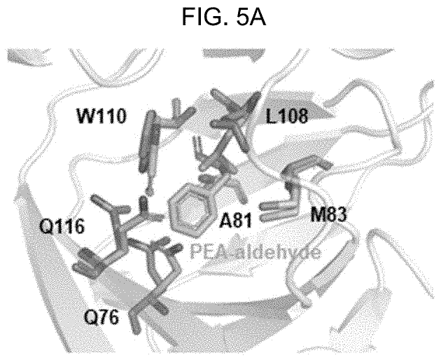

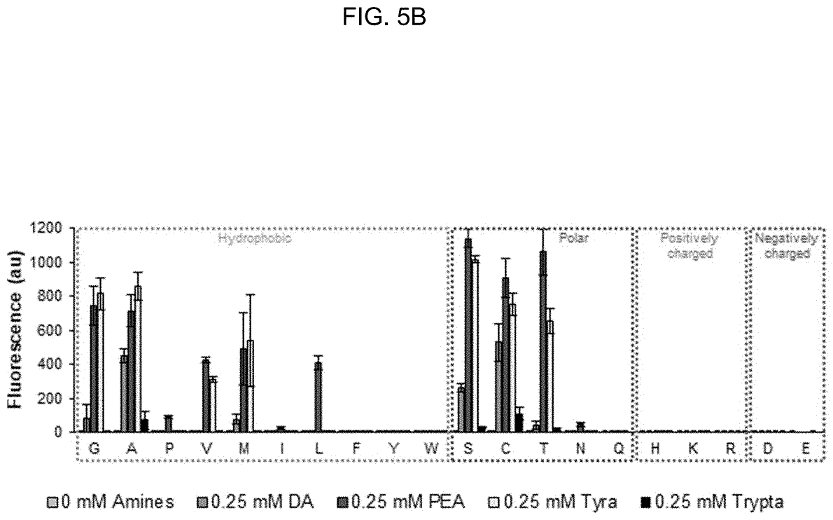

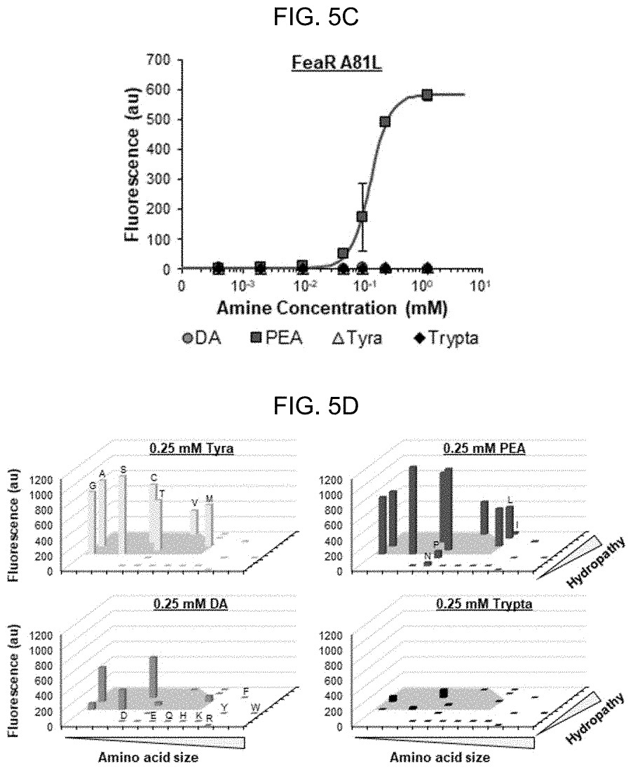

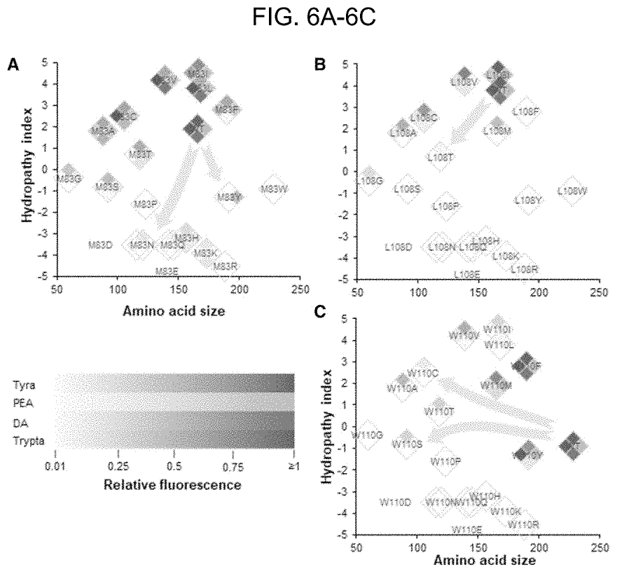

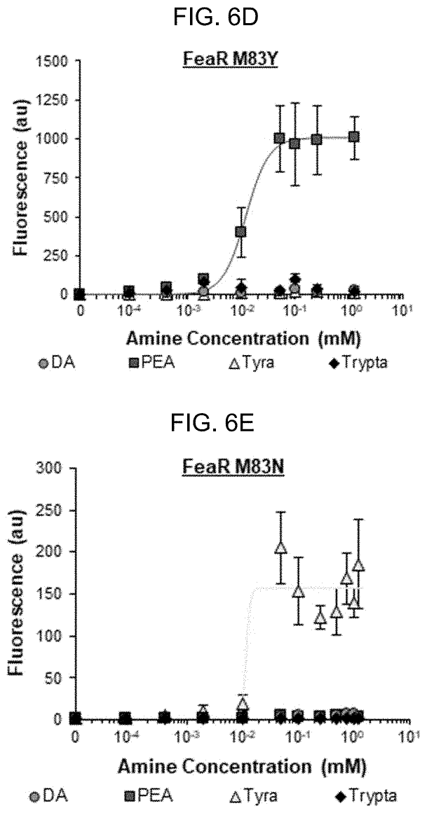

Protein engineering for ligand-protein interaction has been extensively performed. However, linking ligand-protein binding to output response remains challenging. Current approaches include designing proteins that fluoresce upon ligand binding or employing ligand-binding transcription factors (TFs). While the former can be used to create sensors, the latter can generate sensors or signal-responsive controllers for gene expression. Because this TF-based system requires the maintenance or engineering of DNA-protein interaction in addition to engineering ligand-protein binding, engineering the TF-based system has been challenging, especially when the target ligands are structurally similar. To address this issue, demonstrated herein is an approach to change ligand-TF binding specificity by leveraging differential multimerization patterns of TyrR without affecting DNA-TF binding interaction (see e.g., FIG. 1A-FIG. 1C and FIG. 2A-FIG. 2G), and a “dual-control knob” strategy to improve the specificity and sensitivity of substrate-enzyme and ligand-TF interactions while maintaining DNA-TF binding interactions (see e.g., FIG. 3A-FIG. 3D, FIG. 4A-FIG. 4D, see e.g., FIG. 5A-FIG. 5D, and FIG. 6A-FIG. 6E).

The ligand binding site for TynA comprises at least a functional portion of SEQ ID NO: 32, having catalytic residues D413 and Y496. The ligand binding site for FeaR comprises at least a functional portion of SEQ ID NO: 36, wherein the ligand binding region is from W12 to S118 cover by the magenta region in FIG. 20. A81, M83, L108, W110 are putative binding residues tested and Q76, Q116 are two other predicted binding residues (FIG. 5A & FIG. 20).

In addition, the computational and experimental analyses of FeaR provide novel insights into the otherwise uncharacterized structure of FeaR. The location of the FeaR ligand-binding pocket and potentially critical residues in ligand binding are identified herein for the first time. This approach allows for the generation of a highly efficient, specific, and sensitive sensor for PEA. In addition, residues that can be mutagenized in future work to generate novel ligand-selective sensors for several additional aromatic neurotransmitters and neuromodulators are also identified herein.

The novel ligand-selective sensors generated in this work will have diverse applications in synthetic biology. They can be applied to engineer autonomous microbes that dynamically identify microbial contamination in consumable products, manage various debilitating neurological disorders, and normalize dysregulated metabolites associated with metabolic disorders. In addition, the protein engineering methods demonstrated here can be widely applied to develop enzymes and sensors with applications in bioenergy, commodity chemicals, bioremediation, and healthcare.

Summary

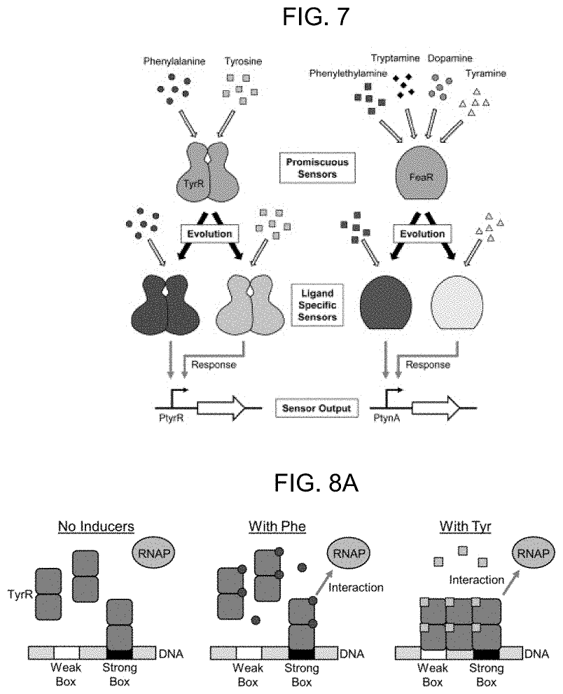

Microbial biosensors have diverse applications in metabolic engineering and medicine. Specific and accurate quantification of chemical concentrations allows for adaptive regulation of enzymatic pathways and temporally precise expression of diagnostic reporters. Although biosensors should differentiate structurally similar ligands with distinct biological functions, such specific sensors are rarely found in nature and are challenging to create. Using E. coli Nissle 1917, generally regarded as a safe microbe, two biosensor systems that promiscuously recognize aromatic amino acids or neurochemicals were characterized. To improve the sensors' selectivity and sensitivity, rational protein engineering was applied by identifying and mutagenizing amino acid residues and the ligand-specific biosensors for phenylalanine, tyrosine, phenylethylamine, and tyramine were successfully demonstrated (see e.g., FIG. 7). Additionally, the approach revealed insights into the uncharacterized structure of the FeaR regulator, including critical residues in ligand binding. These results lay the groundwork for developing kinetically adaptive microbes for diverse applications.

Introduction

Microbial biosensors can be utilized to kinetically regulate and quantify the products of metabolic pathways, diagnose diseases in vivo in probiotics, and analyze ex vivo samples through wearable, paper-based, and cell-free systems. Synthetic biological sensors can be created using several protein design approaches. However, sensors can also be obtained more efficiently by mining sensors that naturally exist in microbes. These sensors can often be directly transferred into probiotic organisms or purified for cell-free sensor applications. However, most natural sensors recognize multiple structurally similar, but functionally diverse, metabolites often found in close proximity. To accurately correlate the concentration of a chemical with biological outcomes, sensors need to differentiate between different chemicals and recognize the relevant chemical with the precise sensitivity.

Specific sensing is critical for many microbial sensor applications. A variety of protein engineering methods have been demonstrated for optimizing the selectivity and sensitivity of protein and RNA sensors. These approaches include directed evolution and rational design, and computational de novo design. Although each method has advantages, rational engineering can uniquely be performed using both basic knowledge of the protein structure and small library sizes, allowing for rapid construction and screening of libraries. In addition, when the structure of the protein is unknown, conserved or essential residues can also be identified through computational simulations or by aligning the sequence of the sensor with other proteins in the same family. Despite advances in protein engineering, creating ligand-specific sense-and-respond systems remains challenging due to the need to couple subtle protein conformational changes with differential protein-DNA interactions and gene expression control, especially when the target ligands are structurally similar.

The aromatic amino acids phenylalanine (Phe) and tyrosine (Tyr) are common microbial metabolic engineering products and precursors derived from the same pathway. In addition, chronically elevated levels of structurally similar Phe and Tyr are associated with the distinct disorders phenylketonuria and type 2 tyrosinemia, respectively (see e.g., TABLE 1).

TABLE 1
Concentrations of phenylalanine, tyrosine, phenylethylamine,
and tyramine from different sources.

| Chemical | Source | Concentration (mM) |
|---|---|---|
| Phenylalanine | Ileum, healthy | 0.30 |
| | Plasma, healthy | 0.06 |
| | Plasma, phenylketonuria | 1.1 |
| | Serum, healthy | 0.06-0.07 |
| | Urine, healthy | 0.06 |
| Tyrosine | Ileum, healthy | 0.26 |
| | Plasma, healthy | 0.07 |
| | Plasma, tyrosinemia | 1.3 |
| | Serum, healthy | 0.06-0.08 |
| | Urine, healthy | 0.04 |
| Phenylethylamine | Sauerkraut, safe limit | 0.03 |
| | Plasma, healthy | 0.04 |
| | Plasma, schizophrenics | 0.10 |
| Tyramine | Sauerkraut, safe limit | 0.12 |
| | Plasma, healthy | 0.0007 |
| | Plasma, elevated blood pressure | >0.001 |

Sensors for both metabolites have been generated in different E. coli strains. However, the sensors are based on the wild-type version of the multi-ligand responsive TyrR transcription factor (TF) from E. coli and have limited selectivity and low dynamic ranges. Similarly, the structurally similar amines phenylethylamine (PEA), tyramine (Tyra), and tryptamine (Trypta) are all commonly found in food and in the gut but contribute to distinct biological outcomes (see e.g., TABLE 1). For example, extreme levels of PEA have been associated with a variety of psychological disorders, the presence of Tyra leads to catecholamine release and an increase in blood pressure, and the presence of Trypta causes serotonin release and the stimulation of gastrointestinal motility. Additionally, the presence of PEA, Tyra, and Trypta in food are indicators of microbial contamination, and eating foods with high levels of Tyra can lead to poisoning. Currently, there are no biosensors with high ligand specificity for these chemicals.

Described herein is the generation of sensors for the aromatic metabolites Phe, Tyr, PEA, and Trypta using Escherichia coli Nissle 1917 (EcN) as a host. To generate sensors for these aromatic metabolites, two sensor systems were identified and engineered: the TyrR (Phe and Tyr) sensor and the TynA-FeaR (aromatic amine) sensor system. The ligand selectivity of the TyrR and TynA-FeaR sensor systems was engineered and their sensitivity was tuned by rationally selecting and individually mutating amino acids in TyrR, TynA, and FeaR. This method of rational protein engineering quickly generates multiple small libraries that can be efficiently screened, making it an attractive approach for specificity engineering. Altogether, the sensors specific for Phe, Tyr, PEA, or Tyra were generated. In engineering FeaR, insights were provided into the uncharacterized structure of FeaR and residues important for ligand binding were identified. Herein are provided sensors with diverse applications in microbial biosensing as well as a generalizable approach to modulating the specificity of ligand-protein binding while maintaining protein-DNA interactions and thus downstream gene expression control.

Results

Developing Specific Phenylalanine and Tyrosine Sensors

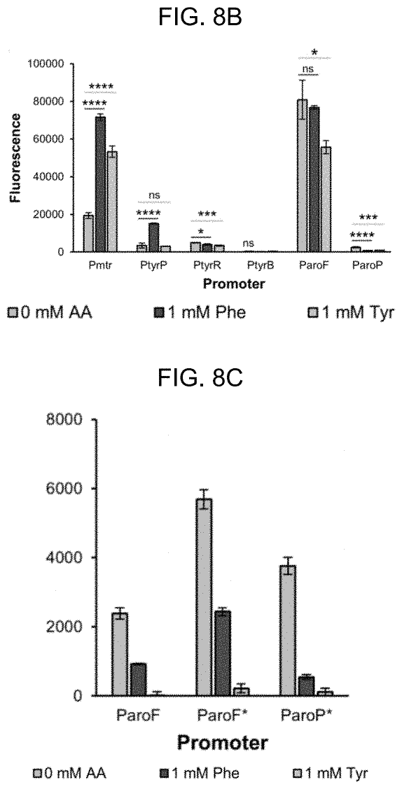

Phe and Tyr are structurally similar metabolites utilized throughout the body for many processes as additives in food and animal feed and as precursors for many chemicals and pharmaceuticals (see e.g., FIG. 1A). Notably, two distinct disorders (phenylketonuria and tyrosinemia) cause a buildup of dietary Phe or Tyr, necessitating sensors that can differentiate between the two amino acids. Microbial TyrR regulates its target promoters in both a Phe- and Tyr-dependent manner (see e.g., FIG. 1B and FIG. 8A). In the absence of either amino acid, TyrR forms homodimers that bind to high-affinity recognition sites, or strong boxes. When bound to Phe, the dimers interact with RNA polymerase to influence transcription initiation. When bound to Tyr, TyrR forms homohexamers that bind to both the strong box and the adjacent low-affinity weak box and interact with RNA polymerase. When the binding sites are located upstream of the −35 site of the promoter, these interactions increase expression of the downstream gene. When the binding sites are overlapping or downstream of the −35 site, TyrR instead represses expression. Given the complexity of DNA-TyrR binding and ligand-TyrR binding modes, a stepwise approach was used to develop ligand-specific sensors with the hypothesis that targeted engineering of TyrR would enable its differential ligand binding without affecting DNA-binding interactions.

First, six E. coli-native promoters were characterized using genomic expression or plasmid-based overexpression of TyrR to identify the best promoter in EcN (see e.g, FIG. 8B and FIG. 8C). PtyrP was selected as the sensor as it displayed an activating response to Phe and a repressing response to Tyr, allowing for clear differentiation between the presence of each amino acid, especially with overexpression of TyrR (see e.g., FIG. 1B and FIG. 10). The PtyrP promoter has a strong box upstream of the −35 site and a weak box overlapping the −35 site, allowing TyrR to induce expression in the presence of Phe and repress expression in the presence of Tyr. In addition, PtyrP showed the highest fold induction in the presence of Phe (4-fold) compared with all tested promoters and strong repression in the presence of Tyr (59-fold). Notably, high expression levels of TyrR are required for Tyr-dependent repression of PtyrP, as shown by the lack of repression when only controlled by genomic TyrR expression (see e.g., FIG. 8B). This lack of response gives PtyrP a false appearance of Phe-specificity when paired with low TyrR expression levels. However, TyrR regulated the promoter in a Tyr-dominant manner. In the presence of Tyr and overexpression of TyrR, TyrR repressed PtyrP independent of the presence of Phe (see e.g., FIG. 10).

The E274Q TyrR mutation was previously shown to prevent TyrR from forming hexamers, which occurs when TyrR is bound to Tyr. Since hexamerization is required for Tyr-mediated repression of PtyrP, this mutation should render TyrR nonresponsive to Tyr but maintain Phe-dependent induction. When the mutation was inserted into the plasmid-expressed TyrR, Tyr-dependent repression of PtyrP was mitigated and the Tyr-dominating response of the TF was eliminated (see e.g., FIG. 2A). In addition, the mutant had an improved fold induction in response to Phe (16-fold). This is the highest dynamic range demonstrated by a Phe sensor to date.

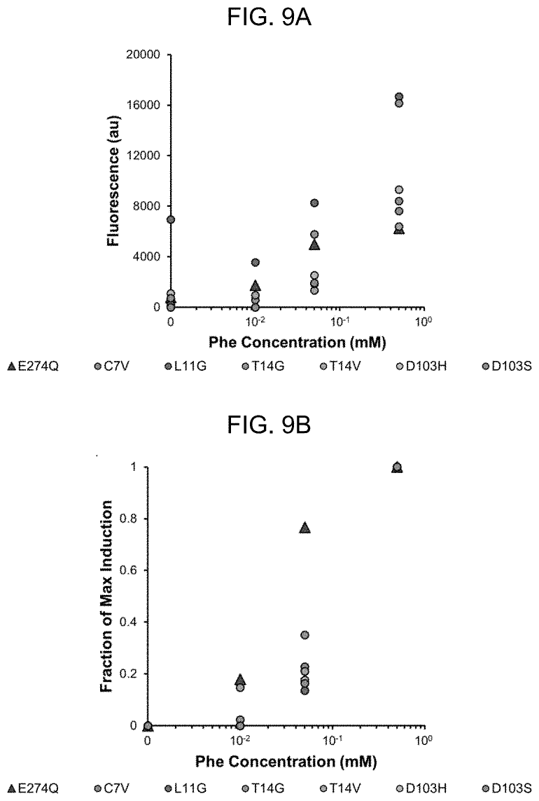

Next, the Phe-sensitivity of the E274Q mutant was tuned to create sensors that better align with variable physiological concentrations and improve the utility of the sensors for therapeutic and diagnostic applications. Eight amino acid positions with known roles in TyrR-mediated promoter induction were selected (see e.g., FIG. 2B). Each amino acid has been shown to be important, but potentially non-essential, for Phe binding. When the transfer curve of the parent E274Q sensor was obtained, half-maximal induction was observed at 0.04 mM Phe (see e.g., FIG. 2C and FIG. 2D), lower than the Phe concentrations in human intestines, serum, and urine (see e.g., TABLE 1, 0.05-0.3 mM). Thus, saturation mutagenesis was performed on each amino acid individually and the small, targeted libraries were screened for a reduced sensitivity to Phe (see e.g., FIG. 9). Three variants displayed reproducibly lower sensitivities: E274Q+T14V, E274Q+D103H, and E274Q+D103S with K_{A} values of 0.08, 0.11, and 0.13 mM, respectively (see e.g., FIG. 2C and FIG. 10A-FIG. 10D). The E274Q+T14V and E274Q+D103S variants were selected for further characterization as they displayed the widest range of sensitivities and maintained the highest fold-inductions. Both mutants displayed consistent sensitivities to Phe in the presence and absence of Tyr and had no apparent response to Tyr alone (see e.g., FIG. 2E and FIG. 2F). When both mutations were combined in E274Q TyrR, the response to Phe was eliminated. These Phe-specific sensors have the potential to be applied to kinetically diagnose and treat disorders that cause Phe-dysregulation without interference from intestinal Tyr.

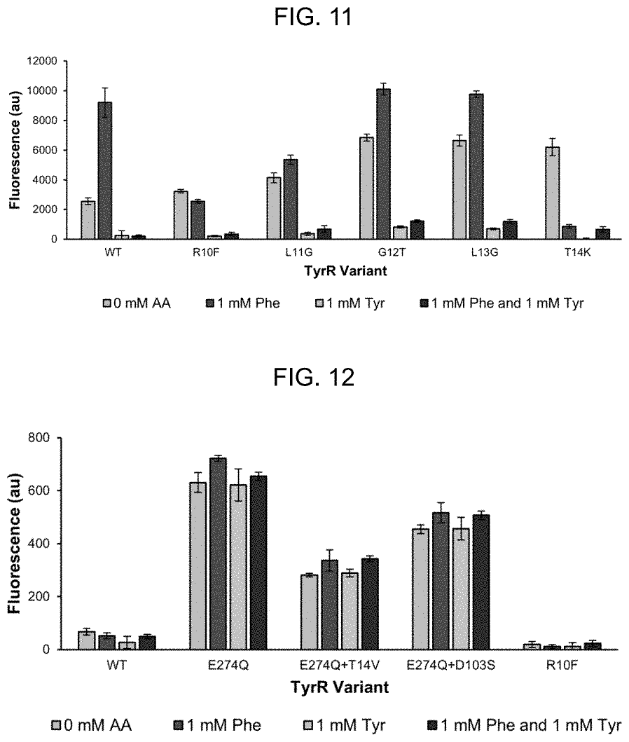

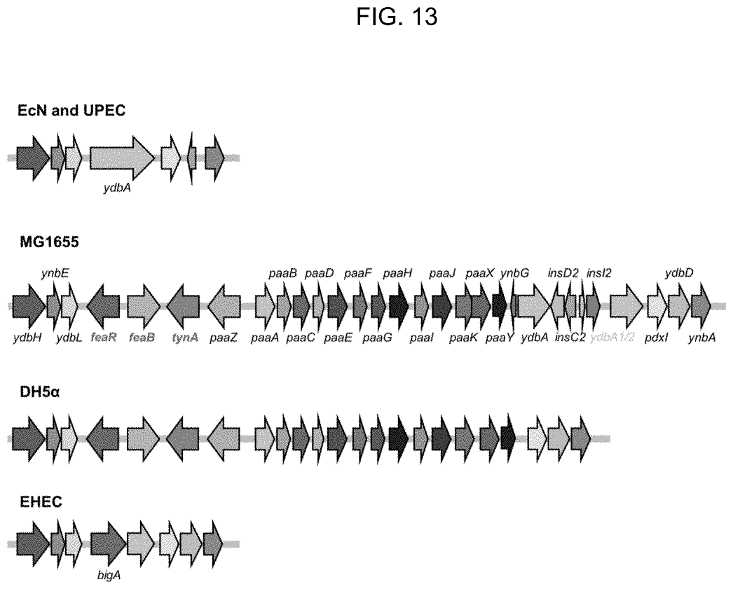

TyrR was also engineered for Tyr specificity. Because TyrR binds to Phe and Tyr using two separate binding pockets, the libraries designed to identify less sensitive Phe sensors were used to identify variants completely devoid of Phe-activity, while maintaining Tyr activity (see e.g., FIG. 11). The most selective mutant, R10F, demonstrated 15-fold repression in the presence of Tyr independent of the presence of Phe (see e.g., FIG. 2A). The positively charged R10 residue is positioned near the region where the ligand's carboxyl group could stabilize Phe binding. However, conversion to a hydrophobic F10 may instead prevent this stabilization. In addition, no significant response to Phe alone was observed. When the transfer curve of the mutant was characterized, K_{A} of 0.05 mM in response to Tyr was identified (see e.g., FIG. 2G). In addition, it was demonstrated that rational saturation mutagenesis can effectively generate functionally diverse, specific sensors from one promiscuous TF. As expected for all chemical sensors, one caveat of these Phe- and Tyr-specific sensors is that they cannot differentiate elevated concentrations of the two ligands in rich medium, which has saturating concentrations of the two amino acids (see e.g., FIG. 12).

Engineering Ligand-Specific Aromatic Amine Sensors

The body harbors a variety of aromatic amines with diverse neurological functions. Many of these metabolites, including dopamine (DA), PEA, Tyra, and Trypta, can be found in the intestines. Many commensal microbes can convert these amines into aldehydes through monoamine oxidases (see e.g., FIG. 3A). These aldehydes can be further catabolized to carboxylic acids by aldehyde dehydrogenases. In some E. coli strains, expression of the monoamine oxidase TynA and aldehyde dehydrogenase FeaB are regulated by the AraC-type FeaR TF, which was shown to induce expression of TynA and FeaB in the presence of multiple aldehydes formed from the corresponding amines. This tynA-feaB-feaR gene cluster was found to be present in select commensal E. coli strains, but not in EcN (see e.g., FIG. 13). This promiscuous sensing system provides an opportunity to sense a variety of important aromatic amines using engineered microbes. However, since each amine has a different function, it is essential that the microbe can differentiate the metabolites.

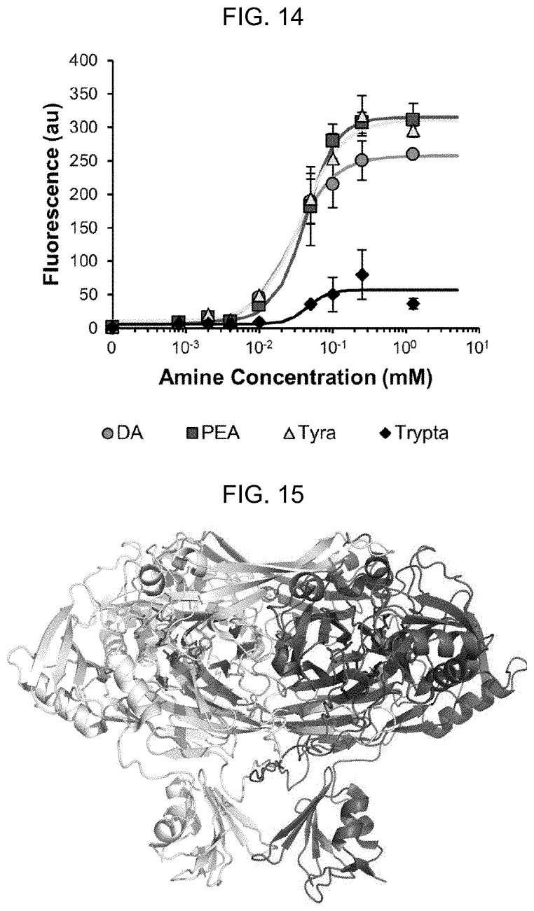

To develop sensors for aromatic amines, the TynA-FeaR system was first characterized in EcN. A sensor plasmid was constructed, which consisted of constitutively expressed TynA and FeaR from E. coli MG1655, and a reporter plasmid, which consisted of GFP under the control of PtynA (see e.g., FIG. 3B). Next, it was confirmed that the system induces expression of PtynA in response to aromatic aldehydes, not amines (see e.g., FIG. 3C). In the presence of TynA, the addition of 0.5 mM DA resulted in a 410-fold increase in GFP expression. In contrast, the induction was eliminated when TynA was inactivated. Next, the sensitivity and selectivity to different ligands was characterized. The system displayed the strongest response to the addition of PEA or Tyra, inducing expression of the reporter by more than 1,500-fold with the addition of 1.25 mM of each amine (see e.g., FIG. 3D and FIG. 14). The addition of DA resulted in the next highest response, with approximately 900-fold induction. Trypta had the weakest response, inducing expression by approximately 280-fold. The system showed a similar sensitivity to each ligand, with half-maximal induction occurring at 0.07-0.09 mM (see e.g., TABLE 2).

TABLE 2
Fitted Hill equation parameters for the specified figures.

| | | | | | | | K_{A} | |
|---|---|---|---|---|---|---|---|---|
| FIG. | Strain | Plasmid(s) | Ligand | Fₘₐₓ | Fₘᵢₙ | n | (mM) | RMSE |
| 1C | sAGR039 | pAGR010 | Phenylalanine | 10500 | 2900 | 5.5 | 0.073 | 20.9 |
| 1C | sAGR039 | pAGR010 | Tyrosine | 2470 | 38.1 | 1.9 | 0.026 | 8.5 |
| 1C | sAGR039 | pAGR010 | Phenylalanine | 2640 | 255 | 1.5 | 0.032 | 10.7 |
| | | | and tyrosine | | | | | |
| 2C, 2D | sAGR039 | pAGR102 | Phenylalanine | 12800 | 982 | 2 | 0.042 | 690 |
| 2D | sAGR039 | pAGR102 | Phenylalanine | 12700 | 1060 | 2.1 | 0.055 | 442 |
| | | | and tyrosine | | | | | |
| 2C, 2E | sAGR039 | pAGR157 | Phenylalanine | 7560 | 814 | 1.4 | 0.083 | 273 |
| 2E | sAGR039 | pAGR157 | Phenylalanine | 7670 | 1030 | 2.1 | 0.084 | 178 |
| | | | and tyrosine | | | | | |
| 2C, 2F | sAGR039 | pAGR159 | Phenylalanine | 10700 | 1310 | 0.7 | 0.128 | 338 |
| 2F | sAGR039 | pAGR159 | Phenylalanine | 9710 | 1510 | 0.8 | 0.155 | 212 |
| | | | and tyrosine | | | | | |
| 2G | sAGR039 | pAGR473 | Tyrosine | 3210 | 279 | 2.3 | 0.047 | 7.7 |
| 2G | sAGR039 | pAGR473 | Phenylalanine | 3110 | 3.9 | 2 | 0.034 | 10.2 |
| | | | and Tyrosine | | | | | |
| 3D | sAGR039 | pCX008, | Dopamine | 881 | 0.1 | 3 | 0.068 | 5.2 |
| | | pHJY022 | | | | | | |
| 3D, S15a | sAGR039 | pCX008, | Phenylethylamine | 1430 | 0 | 3.9 | 0.087 | 5.9 |
| | | pHJY022 | | | | | | |
| 3D | sAGR039 | pCX008, | Tyramine | 1470 | 4.4 | 5 | 0.077 | 9.3 |
| | | pHJY022 | | | | | | |
| 3D | sAGR039 | pCX008, | Tryptamine | 268 | 1.3 | 4.3 | 0.085 | 9.9 |
| | | pHJY022 | | | | | | |
| 4C, 22B | sAGR039 | pCX008, | Phenylethylamine | 790 | 5.4 | 1.4 | 1.8 | 5.2 |
| | | pCX014 | | | | | | |
| 4D | sAGR039 | pCX008, | Phenylethylamine | 84 | 0 | 0.6 | 0.012 | 8.7 |
| | | pCX015 | | | | | | |
| 4D | sAGR039 | pCX008, | Tyramine | 226 | 0 | 0.9 | 0.023 | 25.4 |
| | | pCX015 | | | | | | |
| 5C | sAGR039 | pCX008, | Phenylethylamine | 585 | 4.8 | 2.7 | 0.137 | 4 |
| | | pCX024 | | | | | | |
| 6D | sAGR039 | pCX008, | Phenylethylamine | 1004 | 5.8 | 2.1 | 0.012 | 32.3 |
| | | pCX068 | | | | | | |
| 6E | sAGR039 | pCX008, | Tyramine | 158 | 1.5 | 12 | 0.012 | 21.7 |
| | | pCX073 | | | | | | |
| 10A | sAGR039 | pAGR160 | Phenylalanine | 10800 | 1120 | 2.5 | 0.046 | 790 |
| 10B | sAGR039 | pAGR155 | Phenylalanine | 12900 | 5570 | 2.2 | 0.045 | 1090 |
| 10C | sAGR039 | pAGR156 | Phenylalanine | 12400 | 1430 | 2.4 | 0.037 | 680 |
| 10D | sAGR039 | pAGR158 | Phenylalanine | 11800 | 2150 | 1 | 0.11 | 397 |
| 14 | sAGR039 | pCX008, | Dopamine | 258 | 4.4 | 1.5 | 0.028 | 5.2 |
| | | pHJY022 | | | | | | |
| 14 | sAGR039 | pCX008, | Phenylethylamine | 315 | 10.4 | 2 | 0.042 | 5.9 |
| | | pHJY022 | | | | | | |
| 14 | sAGR039 | pCX008, | Tyramine | 311 | 10.6 | 1.6 | 0.037 | 9.3 |
| | | pHJY022 | | | | | | |
| 14 | sAGR039 | pCX008, | Tryptamine | 57 | 5.9 | 3.1 | 0.045 | 9.9 |
| | | pHJY022 | | | | | | |
| 17A | sAGR039 | pCX008, | Phenylethylamine | 9280 | 1.1 | 1.2 | 33 | 3.2 |
| | | pCX014 | | | | | | |
| 17B | sAGR039 | pCX008, | Phenylethylamine | 118 | 0 | 0.4 | 1.64 | 8.6 |
| | | pCX015 | | | | | | |
| 17B | sAGR039 | pCX008, | Tyramine | 152 | 0 | 0.8 | 0.041 | 16.7 |
| | | pCX015 | | | | | | |
| 18A | sAGR039 | pCX008, | Phenylethylamine | 18 | 0.8 | 36 | 0.727 | 0.8 |
| | | pCX010 | | | | | | |
| 18B, 22B | sAGR039 | pCX008, | Phenylethylamine | 4012 | 3.4 | 1.3 | 43 | 3.9 |
| | | pCX010 | | | | | | |
| 22A | sAGR039 | pCX008, | Phenylethylamine | 1010 | 18 | 4.3 | 0.046 | 2.8 |
| | | pCX016 | | | | | | |
| 23A | sAGR039 | pCX008, | Phenylethylamine | 462 | 3.4 | 1.2 | 3.33 | 5.1 |
| | | pCX032 | | | | | | |
| 25B, 22B | sAGR039 | pCX008, | Phenylethylamine | 475 | 5.9 | 1.5 | 1.34 | 9.1 |
| | | pCX032 | | | | | | |
| 24 | sAGR039 | pCX008, | Phenylethylamine | 289 | 5.1 | 2.6 | 0.074 | 5.8 |
| | | pCX024 | | | | | | |
| 25A | sAGR039 | pCX008, | Phenylethylamine | 14 | 0.4 | 8.4 | 0.014 | 1 |
| | | pCX025 | | | | | | |
| 25B | sAGR039 | pCX008, | Phenylethylamine | 112 | 1.1 | 20.3 | 0.052 | 4.3 |
| | | pCX025 | | | | | | |
| 28A | sAGR039 | pCX008, | Tyramine | 55 | 0.4 | 8.4 | 0.011 | 13.7 |
| | | pCX083 | | | | | | |
| 28B | sAGR039 | pCX008, | Tyramine | 78 | 1.8 | 1.6 | 0.008 | 13.9 |
| | | pCX099 | | | | | | |
| 28C | sAGR039 | pCX008, | Tyramine | 127 | 1.5 | 9.8 | 0.027 | 19.7 |
| | | pCX116 | | | | | | |
| 28D | sAGR039 | pCX008, | Tyramine | 148 | 0.9 | 9.7 | 0.011 | 13.6 |
| | | pCX117 | | | | | | |

Due to their superior induction and roles in numerous processes, PEA and Tyra were selected as targets for engineering selectivity in the TynA-FeaR system. This system presents an interesting opportunity to optimize ligand selectivity using two different knobs of control. Either TynA or FeaR can be engineered for selectivity. However, the protein structure and mode of ligand binding of FeaR have not been elucidated. In contrast, the structure of TynA has been characterized. Thus, improving the amine selectivity of TynA was focused on first. Several residues in TynA have been identified as catalytically essential, including D413 and Y496 (see e.g., FIG. 4A and FIG. 15). To optimize the selectivity of TynA to PEA and Tyra without fully eliminating enzymatic activity, the amino acids adjacent to these two essential residues were individually mutated, including positions 411-412, 414-416, 493-495, and 497-498.

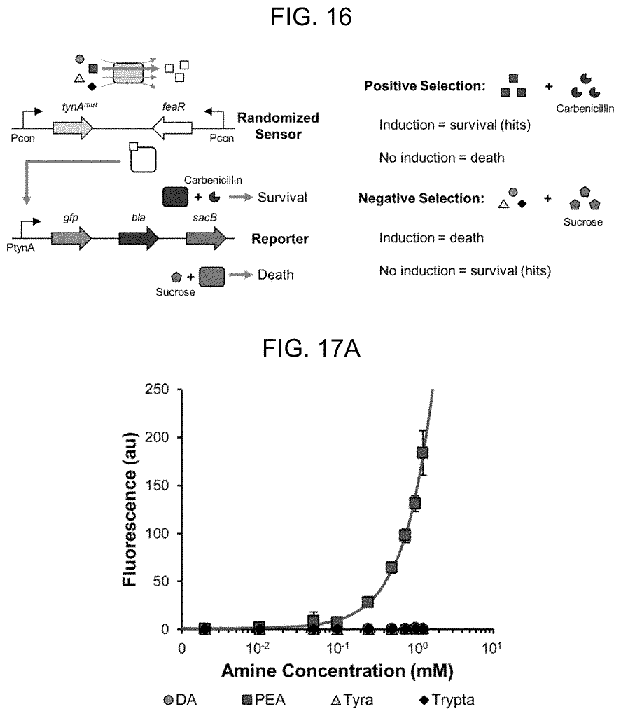

To quickly screen variants from the libraries for a response to each ligand, a growth-based assay was developed by incorporating the carbenicillin resistance gene, bla, and the sucrose counterselection gene, sacB, onto the reporter plasmid under the control of PtynA (see e.g., FIG. 16). When the constitutively expressed TynA produces an aldehyde, both Bla and SacB are expressed. To isolate variants with improved selectivity to a specific amine, rounds of positive selection (with the amine of interest and carbenicillin) and negative selection (with all other amines and sucrose) can be performed. In positive selection, cells that fail to respond to the amine of interest will be killed by carbenicillin. In negative selection, cells that remain promiscuous and respond to one of the undesired amines will be killed by sucrose. After multiple rounds of these dual selections, the activities of variants were validated by fluorescence measurements.

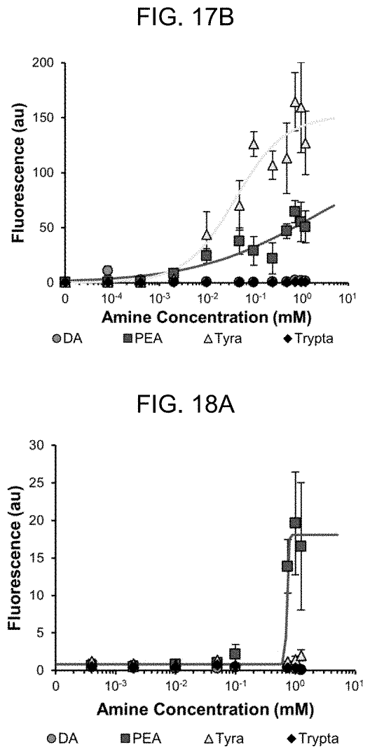

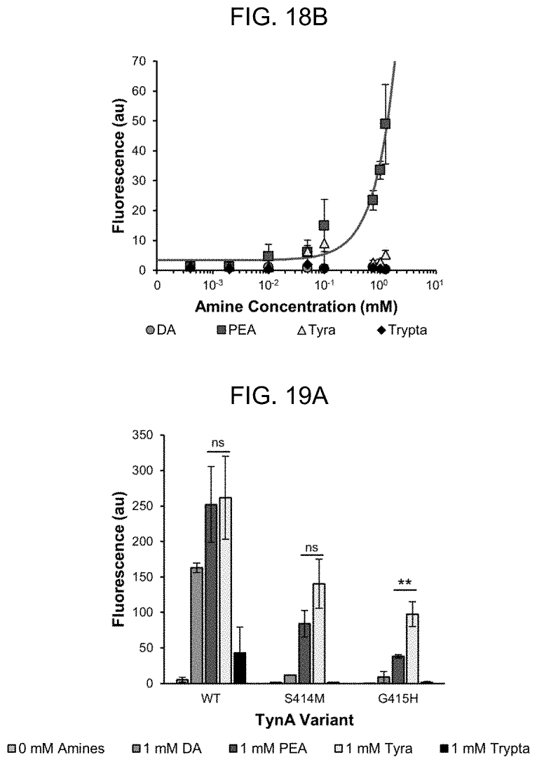

Two PEA-specific variants were successfully identified (see e.g., FIG. 4B). Both variants possessed a G494S mutation in TynA. However, one variant displayed a stronger fluorescence output in response to PEA. Upon further investigation, an A81T mutation in the FeaR protein of the stronger sensor was identified. Under the pressure of carbenicillin in the dual-selection system, a cell bearing the weak PEA-specific G494S TynA mutant additionally acquired the A81T FeaR mutation to improve b/a expression and cell survival. This double mutant sensor (G494S*) displayed 140-fold induction in response to PEA with an insignificant response to other tested amines (see e.g., FIG. 4C and FIG. 17A), while the single mutant sensor (G494S) displayed 50-fold induction by PEA (see e.g., FIG. 18A-FIG. 18B).

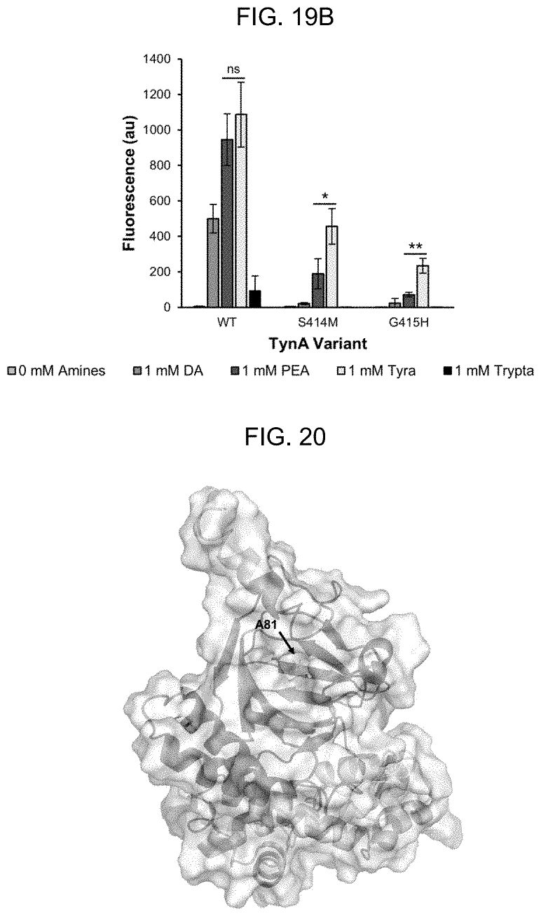

Two variants with improved selectivity to Tyra were also identified (see e.g., FIG. 19A-FIG. 19B). The best sensor, G415H, induced expression of the reporter by 200-fold and 79-fold in response to Tyra and PEA, respectively, with minimal response to DA and Trypta (see e.g., FIG. 4B, FIG. 4D, and FIG. 17B). The G494S and G494S* variants are the PEA-specific biosensors and the G415H variant is the Tyra-selective biosensor. In addition, this work further demonstrates how the ligand specificity of a protein can be quickly and effectively optimized through targeted mutagenesis of catalytically adjacent residues.

Characterization and Engineering of FeaR

Intrigued by the appearance of the A81T FeaR mutation in the G494S* sensor, next the importance of the 81st residue for ligand binding was explored. A protein motif search indicated that the position 12-185 of FeaR is a ligand-binding domain of the AraC-like TF, and the position 218-298 is its DNA-binding domain with a helix-turn-helix motif at positions 258-298. Since no experimentally derived structures of FeaR are known to exist, computational simulations were applied based on comparative modeling to predict the structure. The algorithm utilized the moderate homology of FeaR to CuxR, another AraC-like TF with a previously solved structure, to comparatively predict the structure of FeaR. Visualization of the predicted structure revealed that A81 is part of a solvent-accessible beta-barrel, and the alanine side chain is oriented toward the interior of the barrel (see e.g., FIG. 20). Based on this information, it was hypothesized that the beta-barrel is likely the aromatic aldehyde ligand-binding site. In agreement with this theory, the ligand docking simulations using wild-type FeaR found the beta-barrel to be the most thermodynamically favorable ligand-binding site within the protein, with A81 oriented in close proximity to the bound ligand (see e.g., FIG. 5A).

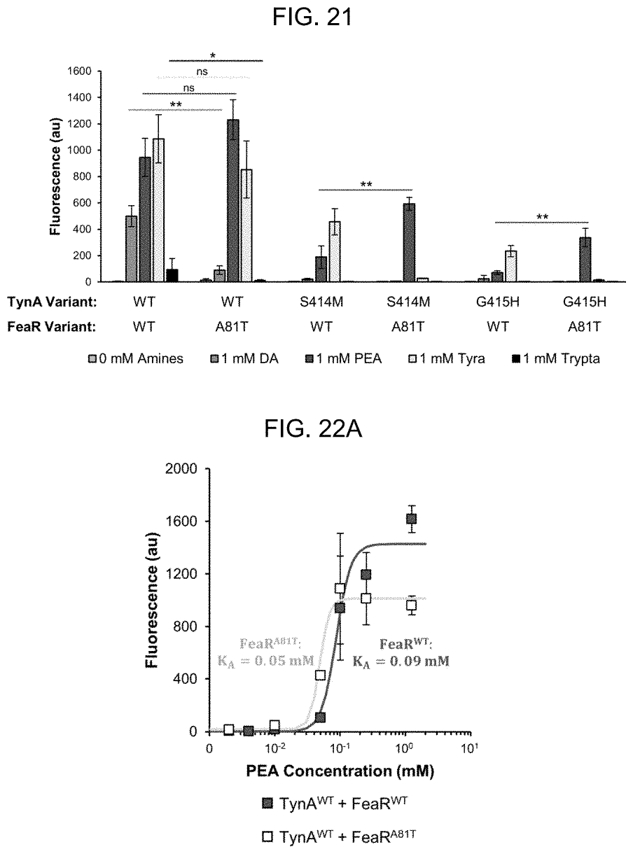

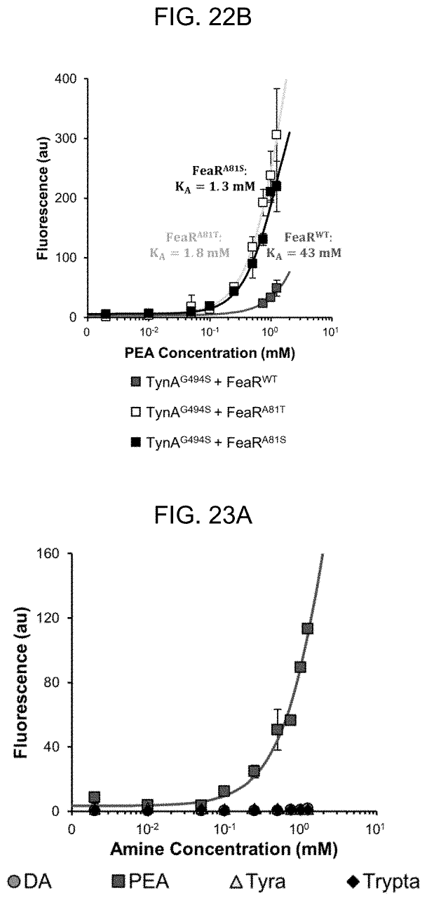

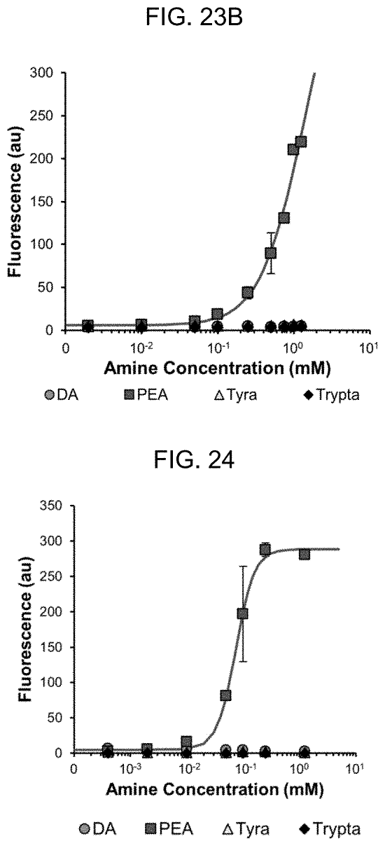

To further elucidate the role of the A81T mutation in ligand selectivity, the A81T mutation was inserted into the FeaR-TynA sensor plasmid with wild-type TynA. The mutation had an insignificant impact on the maximum response of the sensor to PEA and Tyra but significantly reduced the responses to DA and Trypta (see e.g., FIG. 21). However, when the same mutation was inserted into the sensor plasmids with the Tyra-selective S414M and G415H TynA variants, both sensors became PEA-selective (see e.g., FIG. 21). The fluorescence output of each variant in response to PEA was also improved by the A81T mutation. The improved PEA-dependent output and PEA selectivity created by the A81T mutation may be a result of an altered sensitivity profile, where A81T FeaR is more sensitive to PEA-aldehyde and less sensitive to the remaining aldehydes. While the S414M and G415H TynA mutations weaken the catalytic activity of the enzyme, unequally reducing the amount of each aldehyde produced (as shown by the lower fluorescence output of the two variants relative to wild-type TynA; see e.g., FIG. 19A-FIG. 19B), the A81T FeaR mutation may improve the sensitivity, and thereby selectivity, of FeaR to PEA-aldehyde. However, the high catalytic activity of wild-type TynA toward Tyra may be sufficient to overcome the elevated barrier of activation required by A81T FeaR to induce Tyra-dependent expression. Indeed, the A81T FeaR mutant demonstrated a lower K_{A} value than wild-type FeaR when paired with both wild-type and G494S TynA (see e.g., FIG. 22A-FIG. 22B). The A81S FeaR mutant paired with G494S TynA showed a very similar transfer function to that of A81T FeaR/G494S TynA, possibly due to the structural similarity of the side chains (see e.g., FIG. 22A-FIG. 22B and FIG. 23A-FIG. 23B).

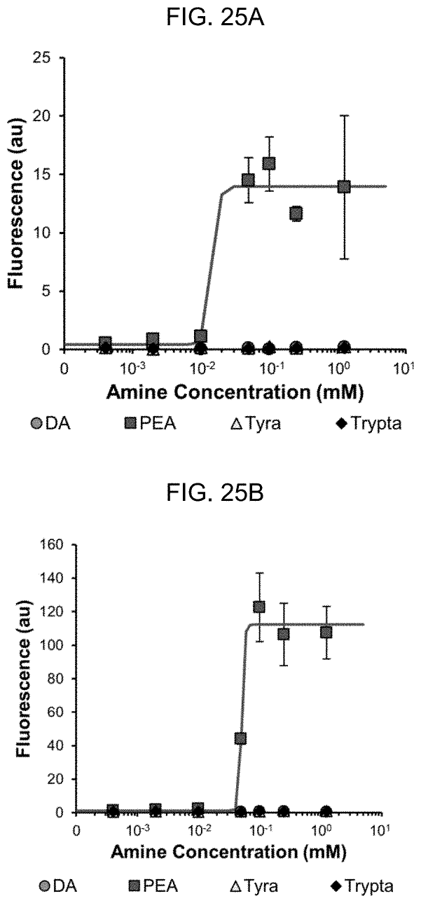

Noting the PEA selectivity given by the A81T mutation and the smallest size of the PEA-aldehyde (no OH group), followed by that of Tyra—(1 OH), DA—(2 OHs), and Trypta—(additional 5-membered ring) aldehyde (see e.g., FIG. 3A), it was hypothesized that small residues, including alanine as in wild-type FeaR, would cause ligand promiscuity, with ligand size-dependent response as shown in FIG. 3D. Slightly larger residues such as threonine would have improved selectivity toward the smallest PEA-aldehyde, but much larger or charged residues may fully eliminate ligand binding. It was also hypothesized that the hydropathy index of the 81st residue may also play a role in the sensor's performance due to differences in ligand polarities caused by the different number of hydroxyl groups. Notably, amines are positively charged in physiological pH, but the true ligands are uncharged aldehydes. To test this prediction, A81 was mutated and each mutant was paired with the promiscuous wild-type TynA (see e.g., FIG. 5B). Consistent with the predictions, A81L, A81P, A81 I, and A81N were identified as PEA-specific variants, with 580-fold induction for the A81L sensor (see e.g., FIG. 5C). The A81 L and A81P variants also demonstrated improved sensitivities to PEA relative to the G494S* PEA-specific sensor, with half-maximal expression at 0.14 and 0.05 mM PEA, respectively (see e.g., FIG. 5C, FIG. 24, and FIG. 25A-FIG. 25B, TABLE 2). As predicted a clear pattern between the identity of residue 81 and ligand selectivity was observed (see e.g., FIG. 5D, TABLE 3).

TABLE 3
Ligand specificity of FeaR with different residues in the 81
position. Residues are listed in order of increasing size.

| Amino acid | FeaR A81 | Size (×10⁻³ | | | |
|---|---|---|---|---|---|
| family | variant | nm³) | HPI | Phenotype | Selectivity |
| Hydrophobic | Glycine (G) | 59.9 | −0.4 | PEA/Tyra | PEA ≈ Tyra > |
| | | | | selective | DA |
| | Alanine (A) | 87.8 | 1.8 | Non specific | PEA ≈ Tyra > |
| | | | | | DA > Trypta |
| | Proline (P)* | 123.3 | −1.6 | PEA specific | PEA only |
| | Valine (V) | 138.8 | 4.2 | PEA/Tyra | PEA > Tyra |
| | | | | selective | No DA or |
| | | | | | Trypta |
| | | | | | activity |
| | Methionine (M) | 165.2 | 1.9 | PEA/Tyra | PEA ≈ Tyra > |
| | | | | selective | DA No |
| | | | | | Trypta |
| | | | | | activity |
| | Isoleucine (I) | 166.1 | 4.5 | PEA specific | PEA only |
| | Leucine (L) | 168 | 3.8 | PEA specific | PEA only |
| | Phenylalanine (F)** | 189.7 | 2.8 | No activity | No activity |
| | Tyrosine (Y)** | 191.2 | −1.3 | No activity | No activity |
| | Tryptophan (W)** | 227.9 | −0.9 | No activity | No activity |
| Polar | Serine (S) | 91.7 | −0.8 | PEA/Tyra | PEA > Tyra > |
| | | | | selective | DA > Trypta |
| | Cysteine (C) | 105.4 | 2.5 | Non specific | PEA > Tyra > |
| | | | | | DA > Trypta |
| | Threonine (T) | 118.3 | 0.7 | PEA/Tyra | PEA > Tyra > |
| | | | | selective | DA > Trypta |
| | Asparagine (N) | 120.1 | −3.5 | PEA specific | PEA only |
| | Glutamine (Q) | 145.1 | −3.5 | No activity | No activity |
| Charged | Histidine (H) | 156.3 | −3.2 | No activity | No activity |
| (positive) | | | | | |
| | Lysine (K) | 172.7 | −3.9 | No activity | No activity |
| | Arginine (R) | 188.2 | −4.5 | No activity | No activity |
| Charged | Aspartic Acid (D) | 115.4 | −3.5 | No activity | No activity |
| (negative) | | | | | |
| | Glutamic Acid (E) | 140.9 | −3.5 | No activity | No activity |

*P is more structurally rigid than the other amino acids.
**F, Y, and W are aromatic amino acids.
HPI, hydropathy index.

FeaR recognized Tyra-aldehyde at a range of size and hydropathy index values (see e.g., FIG. 5D; blue area). The ranges with Tyra-dependent activity were non-discrete (as shown by the shape of the blue area). This suggests that the size and polarity of the 81st residue collaboratively dictate ligand selectivity. Small deviations outside of this space allowed FeaR to maintain PEA-aldehyde activity, but not Tyra activity. Similarly, the ranges that allowed binding of FeaR to the larger DA-aldehyde and Trypta-aldehyde ligands were smaller. Large deviations in either residue size or hydropathy index caused a complete loss of activity in response to all ligands.

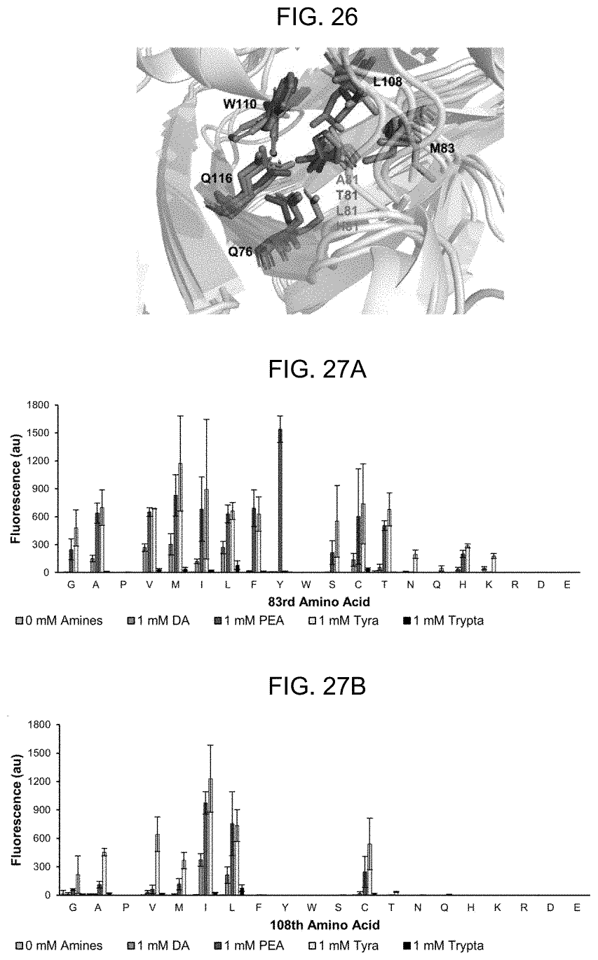

Structural simulations of the wild-type (promiscuous), A81T (Tyra- and PEA-responsive), A81L (PEA-specific), and A81H (non-functional) variants were performed to understand how the mutations may alter the structure and thus ligand binding of FeaR. Residues of increasing size (A<T<L) protruded further into and occupied more space in the ligand-binding pocket, which is consistent with the observed size effect. All three mutations also shifted the positions of several side chains within the beta-barrel relative to the wild-type structure (see e.g., FIG. 26), contributing to the contrasting ligand selectivity. Of particular interest are the differences in the three residues forming the top of the pocket, M83, L108, and W110, as each has severe differences in rotation angles. However, no consistent trends exist between the size of the 81st residue and the orientation of these positions.

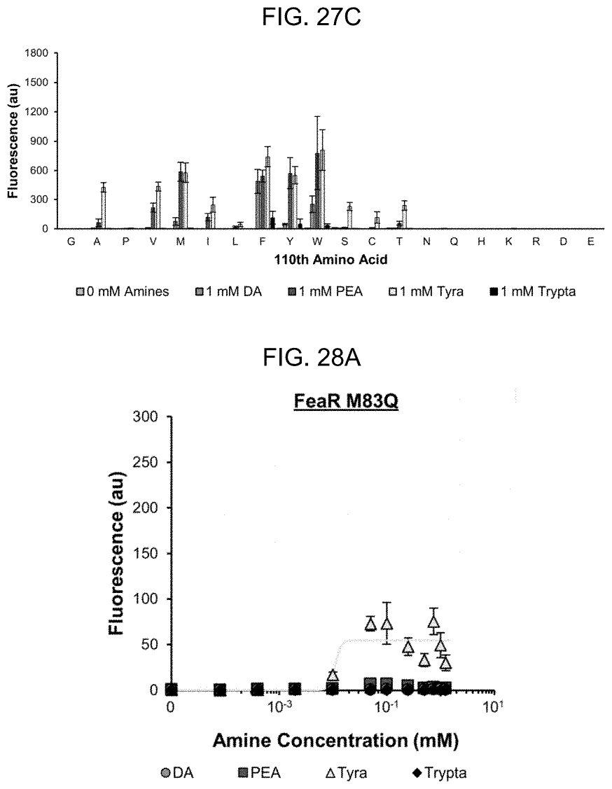

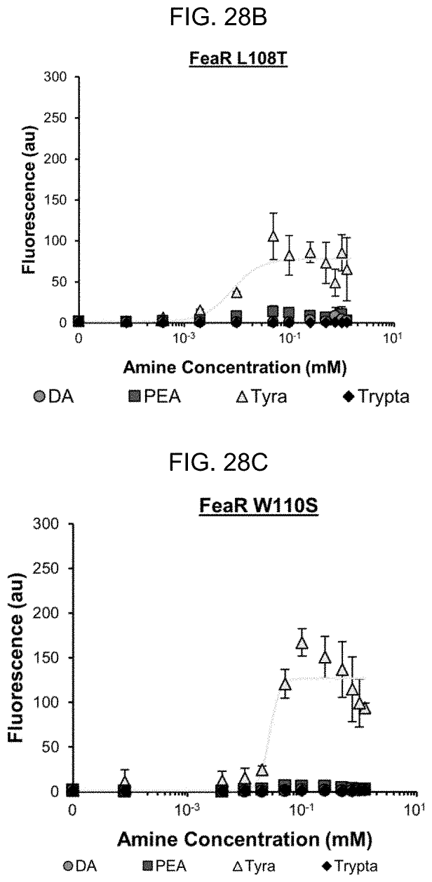

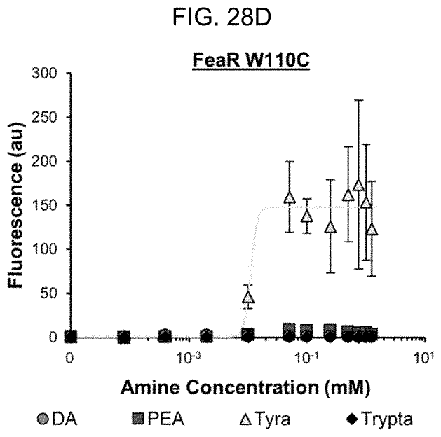

Given their significant rotations, it was hypothesized that residues M83, L108, and W110 may also play important roles in ligand binding, potentially by making contacts with alternative functional groups in the ligand. It was hypothesized that mutagenizing these residues may reveal a sensor for DA, Tyra, or Trypta. Each residue was individually mutated and multiple ligand-specific sensors for PEA or Tyra were identified (see e.g., FIG. 6A-6C and FIG. 27A-FIG. 27C). Randomizing the amino acids in positions 108 and 110 was only able to generate Tyra-specific variants, while randomizing the amino acid in position 83 was able to generate both Tyra- and PEA-specific variants. Each amino acid position demonstrated unique size and hydropathy index profiles for ligand specificity. These ligand-specific sensors were further characterized to assess their ligand sensitivity (see e.g., FIG. 6D, FIG. 6E, FIG. 28A-FIG. 28D). The M83Y FeaR variant displayed the most sensitive response of all PEA-specific sensors, with half-maximal expression at 0.012 mM PEA. The best Tyra-specific variant, M83N, recognized its ligand with a comparable sensitivity.

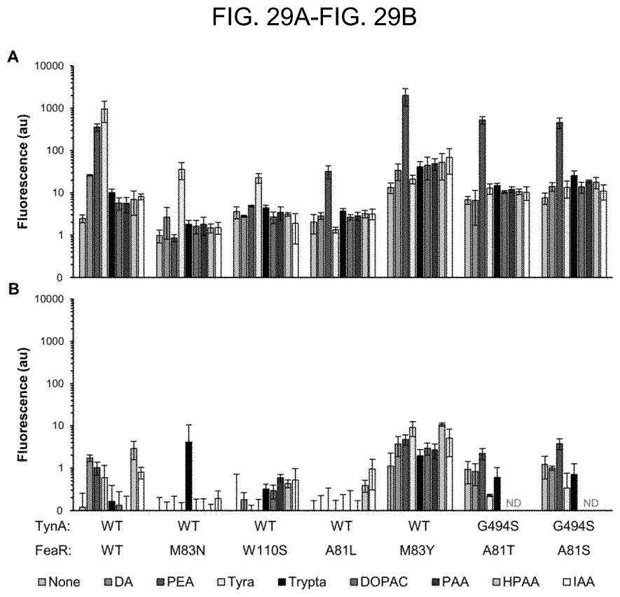

To further confirm the specificity of the best performing sensors, the response of each to the four amines and the four respective carboxylic acids, 3,4-dihydroxyphenylacetic acid (DOPAC), phenylacetic acid (PAA), 4-hydroxyphenylacetic acid (HPAA), and indole-3-acetic acid (IAA) was tested in minimal medium with casamino acids and LB medium (see e.g., FIG. 29A-FIG. 29B). No sensors significantly responded to the carboxylic acids in either medium, confirming the specificity of FeaR to only aldehydes. All mutant sensors remained specific to PEA or Tyra in minimal medium with casamino acids. However, all sensors displayed no output in LB medium, suggesting that the system may be regulated by catabolite repression.

Together, this work provides insights into the structure and activity of FeaR. In addition, by engineering FeaR, the best performing PEA- and Tyra-specific sensors are provided for future applications. Future work could include random approaches of protein engineering such as error-prone PCR-based directed evolution to develop additional ligand-specific sensors for the larger DA and Trypta amines. In addition, further exploration of the relevant regulatory pathways is required to uncouple the activity of the sensors with resource availability.

Discussion

The ligand-specific sensors developed here have the potential for diverse applications, including (1) monitoring food quality, (2) diagnosing or treating metabolic, digestive, and neurological disorders in probiotics or ex vivo wearable, paper-based and cell-free systems, and (3) dynamically regulating enzymatic pathways for microbial metabolic engineering. The high degree of ligand specificity shown by the engineered sensors allows them to effectively differentiate between diverse structurally similar aromatic metabolites. Demonstrated herein is an efficient and effective method of rational protein engineering by individually performing saturation mutagenesis on logically selected amino acid residues. The generalizability of this method is shown by applying it to three protein systems, including both enzymes and TFs. The simplicity of the approach and small library sizes make it an attractive first step in sensor engineering. Although this protein engineering method requires basic structural knowledge of the protein of interest, the scope of the required information is less than fully computation-based engineering approaches. However, also shown herein is how protein simulations can be used to identify important residues from uncharacterized proteins.

Protein engineering for specific ligand-protein interactions has been extensively performed, especially for facilitating chemical drug screening. However, coupling ligand-protein binding with an output response has remained challenging. Current approaches include designing proteins that fluoresce upon ligand binding or employing ligand-binding TFs. While the former can be used to develop sensitive sensors, the latter can generate sensors as well as controllers for downstream functions such as gene expression. Because this TF-based system requires the maintenance or engineering of DNA-protein interaction in addition to engineering ligand-protein binding, developing ligand-specific sensors using this system has been challenging, especially when the target ligands are structurally similar. To address this challenging issue, two approaches were demonstrated: optimizing ligand-TF binding specificity and sensitivity by leveraging differential multimerization patterns of TyrR without affecting DNA-TF binding interactions (see e.g., FIG. 1A-FIG. 1C and FIG. 2A-FIG. 2G) and employing the dual-knob modulation of the specificity and sensitivity of substrate-enzyme and ligand-TF interactions while maintaining DNA-TF-binding interactions (see e.g., FIG. 3A-FIG. 3D, FIG. 4A-FIG. 4D, see e.g., FIG. 5A-FIG. 5D, and FIG. 6A-FIG. 6E).

The feaR-tynA system represents a unique way to develop sensors using two different control knobs. As shown in FIG. 4A-FIG. 4D and FIG. 21, FIG. 22A-FIG. 22B, and FIG. 23A-FIG. 23B, Tyra-selective sensors (S414M or G415H TynA) can be transformed into PEA-specific sensors by introducing A81T FeaR, sensor sensitivity (G494S TynA) can be improved by A81T or A81S FeaR mutations, and PEA-Tyra-selective sensors (A81T or A81S FeaR) can become PEA-specific sensors when combined with G494S TynA. One caveat is that TynA and FeaR mutagenesis often led to reduced output signals, sometimes making a PEA-specific sensor (FeaR-A81P) not responsive to any ligand due to lowered aldehyde concentrations (e.g., TynA-G494S+FeaR-A81P). The output of the sensors could be further improved by optimization of the ribosome binding site of the reporter or mutagenesis of the PtynA reporter promoter. Furthermore, for engineered TynA variants, increasing the expression of the enzyme through optimization of its promoter or ribosome binding site may improve signal output by increasing the amine to aldehyde conversion rate. Notably, the intermediate aldehydes are rarely found in natural environments, eliminating the possibility of extracellular aldehydes contributing to sensor outputs. However, until the sensor activity is uncoupled from catabolite repression, applications may be limited to environments lacking a preferred carbon or nitrogen source.

Altogether, the specific microbial sensors for Phe, Tyr, PEA, and Tyra were generated. This work provides ligand-specific sensors that can be applied to create probiotics for diverse applications. In addition, the generalizable protein engineering techniques demonstrated here can be used to quickly and effectively engineer enzymes and TFs for ligand specificity and sensitivity. Although sensors were specifically developed with potential applications primarily in medicine and food quality, these enzymes and sensors can be similarly engineered to produce fuels, pharmaceuticals, and commodity chemicals in response to the levels of metabolites or the products in a dynamic way. This work represents a considerable achievement in the challenging goal of engineering ligand-specific sense-and-respond systems for medically relevant chemicals through coupling protein conformational changes caused by ligand binding with DNA interactions.

Methods

Plasmids, Strains, and Reagents

All plasmids, strains, and genetic parts used in this study are summarized in TABLE 4, TABLE 5, and TABLE 6, respectively.

It is noted that TrpR and Ptrp constructs are either WT sequences form E. coli or from the following paper: Ellefson, J. W., Ledbetter, M. P. & Ellington, A. D. Directed evolution of a synthetic phylogeny of programmable Trp repressors. Nat Chem Biol 14, 361-367 (2018). https://doi.org/10.1038/s41589-018-0006-7.

TABLE 4
Plasmids used in this work.

| Plasmid | Genetic | | Antibiotic | |
|---|---|---|---|---|
| Name | Parts | Origin | Resistance | Source |
| Recombineering | | | | |
| pMP11 | Pcon-cas9 + pBAD-λ | oriR101 | Ampicillin | Mehrer et al. |
| | Red genes + Ptet- | | | |
| | sgRNA-pBR322ori | | | |
| pgRNAcm | Constitutive sgRNA | pBR322 | Chloramphenicol | Mehrer et al. |
| pMBA016 | pgRNA-trpR | pBR322 | Chloramphenicol | This work |
| TrpR-based tryptophan, | | | | |
| 5-hydroxytryptophan, | | | | |
| and tryptamine sensors | | | | |
| pMBA014 | Ptrp-gfp | ColE1 | Chloramphenicol | This |
| | | | | work; |
| | | | | Ptrp from |
| | | | | Ellefson |
| | | | | et al. |
| pMBA035 | P17-trpR | p15A | Spectinomycin | This work |
| pMBA077 | Ptrp(O_{D})-gfp | ColE1 | Chloramphenicol | This work |
| pMBA094 | Ptrp(O₁)-gfp | ColE1 | Chloramphenicol | This work |
| pMBA103 | P17-trpR(O_{D}) | p15A | Spectinomycin | This work |
| pMBA173 | P17-trpR(O₁) | p15A | Spectinomycin | This work |
| TyrR-based | | | | |
| phenylalanine and | | | | |
| tyrosine sensors | | | | |
| pAGR001 | PtyrR-gfp | ColE1 | Chloramphenicol | This work |
| pAGR002 | Pmtr-gfp | ColE1 | Chloramphenicol | This work |
| pAGR003 | PtyrB-gfp | ColE1 | Chloramphenicol | This work |
| pAGR009 | ParoF-gfp | ColE1 | Chloramphenicol | This work |
| pAGR010 | PtyrP-gfp | ColE1 | Chloramphenicol | This work |
| pAGR011 | ParoP-gfp | ColE1 | Chloramphenicol | This work |
| pAGR023 | PtyrP-gfp + PtyrR-tyrR | ColE1 | Chloramphenicol | This |
| | | | | work; |
| | | | | derived |
| | | | | from |
| | | | | pAGR010 |
| pAGR031 | ParoF*-gfp + PtyrR-tyrR | ColE1 | Chloramphenicol | This |
| | | | | work; |
| | | | | derived |
| | | | | from |
| | | | | pAGR009 |
| pAGR032 | ParoP*-gfp + PtyrR-tyrR | ColE1 | Chloramphenicol | This |
| | | | | work; |
| | | | | derived |
| | | | | from |
| | | | | pAGR011 |
| pAGR102 | PtyrP-gfp + PtyrR- | ColE1 | Chloramphenicol | This |
| | tyrR_E274Q | | | work; |
| | | | | derived |
| | | | | from |
| | | | | pAGR023 |
| pAGR155 | PtyrP-gfp + PtyrR- | ColE1 | Chloramphenicol | This |
| | tyrR_E274Q_L11G | | | work; |
| | | | | derived |
| | | | | from |
| | | | | pAGR102 |
| pAGR156 | PtyrP-gfp + PtyrR- | ColE1 | Chloramphenicol | This |
| | tyrR_E274Q_T14G | | | work; |
| | | | | derived |
| | | | | from |
| | | | | pAGR102 |
| pAGR157 | PtyrP-gfp + PtyrR- | ColE1 | Chloramphenicol | This |
| | tyrR_E274Q_T14V | | | work; |
| | | | | derived |
| | | | | from |
| | | | | pAGR102 |
| pAGR158 | PtyrP-gfp + PtyrR- | ColE1 | Chloramphenicol | This |
| | tyrR_E274Q_D103H | | | work; |
| | | | | derived |
| | | | | from |
| | | | | pAGR102 |
| pAGR159 | PtyrP-gfp + PtyrR- | ColE1 | Chloramphenicol | This |
| | tyrR_E274Q_D103S | | | work; |
| | | | | derived |
| | | | | from |
| | | | | pAGR102 |
| pAGR160 | PtyrP-gfp + PtyrR- | ColE1 | Chloramphenicol | This |
| | tyrR_E274Q_C7V | | | work; |
| | | | | derived |
| | | | | from |
| | | | | pAGR102 |
| pAGR473 | PtyrP-gfp + PtyrR- | ColE1 | Chloramphenicol | This |
| | tyrR_R10F | | | work; |
| | | | | derived |
| | | | | from |
| | | | | pAGR023 |
| pAGR475 | PtyrP-gfp + PtyrR- | ColE1 | Chloramphenicol | This |
| | tyrR_L11G | | | work; |
| | | | | derived |
| | | | | from |
| | | | | pAGR023 |
| pAGR476 | PtyrP-gfp + PtyrR- | ColE1 | Chloramphenicol | This |
| | tyrR_G12T | | | work; |
| | | | | derived |
| | | | | from |
| | | | | pAGR023 |
| pAGR477 | PtyrP-gfp + PtyrR- | ColE1 | Chloramphenicol | This |
| | tyrR_L13G | | | work; |
| | | | | derived |
| | | | | from |
| | | | | pAGR023 |
| pAGR479 | PtyrP-gfp + PtyrR- | ColE1 | Chloramphenicol | This |
| | tyrR_T14K | | | work; |
| | | | | derived |
| | | | | from |
| | | | | pAGR023 |
| TynA-FeaR-based | | | | |
| amine sensors | | | | |
| pHJY022 | Pcon-feaR + Pcon-tynA | pSC101 | Kanamycin | This work |
| pHJY023 | Pcon-feaR | pSC101 | Kanamycin | This work |
| pHJY028 | PtynA-gfp | p15A | Spectinomycin | This work |
| pCX008 | PtynA-gfp-bla-sacB | p15A | Spectinomycin | This work |
| pCX010 | Pcon-feaR + Pcon- | pSC101 | Kanamycin | This |
| | tynA_G494S | | | work; |
| | | | | derived |
| | | | | from |
| | | | | pHJY022 |
| pCX014 | Pcon-feaR_A81T + | pSC101 | Kanamycin | This |
| | Pcon-tynA_G494S | | | work; |
| | | | | derived |
| | | | | from |
| | | | | pHJY022 |
| pCX015 | Pcon-feaR + Pcon- | pSC101 | Kanamycin | This |
| | tynA_G415H | | | work; |
| | | | | derived |
| | | | | from |
| | | | | pHJY022 |
| pCX019 | Pcon-feaR + Pcon- | pSC101 | Kanamycin | This |
| | tynA_S414M | | | work; |
| | | | | derived |
| | | | | from |
| | | | | pHJY022 |
| pCX020 | Pcon-feaR + Pcon- | pSC101 | Kanamycin | This |
| | tynA_G415Y | | | work; |
| | | | | derived |
| | | | | from |
| | | | | pHJY022 |
| pCX016 | Pcon-feaR_A81T + | pSC101 | Kanamycin | This |
| | Pcon-tynA | | | work; |
| | | | | derived |
| | | | | from |
| | | | | pHJY022 |
| pCX017 | Pcon-feaR_A81T + | pSC101 | Kanamycin | This |
| | Pcon-tynA_S414M | | | work; |
| | | | | derived |
| | | | | from |
| | | | | pCX019 |
| pCX018 | Pcon-feaR_A81T + | pSC101 | Kanamycin | This |
| | Pcon-tynA_G415H | | | work; |
| | | | | derived |
| | | | | from |
| | | | | pCX015 |
| pCX024 | Pcon-feaR_A81L + | pSC101 | Kanamycin | This |
| | Pcon-tynA | | | work; |
| | | | | derived |
| | | | | from |
| | | | | pHJY022 |
| pCX025 | Pcon-feaR_A81P + | pSC101 | Kanamycin | This |
| | Pcon-tynA | | | work; |
| | | | | derived |
| | | | | from |
| | | | | pHJY022 |
| pCX032 | Pcon-feaR_A81S + | pSC101 | Kanamycin | This |
| | Pcon-tynA_G494S | | | work; |
| | | | | derived |
| | | | | from |
| | | | | pCX010 |
| pCX033 | Pcon-feaR_A81S + | pSC101 | Kanamycin | This |
| | Pcon-tynA | | | work; |
| | | | | derived |
| | | | | from |
| | | | | pHJY022 |
| pCX035 | Pcon-feaR_A81V + | pSC101 | Kanamycin | This |
| | Pcon-tynA | | | work; |
| | | | | derived |
| | | | | from |
| | | | | pHJY022 |
| pCX036 | Pcon-feaR_A81E + | pSC101 | Kanamycin | This |
| | Pcon-tynA | | | work; |
| | | | | derived |
| | | | | from |
| | | | | pHJY022 |
| pCX037 | Pcon-feaR_A81M + | pSC101 | Kanamycin | This |
| | Pcon-tynA | | | work; |
| | | | | derived |
| | | | | from |
| | | | | pHJY022 |
| pCX039 | Pcon-feaR_A81G + | pSC101 | Kanamycin | This |
| | Pcon-tynA | | | work; |
| | | | | derived |
| | | | | from |
| | | | | pHJY022 |
| pCX040 | Pcon-feaR_A81I + | pSC101 | Kanamycin | This |
| | Pcon-tynA | | | work; |
| | | | | derived |
| | | | | from |
| | | | | pHJY022 |
| pCX041 | Pcon-feaR_A81F + | pSC101 | Kanamycin | This |
| | Pcon-tynA | | | work; |
| | | | | derived |
| | | | | from |
| | | | | pHJY022 |
| pCX042 | Pcon-feaR_A81Y + | pSC101 | Kanamycin | This |
| | Pcon-tynA | | | work; |
| | | | | derived |
| | | | | from |
| | | | | pHJY022 |
| pCX043 | Pcon-feaR_A81W + | pSC101 | Kanamycin | This |
| | Pcon-tynA | | | work; |
| | | | | derived |
| | | | | from |
| | | | | pHJY022 |
| pCX034 | Pcon-feaR_A81C + | pSC101 | Kanamycin | This |
| | Pcon-tynA | | | work; |
| | | | | derived |
| | | | | from |
| | | | | pHJY022 |
| pCX044 | Pcon-feaR_A81N + | pSC101 | Kanamycin | This |
| | Pcon-tynA | | | work; |
| | | | | derived |
| | | | | from |
| | | | | pHJY022 |
| pCX045 | Pcon-feaR_A81Q + | pSC101 | Kanamycin | This |
| | Pcon-tynA | | | work; |
| | | | | derived |
| | | | | from |
| | | | | pHJY022 |
| pCX046 | Pcon-feaR_A81K + | pSC101 | Kanamycin | This |
| | Pcon-tynA | | | work; |
| | | | | derived |
| | | | | from |
| | | | | pHJY022 |
| pCX047 | Pcon-feaR_A81H + | pSC101 | Kanamycin | This |
| | Pcon-tynA | | | work; |
| | | | | derived |
| | | | | from |
| | | | | pHJY022 |
| pCX048 | Pcon-feaR_A81R + | pSC101 | Kanamycin | This |
| | Pcon-tynA | | | work; |
| | | | | derived |
| | | | | from |
| | | | | pHJY022 |
| pCX049 | Pcon-feaR_A81D + | pSC101 | Kanamycin | This |
| | Pcon-tynA | | | work; |
| | | | | derived |
| | | | | from |
| | | | | pHJY022 |
| pCX075 | Pcon-feaR_M83G + | pSC101 | Kanamycin | This |
| | Pcon-tynA | | | work; |
| | | | | derived |
| | | | | from |
| | | | | pHJY022 |
| pCX072 | Pcon-feaR_M83A + | pSC101 | Kanamycin | This |
| | Pcon-tynA | | | work; |
| | | | | derived |
| | | | | from |
| | | | | pHJY022 |
| pCX070 | Pcon-feaR_M83P + | pSC101 | Kanamycin | This |
| | Pcon-tynA | | | work; |
| | | | | derived |
| | | | | from |
| | | | | pHJY022 |
| pCX076 | Pcon-feaR_M83V + | pSC101 | Kanamycin | This |
| | Pcon-tynA | | | work; |
| | | | | derived |
| | | | | from |
| | | | | pHJY022 |
| pCX077 | Pcon-feaR_M83I + | pSC101 | Kanamycin | This |
| | Pcon-tynA | | | work; |
| | | | | derived |
| | | | | from |
| | | | | pHJY022 |
| pCX078 | Pcon-feaR_M83L + | pSC101 | Kanamycin | This |
| | Pcon-tynA | | | work; |
| | | | | derived |
| | | | | from |
| | | | | pHJY022 |
| pCX074 | Pcon-feaR_M83F + | pSC101 | Kanamycin | This |
| | Pcon-tynA | | | work; |
| | | | | derived |
| | | | | from |
| | | | | pHJY022 |
| pCX073 | Pcon-feaR_M83Y + | pSC101 | Kanamycin | This |
| | Pcon-tynA | | | work; |
| | | | | derived |
| | | | | from |
| | | | | pHJY022 |
| pCX080 | Pcon-feaR_M83W + | pSC101 | Kanamycin | This |
| | Pcon-tynA | | | work; |
| | | | | derived |
| | | | | from |
| | | | | pHJY022 |
| pCX081 | Pcon-feaR_M83S + | pSC101 | Kanamycin | This |
| | Pcon-tynA | | | work; |
| | | | | derived |
| | | | | from |
| | | | | pHJY022 |
| pCX082 | Pcon-feaR_M83C + | pSC101 | Kanamycin | This |
| | Pcon-tynA | | | work; |
| | | | | derived |
| | | | | from |
| | | | | pHJY022 |
| pCX071 | Pcon-feaR_M83T + | pSC101 | Kanamycin | This |
| | Pcon-tynA | | | work; |
| | | | | derived |
| | | | | from |
| | | | | pHJY022 |
| pCX068 | Pcon-feaR_M83N + | pSC101 | Kanamycin | This |
| | Pcon-tynA | | | work; |
| | | | | derived |
| | | | | from |
| | | | | pHJY022 |
| pCX083 | Pcon-feaR_M83Q + | pSC101 | Kanamycin | This |
| | Pcon-tynA | | | work; |
| | | | | derived |
| | | | | from |
| | | | | pHJY022 |
| pCX084 | Pcon-feaR_M83H + | pSC101 | Kanamycin | This |
| | Pcon-tynA | | | work; |
| | | | | derived |
| | | | | from |
| | | | | pHJY022 |
| pCX085 | Pcon-feaR_M83K + | pSC101 | Kanamycin | This |
| | Pcon-tynA | | | work; |
| | | | | derived |
| | | | | from |
| | | | | pHJY022 |
| pCX086 | Pcon-feaR_M83R + | pSC101 | Kanamycin | This |
| | Pcon-tynA | | | work; |
| | | | | derived |
| | | | | from |
| | | | | pHJY022 |
| pCX087 | Pcon-feaR_M83D + | pSC101 | Kanamycin | This |
| | Pcon-tynA | | | work; |
| | | | | derived |
| | | | | from |
| | | | | pHJY022 |
| pCX069 | Pcon-feaR_M83E + | pSC101 | Kanamycin | This |
| | Pcon-tynA | | | work; |
| | | | | derived |
| | | | | from |
| | | | | pHJY022 |
| pCX088 | Pcon-feaR_L108G + | pSC101 | Kanamycin | This |
| | Pcon-tynA | | | work; |
| | | | | derived |
| | | | | from |
| | | | | pHJY022 |
| pCX089 | Pcon-feaR_L108A + | pSC101 | Kanamycin | This |
| | Pcon-tynA | | | work; |
| | | | | derived |
| | | | | from |
| | | | | pHJY022 |
| pCX090 | Pcon-feaR_L108P + | pSC101 | Kanamycin | This |
| | Pcon-tynA | | | work; |
| | | | | derived |
| | | | | from |
| | | | | pHJY022 |
| pCX091 | Pcon-feaR_L108V + | pSC101 | Kanamycin | This |
| | Pcon-tynA | | | work; |
| | | | | derived |
| | | | | from |
| | | | | pHJY022 |
| pCX092 | Pcon-feaR_L108M + | pSC101 | Kanamycin | This |
| | Pcon-tynA | | | work; |
| | | | | derived |
| | | | | from |
| | | | | pHJY022 |
| pCX093 | Pcon-feaR_L108I + | pSC101 | Kanamycin | This |
| | Pcon-tynA | | | work; |
| | | | | derived |
| | | | | from |
| | | | | pHJY022 |
| pCX094 | Pcon-feaR_L108F + | pSC101 | Kanamycin | This |
| | Pcon-tynA | | | work; |
| | | | | derived |
| | | | | from |
| | | | | pHJY022 |
| pCX095 | Pcon-feaR_L108Y + | pSC101 | Kanamycin | This |
| | Pcon-tynA | | | work; |
| | | | | derived |
| | | | | from |
| | | | | pHJY022 |
| pCX096 | Pcon-feaR_L108W + | pSC101 | Kanamycin | This |
| | Pcon-tynA | | | work; |
| | | | | derived |
| | | | | from |
| | | | | pHJY022 |
| pCX097 | Pcon-feaR_L108S + | pSC101 | Kanamycin | This |
| | Pcon-tynA | | | work; |
| | | | | derived |
| | | | | from |
| | | | | pHJY022 |
| pCX098 | Pcon-feaR_L108C + | pSC101 | Kanamycin | This |
| | Pcon-tynA | | | work; |
| | | | | derived |
| | | | | from |
| | | | | pHJY022 |
| pCX099 | Pcon-feaR_L108T + | pSC101 | Kanamycin | This |
| | Pcon-tynA | | | work; |
| | | | | derived |
| | | | | from |
| | | | | pHJY022 |
| pCX100 | Pcon-feaR_L108N + | pSC101 | Kanamycin | This |
| | Pcon-tynA | | | work; |
| | | | | derived |
| | | | | from |
| | | | | pHJY022 |
| pCX101 | Pcon-feaR_L108Q + | pSC101 | Kanamycin | This |
| | Pcon-tynA | | | work; |
| | | | | derived |
| | | | | from |
| | | | | pHJY022 |
| pCX102 | Pcon-feaR_L108H + | pSC101 | Kanamycin | This |
| | Pcon-tynA | | | work; |
| | | | | derived |
| | | | | from |
| | | | | pHJY022 |
| pCX103 | Pcon-feaR_L108K + | pSC101 | Kanamycin | This |
| | Pcon-tynA | | | work; |
| | | | | derived |
| | | | | from |
| | | | | pHJY022 |
| pCX104 | Pcon-feaR_L108R + | pSC101 | Kanamycin | This |
| | Pcon-tynA | | | work; |
| | | | | derived |
| | | | | from |
| | | | | pHJY022 |
| pCX105 | Pcon-feaR_L108D + | pSC101 | Kanamycin | This |
| | Pcon-tynA | | | work; |
| | | | | derived |
| | | | | from |
| | | | | pHJY022 |
| pCX106 | Pcon-feaR_L108E + | pSC101 | Kanamycin | This |
| | Pcon-tynA | | | work; |
| | | | | derived |
| | | | | from |
| | | | | pHJY022 |
| pCX107 | Pcon-feaR_W110G + | pSC101 | Kanamycin | This |
| | Pcon-tynA | | | work; |
| | | | | derived |
| | | | | from |
| | | | | pHJY022 |
| pCX108 | Pcon-feaR_W110A + | pSC101 | Kanamycin | This |
| | Pcon-tynA | | | work; |
| | | | | derived |
| | | | | from |
| | | | | pHJY022 |
| pCX109 | Pcon-feaR_W110P + | pSC101 | Kanamycin | This |
| | Pcon-tynA | | | work; |
| | | | | derived |
| | | | | from |
| | | | | pHJY022 |
| pCX110 | Pcon-feaR_W110V + | pSC101 | Kanamycin | This |
| | Pcon-tynA | | | work; |
| | | | | derived |
| | | | | from |
| | | | | pHJY022 |
| pCX111 | Pcon-feaR_W110M + | pSC101 | Kanamycin | This |
| | Pcon-tynA | | | work; |
| | | | | derived |
| | | | | from |
| | | | | pHJY022 |
| pCX112 | Pcon-feaR_W110I + | pSC101 | Kanamycin | This |
| | Pcon-tynA | | | work; |
| | | | | derived |
| | | | | from |
| | | | | pHJY022 |
| pCX113 | Pcon-feaR_W110L + | pSC101 | Kanamycin | This |
| | Pcon-tynA | | | work; |
| | | | | derived |
| | | | | from |
| | | | | pHJY022 |
| pCX114 | Pcon-feaR_W110F + | pSC101 | Kanamycin | This |
| | Pcon-tynA | | | work; |
| | | | | derived |
| | | | | from |
| | | | | pHJY022 |
| pCX115 | Pcon-feaR_W110Y + | pSC101 | Kanamycin | This |
| | Pcon-tynA | | | work; |
| | | | | derived |
| | | | | from |
| | | | | pHJY022 |
| pCX116 | Pcon-feaR_W110S + | pSC101 | Kanamycin | This |
| | Pcon-tynA | | | work; |
| | | | | derived |
| | | | | from |
| | | | | pHJY022 |
| pCX117 | Pcon-feaR_W110C + | pSC101 | Kanamycin | This |
| | Pcon-tynA | | | work; |
| | | | | derived |
| | | | | from |
| | | | | pHJY022 |
| pCX118 | Pcon-feaR_W110T + | pSC101 | Kanamycin | This |
| | Pcon-tynA | | | work; |
| | | | | derived |
| | | | | from |
| | | | | pHJY022 |
| pCX119 | Pcon-feaR_W110N + | pSC101 | Kanamycin | This |
| | Pcon-tynA | | | work; |
| | | | | derived |
| | | | | from |
| | | | | pHJY022 |
| pCX120 | Pcon-feaR_W110Q + | pSC101 | Kanamycin | This |
| | Pcon-tynA | | | work; |
| | | | | derived |
| | | | | from |
| | | | | pHJY022 |
| pCX121 | Pcon-feaR_W110H + | pSC101 | Kanamycin | This |
| | Pcon-tynA | | | work; |
| | | | | derived |
| | | | | from |
| | | | | pHJY022 |
| pCX122 | Pcon-feaR_W110K+ | pSC101 | Kanamycin | This |
| | Pcon-tynA | | | work; |
| | | | | derived |
| | | | | from |
| | | | | pHJY022 |
| pCX123 | Pcon-feaR_W110R + | pSC101 | Kanamycin | This |
| | Pcon-tynA | | | work; |
| | | | | derived |
| | | | | from |
| | | | | pHJY022 |
| pCX124 | Pcon-feaR_W110D + | pSC101 | Kanamycin | This |
| | Pcon-tynA | | | work; |
| | | | | derived |
| | | | | from |
| | | | | pHJY022 |
| pCX125 | Pcon-feaR_W110E + | pSC101 | Kanamycin | This |
| | Pcon-tynA | | | work; |
| | | | | derived |
| | | | | from |
| | | | | pHJY022 |

TABLE 5
E. coli strains used in this work.

| Name | Strain | Source |
|---|---|---|
| sAGR004 | DH10B | Invitrogen |
| sAGR039 | Nissle 1917 (plasmid | DSMZ |
| | free) | |

TABLE 6
Genetic parts used in this work.

| SEQ ID | | |
|---|---|---|
| NO: | Part | Nucleotide Sequence |
| | General Parts | |
| 1 | gfpmut3 with RBS | aagtcggtgacagataacaggagtaagtaATGagtaaaggaga |
| | | agaacttttcactggagttgtcccaattcttgttgaattagatggtgatgtt |
| | | aatgggcacaaattttctgtcagtggagagggtgaaggtgatgcaac |
| | | atacggaaaacttacccttaaatttatttgcactactggaaaactacct |
| | | gttccatggccaacacttgtcactactttgacttatggtgttcaatgcttttc |
| | | aagatacccagatcatatgaaacggcatgactttttcaagagtgccat |
| | | gcccgaaggttatgtacaggaaagaactatatttttcaaagatgacg |
| | | ggaactataagacacgtgctgaagtcaagtttgaaggtgatacactt |
| | | gttaatagaatcgagttaaaaggtattgattttaaagaagatggaaac |
| | | attcttggacacaagttggaatacaactataactcacacaatgtatac |
| | | atcatggcagacaaacaaaagaatggaatcaaagttaacttcaaa |
| | | attagacacaacattgaagatggaagcgttcaactagcagaccatta |
| | | tcaacaaaatactccaattggcgatggccctgtccttttaccagacaa |
| | | ccattacctgtccacacaatctgccctttcgaaagatcccaacgaaa |
| | | agagagaccacatggtccttcttgagtttgtaacagctgctgggattac |
| | | acatggcatggatgaactatacaaaaggcctgcagcaaacgacga |
| | | aaactacgcttaagtagcttaa |
| 2 | BBa_J23117 (P17) | ttgacagctagctcagtcctagggattgtgctagc |
| | TyrR Sensor | |
| 3 | PtyrR | ccagcgaaaaataatgcaatatcgggtgctgaccggatatctttacg |
| | | ccgaagtgcccgtttttccgtctttgtgtcaatgattgttgacagaaacct |
| | | tcctgctatccaaatagtgtcatatcatcatatta |
| 4 | Pmtr | cgtcgtttcggtggtgatgcgtaatcatcgctgaacagcgaacacaat |
| | | ctgtaaaataatatatacagccccgatttttaccatcggggctttttttctg |
| | | tcttttgtactcgtgtactggtacagtgcaatgc |
| 5 | PtyrB with start | tctgttgctaattgccgttcgctcctgaacatccactcgatcttcgccttctt |
| | codon | ccggtttattgtgttttaaccacctgcccgtaaacctggagaaccatcg |
| | | cGTGtttcaaaaagttgacgcc |
| 6 | ParoF | attgttttcaaagggagtgtaaatttatctatacagaggtaagggttgaa |
| | | agcgcgactaaattgcctgtgtaaataaaaatgtacgaaatatggatt |
| | | gaaaactttactttatgtgttatcgttacgtc |
| 7 | PtyrP | cgtccctcctcaaaaaaagcctagcgtagcgattgccgcttatgaag |
| | | actttgcgccagcgcaggactgaatgctttttattgtacatttatatttaca |
| | | ccatatgtaacgtcggtttgacgaagcagccgttatgccttaacctgcg |
| | | ccgcag |
| 8 | ParoP | aagcaactcatcttcaaccatgcataaagcgggtgcattcgctgccg |
| | | cataccattattcttgatctgacggaagtctttttgtaacaattcaaacttc |
| | | tttgatgtaaacaaattaatacaacaaacggaattgcaaacttacaca |
| | | cgc |
| 9 | ParoF* | attgttttcaaagggagtgtaaatttatctatacagaggtaagggttgaa |
| | | agcgcgactaaattgcctgtgtaaataaaaatgtacgaaatatggatt |
| | | gaaaactttactttatgaggtataatgctagc |
| 10 | ParoP* | aagcaactcatcttcaaccatgcataaagcgggtgcattcgctgccg |
| | | cataccattattcttgacagctagctcagtcctaggtataatgctagcttc |
| | | tttgatgtaaacaaattaatacaacaaacggaattgcaaacttacaca |
| | | cgc |
| 11 | tyrR [with RBS] | [attgttcttttttcaggtgaaggttccc]ATGcgtctggaagtcttttgtg |
| | | aagaccgactcggtctgacccgcgaattactcgatctactcgtgctaa |
| | | gaggcattgatttacgcggtattgagattgatcccattgggcgaatcta |
| | | cctcaattttgctgaactggagtttgagagtttcagcagtctgatggccg |
| | | aaatacgccgtattgcgggtgttaccgatgtgcgtactgtcccgtggat |
| | | gccttccgaacgtgagcatctggcgttgagcgcgttactggaggcgtt |
| | | gcctgaacctgtgctctctgtcgatatgaaaagcaaagtggatatggc |
| | | gaacccggcgagctgtcagctttttgggcaaaaattggatcgcctgc |
| | | gcaaccataccgccgcacaattgattaacggctttaattttttacgttgg |
| | | ctggaaagcgaaccgcaagattcgcataacgagcatgtcgttattaa |
| | | tgggcagaatttcctgatggagattacgcctgtttatcttcaggatgaaa |
| | | atgatcaacacgtcctgaccggtgcggtggtgatgttgcgatcaacg |
| | | attcgtatgggccgccagttgcaaaatgtcgccgcccaggacgtcag |
| | | cgccttcagtcaaattgtcgccgtcagcccgaaaatgaagcatgttgt |
| | | cgaacaggcgcagaaactggcgatgctaagcgcgccgctgctgatt |
| | | acgggtgacacaggtacaggtaaagatctctttgcctacgcctgccat |
| | | caggcaagccccagagcgggcaaaccttacctggcgctgaactgt |
| | | gcgtctataccggaagatgcggtcgagagtgaactgtttggtcatgct |
| | | ccggaagggaagaaaggattctttgagcaggcgaacggtggttcg |
| | | gtgctgttggatgaaataggggaaatgtcaccacggatgcaggcga |
| | | aattactgcgtttccttaatgatggcactttccgtcgggttggcgaagac |
| | | catgaggtgcatgtcgatgtgcgggtgatttgcgctacgcagaagaat |
| | | ctggtcgaactggtgcaaaaaggcatgttccgtgaagatctctattatc |
| | | gtctgaacgtgttgacgctcaatctgccgccgctacgtgactgtccgc |
| | | aggacatcatgccgttaactgagctgttcgtcgcccgctttgccgacg |
| | | agcagggcgtgccgcgtccgaaactggccgctgacctgaatactgt |
| | | acttacgcgttatgcgtggccgggaaatgtgcggcagttaaagaacg |
| | | ctatctatcgcgcactgacacaactggacggttatgagctgcgtccac |
| | | aggatattttgttgccggattatgacgccgcaacggtagccgtgggcg |
| | | aagatgcgatggaaggttcgctggacgaaatcaccagccgttttgaa |
| | | cgctcggtattaacccagctttatcgcaattatcccagcacgcgcaaa |
| | | ctggcaaaacgtctcggcgtttcacataccgcgattgccaataagttg |
| | | cgggaatatggtctgagtcagaagaagaacgaagagtaa |
| 12 | tyrR_E274Q | ATGcgtctggaagtcttttgtgaagaccgactcggtctgacccgcga |
| | | attactcgatctactcgtgctaagaggcattgatttacgcggtattgaga |
| | | ttgatcccattgggcgaatctacctcaattttgctgaactggagtttgag |
| | | agtttcagcagtctgatggccgaaatacgccgtattgcgggtgttacc |
| | | gatgtgcgtactgtcccgtggatgccttccgaacgtgagcatctggcgt |
| | | tgagcgcgttactggaggcgttgcctgaacctgtgctctctgtcgatat |
| | | gaaaagcaaagtggatatggcgaacccggcgagctgtcagctttttg |
| | | ggcaaaaattggatcgcctgcgcaaccataccgccgcacaattgatt |
| | | aacggctttaattttttacgttggctggaaagcgaaccgcaagattcgc |
| | | ataacgagcatgtcgttattaatgggcagaatttcctgatggagattac |
| | | gcctgtttatcttcaggatgaaaatgatcaacacgtcctgaccggtgc |
| | | ggtggtgatgttgcgatcaacgattcgtatgggccgccagttgcaaaa |
| | | tgtcgccgcccaggacgtcagcgccttcagtcaaattgtcgccgtca |
| | | gcccgaaaatgaagcatgttgtcgaacaggcgcagaaactggcga |
| | | tgctaagcgcgccgctgctgattacgggtgacacaggtacaggtaa |
| | | agatctctttgcctacgcctgccatcaggcaagccccagagcgggc |
| | | aaaccttacctggcgctgaactgtgcgtctataccggaagatgcggtc |
| | | gagagtcaactgtttggtcatgctccggaagggaagaaaggattcttt |
| | | gagcaggcgaacggtggttcggtgctgttggatgaaataggggaaa |
| | | tgtcaccacggatgcaggcgaaattactgcgtttccttaatgatggca |
| | | ctttccgtcgggttggcgaagaccatgaggtgcatgtcgatgtgcggg |
| | | tgatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| 13 | tyrR_E274Q_L11G | ATGcgtctggaagtcttttgtgaagaccgagggggtctgacccgcg |
| | | aattactcgatctactcgtgctaagaggcattgatttacgcggtattgag |
| | | attgatcccattgggcgaatctacctcaattttgctgaactggagtttga |
| | | gagtttcagcagtctgatggccgaaatacgccgtattgcgggtgttac |
| | | cgatgtgcgtactgtcccgtggatgccttccgaacgtgagcatctggc |
| | | gttgagcgcgttactggaggcgttgcctgaacctgtgctctctgtcgat |
| | | atgaaaagcaaagtggatatggcgaacccggcgagctgtcagctttt |
| | | tgggcaaaaattggatcgcctgcgcaaccataccgccgcacaattg |
| | | attaacggctttaattttttacgttggctggaaagcgaaccgcaagattc |
| | | gcataacgagcatgtcgttattaatgggcagaatttcctgatggagatt |
| | | acgcctgtttatcttcaggatgaaaatgatcaacacgtcctgaccggt |
| | | gcggtggtgatgttgcgatcaacgattcgtatgggccgccagttgcaa |
| | | aatgtcgccgcccaggacgtcagcgccttcagtcaaattgtcgccgt |
| | | cagcccgaaaatgaagcatgttgtcgaacaggcgcagaaactggc |
| | | gatgctaagcgcgccgctgctgattacgggtgacacaggtacaggt |
| | | aaagatctctttgcctacgcctgccatcaggcaagccccagagcgg |
| | | gcaaaccttacctggcgctgaactgtgcgtctataccggaagatgcg |
| | | gtcgagagtcaactgtttggtcatgctccggaagggaagaaaggatt |
| | | ctttgagcaggcgaacggtggttcggtgctgttggatgaaatagggg |
| | | aaatgtcaccacggatgcaggcgaaattactgcgtttccttaatgatg |
| | | gcactttccgtcgggttggcgaagaccatgaggtgcatgtcgatgtgc |
| | | gggtgatttgcgctacgcagaagaatctggtcgaactggtgcaaaaa |
| | | ggcatgttccgtgaagatctctattatcgtctgaacgtgttgacgctcaa |
| | | tctgccgccgctacgtgactgtccgcaggacatcatgccgttaactga |
| | | gctgttcgtcgcccgctttgccgacgagcagggcgtgccgcgtccga |
| | | aactggccgctgacctgaatactgtacttacgcgttatgcgtggccgg |
| | | gaaatgtgcggcagttaaagaacgctatctatcgcgcactgacaca |
| | | actggacggttatgagctgcgtccacaggatattttgttgccggattatg |
| | | acgccgcaacggtagccgtgggcgaagatgcgatggaaggttcgc |
| | | tggacgaaatcaccagccgttttgaacgctcggtattaacccagcttta |
| | | tcgcaattatcccagcacgcgcaaactggcaaaacgtctcggcgttt |
| | | cacataccgcgattgccaataagttgcgggaatatggtctgagtcag |
| | | aagaagaacgaagagtaa |
| 14 | tyrR_E274Q_T14G | ATGcgtctggaagtcttttgtgaagaccgactcggtctgacccgcga |
| | | attactcgatctactcgtgctaagaggcattgatttacgcggtattgaga |
| | | ttgatcccattgggcgaatctacctcaattttgctgaactggagtttgag |
| | | agtttcagcagtctgatggccgaaatacgccgtattgcgggtgttacc |
| | | gatgtgcgtactgtcccgtggatgccttccgaacgtgagcatctggcgt |
| | | tgagcgcgttactggaggcgttgcctgaacctgtgctctctgtcgatat |
| | | gaaaagcaaagtggatatggcgaacccggcgagctgtcagctttttg |
| | | ggcaaaaattggatcgcctgcgcaaccataccgccgcacaattgatt |
| | | aacggctttaattttttacgttggctggaaagcgaaccgcaagattcgc |
| | | ataacgagcatgtcgttattaatgggcagaatttcctgatggagattac |
| | | gcctgtttatcttcaggatgaaaatgatcaacacgtcctgaccggtgc |
| | | ggtggtgatgttgcgatcaacgattcgtatgggccgccagttgcaaaa |
| | | tgtcgccgcccaggacgtcagcgccttcagtcaaattgtcgccgtca |
| | | gcccgaaaatgaagcatgttgtcgaacaggcgcagaaactggcga |
| | | tgctaagcgcgccgctgctgattacgggtgacacaggtacaggtaa |
| | | agatctctttgcctacgcctgccatcaggcaagccccagagcgggc |
| | | aaaccttacctggcgctgaactgtgcgtctataccggaagatgcggtc |
| | | gagagtcaactgtttggtcatgctccggaagggaagaaaggattcttt |
| | | gagcaggcgaacggtggttcggtgctgttggatgaaataggggaaa |
| | | tgtcaccacggatgcaggcgaaattactgcgtttccttaatgatggca |
| | | ctttccgtcgggttggcgaagaccatgaggtgcatgtcgatgtgcggg |
| | | tgatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| 15 | tyrR_E274Q_T14V | ATGcgtctggaagtcttttgtgaagaccgactcggtctggggcgcga |
| | | attactcgatctactcgtgctaagaggcattgatttacgcggtattgaga |
| | | ttgatcccattgggcgaatctacctcaattttgctgaactggagtttgag |
| | | agtttcagcagtctgatggccgaaatacgccgtattgcgggtgttacc |
| | | gatgtgcgtactgtcccgtggatgccttccgaacgtgagcatctggcgt |
| | | tgagcgcgttactggaggcgttgcctgaacctgtgctctctgtcgatat |
| | | gaaaagcaaagtggatatggcgaacccggcgagctgtcagctttttg |
| | | ggcaaaaattggatcgcctgcgcaaccataccgccgcacaattgatt |
| | | aacggctttaattttttacgttggctggaaagcgaaccgcaagattcgc |
| | | ataacgagcatgtcgttattaatgggcagaatttcctgatggagattac |
| | | gcctgtttatcttcaggatgaaaatgatcaacacgtcctgaccggtgc |
| | | ggtggtgatgttgcgatcaacgattcgtatgggccgccagttgcaaaa |
| | | tgtcgccgcccaggacgtcagcgccttcagtcaaattgtcgccgtca |
| | | gcccgaaaatgaagcatgttgtcgaacaggcgcagaaactggcga |
| | | tgctaagcgcgccgctgctgattacgggtgacacaggtacaggtaa |
| | | agatctctttgcctacgcctgccatcaggcaagccccagagcgggc |
| | | aaaccttacctggcgctgaactgtgcgtctataccggaagatgcggtc |
| | | gagagtcaactgtttggtcatgctccggaagggaagaaaggattcttt |
| | | gagcaggcgaacggtggttcggtgctgttggatgaaataggggaaa |
| | | tgtcaccacggatgcaggcgaaattactgcgtttccttaatgatggca |
| | | ctttccgtcgggttggcgaagaccatgaggtgcatgtcgatgtgcggg |
| | | tgatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| 16 | tyrR_E274Q_D103H | ATGcgtctggaagtcttttgtgaagaccgactcggtctgacccgcga |
| | | attactcgatctactcgtgctaagaggcattgatttacgcggtattgaga |
| | | ttgatcccattgggcgaatctacctcaattttgctgaactggagtttgag |
| | | agtttcagcagtctgatggccgaaatacgccgtattgcgggtgttacc |
| | | gatgtgcgtactgtcccgtggatgccttccgaacgtgagcatctggcgt |
| | | tgagcgcgttactggaggcgttgcctgaacctgtgctctctgtcgatat |
| | | gaaaagcaaagtgcacatggcgaacccggcgagctgtcagcttttt |
| | | gggcaaaaattggatcgcctgcgcaaccataccgccgcacaattga |
| | | ttaacggctttaattttttacgttggctggaaagcgaaccgcaagattcg |
| | | cataacgagcatgtcgttattaatgggcagaatttcctgatggagatta |
| | | cgcctgtttatcttcaggatgaaaatgatcaacacgtcctgaccggtgc |
| | | ggtggtgatgttgcgatcaacgattcgtatgggccgccagttgcaaaa |
| | | tgtcgccgcccaggacgtcagcgccttcagtcaaattgtcgccgtca |
| | | gcccgaaaatgaagcatgttgtcgaacaggcgcagaaactggcga |
| | | tgctaagcgcgccgctgctgattacgggtgacacaggtacaggtaa |
| | | agatctctttgcctacgcctgccatcaggcaagccccagagcgggc |
| | | aaaccttacctggcgctgaactgtgcgtctataccggaagatgcggtc |
| | | gagagtcaactgtttggtcatgctccggaagggaagaaaggattcttt |
| | | gagcaggcgaacggtggttcggtgctgttggatgaaataggggaaa |
| | | tgtcaccacggatgcaggcgaaattactgcgtttccttaatgatggca |
| | | ctttccgtcgggttggcgaagaccatgaggtgcatgtcgatgtgcggg |
| | | tgatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| 17 | tyrR_E274Q_D103S | ATGcgtctggaagtcttttgtgaagaccgactcggtctgacccgcga |
| | | attactcgatctactcgtgctaagaggcattgatttacgcggtattgaga |
| | | ttgatcccattgggcgaatctacctcaattttgctgaactggagtttgag |
| | | agtttcagcagtctgatggccgaaatacgccgtattgcgggtgttacc |
| | | gatgtgcgtactgtcccgtggatgccttccgaacgtgagcatctggcgt |
| | | tgagcgcgttactggaggcgttgcctgaacctgtgctctctgtcgatat |
| | | gaaaagcaaagtgtccatggcgaacccggcgagctgtcagctttttg |
| | | ggcaaaaattggatcgcctgcgcaaccataccgccgcacaattgatt |
| | | aacggctttaattttttacgttggctggaaagcgaaccgcaagattcgc |
| | | ataacgagcatgtcgttattaatgggcagaatttcctgatggagattac |
| | | gcctgtttatcttcaggatgaaaatgatcaacacgtcctgaccggtgc |
| | | ggtggtgatgttgcgatcaacgattcgtatgggccgccagttgcaaaa |
| | | tgtcgccgcccaggacgtcagcgccttcagtcaaattgtcgccgtca |
| | | gcccgaaaatgaagcatgttgtcgaacaggcgcagaaactggcga |
| | | tgctaagcgcgccgctgctgattacgggtgacacaggtacaggtaa |
| | | agatctctttgcctacgcctgccatcaggcaagccccagagcgggc |
| | | aaaccttacctggcgctgaactgtgcgtctataccggaagatgcggtc |
| | | gagagtcaactgtttggtcatgctccggaagggaagaaaggattcttt |
| | | gagcaggcgaacggtggttcggtgctgttggatgaaataggggaaa |
| | | tgtcaccacggatgcaggcgaaattactgcgtttccttaatgatggca |
| | | ctttccgtcgggttggcgaagaccatgaggtgcatgtcgatgtgcggg |
| | | tgatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| 18 | tyrR_E274Q_C7V | ATGcgtctggaagtctttgttgaagaccgactcggtctgacccgcga |
| | | attactcgatctactcgtgctaagaggcattgatttacgcggtattgaga |
| | | ttgatcccattgggcgaatctacctcaattttgctgaactggagtttgag |
| | | agtttcagcagtctgatggccgaaatacgccgtattgcgggtgttacc |
| | | gatgtgcgtactgtcccgtggatgccttccgaacgtgagcatctggcgt |
| | | tgagcgcgttactggaggcgttgcctgaacctgtgctctctgtcgatat |
| | | gaaaagcaaagtggatatggcgaacccggcgagctgtcagctttttg |
| | | ggcaaaaattggatcgcctgcgcaaccataccgccgcacaattgatt |
| | | aacggctttaattttttacgttggctggaaagcgaaccgcaagattcgc |
| | | ataacgagcatgtcgttattaatgggcagaatttcctgatggagattac |
| | | gcctgtttatcttcaggatgaaaatgatcaacacgtcctgaccggtgc |
| | | ggtggtgatgttgcgatcaacgattcgtatgggccgccagttgcaaaa |
| | | tgtcgccgcccaggacgtcagcgccttcagtcaaattgtcgccgtca |
| | | gcccgaaaatgaagcatgttgtcgaacaggcgcagaaactggcga |
| | | tgctaagcgcgccgctgctgattacgggtgacacaggtacaggtaa |
| | | agatctctttgcctacgcctgccatcaggcaagccccagagcgggc |
| | | aaaccttacctggcgctgaactgtgcgtctataccggaagatgcggtc |
| | | gagagtcaactgtttggtcatgctccggaagggaagaaaggattcttt |
| | | gagcaggcgaacggtggttcggtgctgttggatgaaataggggaaa |
| | | tgtcaccacggatgcaggcgaaattactgcgtttccttaatgatggca |
| | | ctttccgtcgggttggcgaagaccatgaggtgcatgtcgatgtgcggg |
| | | tgatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| 19 | tyrR_R10F | ATGcgtctggaagtcttttgtgaagactttctcggtctgacccgcgaat |
| | | tactcgatctactcgtgctaagaggcattgatttacgcggtattgagatt |
| | | gatcccattgggcgaatctacctcaattttgctgaactggagtttgaga |
| | | gtttcagcagtctgatggccgaaatacgccgtattgcgggtgttaccg |
| | | atgtgcgtactgtcccgtggatgccttccgaacgtgagcatctggcgtt |
| | | gagcgcgttactggaggcgttgcctgaacctgtgctctctgtcgatatg |
| | | aaaagcaaagtggatatggcgaacccggcgagctgtcagctttttgg |
| | | gcaaaaattggatcgcctgcgcaaccataccgccgcacaattgatta |
| | | acggctttaattttttacgttggctggaaagcgaaccgcaagattcgca |
| | | taacgagcatgtcgttattaatgggcagaatttcctgatggagattacg |
| | | cctgtttatcttcaggatgaaaatgatcaacacgtcctgaccggtgcg |
| | | gtggtgatgttgcgatcaacgattcgtatgggccgccagttgcaaaat |
| | | gtcgccgcccaggacgtcagcgccttcagtcaaattgtcgccgtcag |
| | | cccgaaaatgaagcatgttgtcgaacaggcgcagaaactggcgat |
| | | gctaagcgcgccgctgctgattacgggtgacacaggtacaggtaaa |
| | | gatctctttgcctacgcctgccatcaggcaagccccagagcgggca |
| | | aaccttacctggcgctgaactgtgcgtctataccggaagatgcggtcg |
| | | agagtgaactgtttggtcatgctccggaagggaagaaaggattctttg |
| | | agcaggcgaacggtggttcggtgctgttggatgaaataggggaaat |
| | | gtcaccacggatgcaggcgaaattactgcgtttccttaatgatggcac |
| | | tttccgtcgggttggcgaagaccatgaggtgcatgtcgatgtgcgggt |
| | | gatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| 20 | tyrR_L11G | ATGcgtctggaagtcttttgtgaagaccgagggggtctgacccgcg |
| | | aattactcgatctactcgtgctaagaggcattgatttacgcggtattgag |
| | | attgatcccattgggcgaatctacctcaattttgctgaactggagtttga |
| | | gagtttcagcagtctgatggccgaaatacgccgtattgcgggtgttac |
| | | cgatgtgcgtactgtcccgtggatgccttccgaacgtgagcatctggc |
| | | gttgagcgcgttactggaggcgttgcctgaacctgtgctctctgtcgat |
| | | atgaaaagcaaagtggatatggcgaacccggcgagctgtcagctttt |
| | | tgggcaaaaattggatcgcctgcgcaaccataccgccgcacaattg |
| | | attaacggctttaattttttacgttggctggaaagcgaaccgcaagattc |
| | | gcataacgagcatgtcgttattaatgggcagaatttcctgatggagatt |
| | | acgcctgtttatcttcaggatgaaaatgatcaacacgtcctgaccggt |
| | | gcggtggtgatgttgcgatcaacgattcgtatgggccgccagttgcaa |
| | | aatgtcgccgcccaggacgtcagcgccttcagtcaaattgtcgccgt |
| | | cagcccgaaaatgaagcatgttgtcgaacaggcgcagaaactggc |
| | | gatgctaagcgcgccgctgctgattacgggtgacacaggtacaggt |
| | | aaagatctctttgcctacgcctgccatcaggcaagccccagagcgg |
| | | gcaaaccttacctggcgctgaactgtgcgtctataccggaagatgcg |
| | | gtcgagagtgaactgtttggtcatgctccggaagggaagaaaggatt |
| | | ctttgagcaggcgaacggtggttcggtgctgttggatgaaatagggg |
| | | aaatgtcaccacggatgcaggcgaaattactgcgtttccttaatgatg |
| | | gcactttccgtcgggttggcgaagaccatgaggtgcatgtcgatgtgc |
| | | gggtgatttgcgctacgcagaagaatctggtcgaactggtgcaaaaa |
| | | ggcatgttccgtgaagatctctattatcgtctgaacgtgttgacgctcaa |
| | | tctgccgccgctacgtgactgtccgcaggacatcatgccgttaactga |
| | | gctgttcgtcgcccgctttgccgacgagcagggcgtgccgcgtccga |
| | | aactggccgctgacctgaatactgtacttacgcgttatgcgtggccgg |
| | | gaaatgtgcggcagttaaagaacgctatctatcgcgcactgacaca |
| | | actggacggttatgagctgcgtccacaggatattttgttgccggattatg |
| | | acgccgcaacggtagccgtgggcgaagatgcgatggaaggttcgc |
| | | tggacgaaatcaccagccgttttgaacgctcggtattaacccagcttta |
| | | tcgcaattatcccagcacgcgcaaactggcaaaacgtctcggcgttt |
| | | cacataccgcgattgccaataagttgcgggaatatggtctgagtcag |
| | | aagaagaacgaagagtaa |
| 21 | tyrR_G12T | ATGcgtctggaagtcttttgtgaagaccgactcacgctgacccgcg |
| | | aattactcgatctactcgtgctaagaggcattgatttacgcggtattgag |
| | | attgatcccattgggcgaatctacctcaattttgctgaactggagtttga |
| | | gagtttcagcagtctgatggccgaaatacgccgtattgcgggtgttac |
| | | cgatgtgcgtactgtcccgtggatgccttccgaacgtgagcatctggc |
| | | gttgagcgcgttactggaggcgttgcctgaacctgtgctctctgtcgat |
| | | atgaaaagcaaagtggatatggcgaacccggcgagctgtcagctttt |
| | | tgggcaaaaattggatcgcctgcgcaaccataccgccgcacaattg |
| | | attaacggctttaattttttacgttggctggaaagcgaaccgcaagattc |
| | | gcataacgagcatgtcgttattaatgggcagaatttcctgatggagatt |
| | | acgcctgtttatcttcaggatgaaaatgatcaacacgtcctgaccggt |
| | | gcggtggtgatgttgcgatcaacgattcgtatgggccgccagttgcaa |
| | | aatgtcgccgcccaggacgtcagcgccttcagtcaaattgtcgccgt |
| | | cagcccgaaaatgaagcatgttgtcgaacaggcgcagaaactggc |
| | | gatgctaagcgcgccgctgctgattacgggtgacacaggtacaggt |
| | | aaagatctctttgcctacgcctgccatcaggcaagccccagagcgg |
| | | gcaaaccttacctggcgctgaactgtgcgtctataccggaagatgcg |
| | | gtcgagagtgaactgtttggtcatgctccggaagggaagaaaggatt |
| | | ctttgagcaggcgaacggtggttcggtgctgttggatgaaatagggg |
| | | aaatgtcaccacggatgcaggcgaaattactgcgtttccttaatgatg |
| | | gcactttccgtcgggttggcgaagaccatgaggtgcatgtcgatgtgc |
| | | gggtgatttgcgctacgcagaagaatctggtcgaactggtgcaaaaa |
| | | ggcatgttccgtgaagatctctattatcgtctgaacgtgttgacgctcaa |
| | | tctgccgccgctacgtgactgtccgcaggacatcatgccgttaactga |
| | | gctgttcgtcgcccgctttgccgacgagcagggcgtgccgcgtccga |
| | | aactggccgctgacctgaatactgtacttacgcgttatgcgtggccgg |
| | | gaaatgtgcggcagttaaagaacgctatctatcgcgcactgacaca |
| | | actggacggttatgagctgcgtccacaggatattttgttgccggattatg |
| | | acgccgcaacggtagccgtgggcgaagatgcgatggaaggttcgc |
| | | tggacgaaatcaccagccgttttgaacgctcggtattaacccagcttta |
| | | tcgcaattatcccagcacgcgcaaactggcaaaacgtctcggcgttt |
| | | cacataccgcgattgccaataagttgcgggaatatggtctgagtcag |
| | | aagaagaacgaagagtaa |
| 22 | tyrR_L13G | ATGcgtctggaagtcttttgtgaagaccgactcggtggaacccgcg |
| | | aattactcgatctactcgtgctaagaggcattgatttacgcggtattgag |
| | | attgatcccattgggcgaatctacctcaattttgctgaactggagtttga |
| | | gagtttcagcagtctgatggccgaaatacgccgtattgcgggtgttac |
| | | cgatgtgcgtactgtcccgtggatgccttccgaacgtgagcatctggc |
| | | gttgagcgcgttactggaggcgttgcctgaacctgtgctctctgtcgat |
| | | atgaaaagcaaagtggatatggcgaacccggcgagctgtcagctttt |
| | | tgggcaaaaattggatcgcctgcgcaaccataccgccgcacaattg |
| | | attaacggctttaattttttacgttggctggaaagcgaaccgcaagattc |
| | | gcataacgagcatgtcgttattaatgggcagaatttcctgatggagatt |
| | | acgcctgtttatcttcaggatgaaaatgatcaacacgtcctgaccggt |
| | | gcggtggtgatgttgcgatcaacgattcgtatgggccgccagttgcaa |
| | | aatgtcgccgcccaggacgtcagcgccttcagtcaaattgtcgccgt |
| | | cagcccgaaaatgaagcatgttgtcgaacaggcgcagaaactggc |
| | | gatgctaagcgcgccgctgctgattacgggtgacacaggtacaggt |
| | | aaagatctctttgcctacgcctgccatcaggcaagccccagagcgg |
| | | gcaaaccttacctggcgctgaactgtgcgtctataccggaagatgcg |
| | | gtcgagagtgaactgtttggtcatgctccggaagggaagaaaggatt |
| | | ctttgagcaggcgaacggtggttcggtgctgttggatgaaatagggg |
| | | aaatgtcaccacggatgcaggcgaaattactgcgtttccttaatgatg |
| | | gcactttccgtcgggttggcgaagaccatgaggtgcatgtcgatgtgc |
| | | gggtgatttgcgctacgcagaagaatctggtcgaactggtgcaaaaa |
| | | ggcatgttccgtgaagatctctattatcgtctgaacgtgttgacgctcaa |
| | | tctgccgccgctacgtgactgtccgcaggacatcatgccgttaactga |
| | | gctgttcgtcgcccgctttgccgacgagcagggcgtgccgcgtccga |
| | | aactggccgctgacctgaatactgtacttacgcgttatgcgtggccgg |
| | | gaaatgtgcggcagttaaagaacgctatctatcgcgcactgacaca |
| | | actggacggttatgagctgcgtccacaggatattttgttgccggattatg |
| | | acgccgcaacggtagccgtgggcgaagatgcgatggaaggttcgc |
| | | tggacgaaatcaccagccgttttgaacgctcggtattaacccagcttta |
| | | tcgcaattatcccagcacgcgcaaactggcaaaacgtctcggcgttt |
| | | cacataccgcgattgccaataagttgcgggaatatggtctgagtcag |
| | | aagaagaacgaagagtaa |
| 23 | tyrR_T14K | ATGcgtctggaagtcttttgtgaagaccgactcggtctgaagcgcga |
| | | attactcgatctactcgtgctaagaggcattgatttacgcggtattgaga |
| | | ttgatcccattgggcgaatctacctcaattttgctgaactggagtttgag |
| | | agtttcagcagtctgatggccgaaatacgccgtattgcgggtgttacc |
| | | gatgtgcgtactgtcccgtggatgccttccgaacgtgagcatctggcgt |
| | | tgagcgcgttactggaggcgttgcctgaacctgtgctctctgtcgatat |
| | | gaaaagcaaagtggatatggcgaacccggcgagctgtcagctttttg |
| | | ggcaaaaattggatcgcctgcgcaaccataccgccgcacaattgatt |
| | | aacggctttaattttttacgttggctggaaagcgaaccgcaagattcgc |
| | | ataacgagcatgtcgttattaatgggcagaatttcctgatggagattac |
| | | gcctgtttatcttcaggatgaaaatgatcaacacgtcctgaccggtgc |
| | | ggtggtgatgttgcgatcaacgattcgtatgggccgccagttgcaaaa |
| | | tgtcgccgcccaggacgtcagcgccttcagtcaaattgtcgccgtca |
| | | gcccgaaaatgaagcatgttgtcgaacaggcgcagaaactggcga |
| | | tgctaagcgcgccgctgctgattacgggtgacacaggtacaggtaa |
| | | agatctctttgcctacgcctgccatcaggcaagccccagagcgggc |
| | | aaaccttacctggcgctgaactgtgcgtctataccggaagatgcggtc |
| | | gagagtgaactgtttggtcatgctccggaagggaagaaaggattcttt |
| | | gagcaggcgaacggtggttcggtgctgttggatgaaataggggaaa |
| | | tgtcaccacggatgcaggcgaaattactgcgtttccttaatgatggca |
| | | ctttccgtcgggttggcgaagaccatgaggtgcatgtcgatgtgcggg |
| | | tgatttgcgctacgcagaagaatctggtcgaactggtgcaaaaaggc |
| | | atgttccgtgaagatctctattatcgtctgaacgtgttgacgctcaatctg |
| | | ccgccgctacgtgactgtccgcaggacatcatgccgttaactgagct |
| | | gttcgtcgcccgctttgccgacgagcagggcgtgccgcgtccgaaa |
| | | ctggccgctgacctgaatactgtacttacgcgttatgcgtggccggga |
| | | aatgtgcggcagttaaagaacgctatctatcgcgcactgacacaact |
| | | ggacggttatgagctgcgtccacaggatattttgttgccggattatgac |
| | | gccgcaacggtagccgtgggcgaagatgcgatggaaggttcgctg |
| | | gacgaaatcaccagccgttttgaacgctcggtattaacccagctttatc |
| | | gcaattatcccagcacgcgcaaactggcaaaacgtctcggcgtttca |
| | | cataccgcgattgccaataagttgcgggaatatggtctgagtcagaa |
| | | gaagaacgaagagtaa |
| | TynA-FeaR | |
| | Sensor | |
| 24 | PtynA (used in | tcggtgaatgaagggcaacggcgaatagttgcccttttatttcactaag |
| | pHJY028) | ttttgtgacgttgtcacattatgcatgatgtgtacatctattttcagggcatc |
| | | cactgtatgaaaagctggcacacctgccaaacaacctggcaggtgc |
| | | aggcaatcccctttgcatcagtactgataatgtgaacctgactaaacc |
| | | gcccacagagcgcggttgctaacaagaacacaacagaaagagg |
| | | ggttaata |
| 25 | PtynA (used in | tatgaaaagctggcacacctgccaaacaacctggcaggtgcaggc |
| | pCX008) | aatcccctttgcatcagtactgataatgtgaacctgactaaaccgccc |
| | | acagagcgcggttgctaacaagaacacaacagaaagaggggtta |
| | | ata |
| 26 | gfp RBS library for | AgaacacaacaRRRRRRRRRgttaataATG |
| | PtynA-gfp reporter | |
| 27 | Optimal RBS for | agaacacaacagaaagagggttaataATG |
| | PtynA-gfp reporter | |
| | (pHJY028) | |
| 28 | bla RBS library | aactaagacgWtctRggBgtcaWHaaATG |
| 29 | RBS-bla | aactaagacgttctaggcgtcatcaaATGagtattcaacatttccgtg |
| | | tcgcccttattcccttttttgcggcattttgccttcctgtttttgctcacccaga |
| | | aacgctggtgaaagtaaaagatgctgaagatcagttgggtgcacga |
| | | gtgggttacatcgaactggatctcaacagcggtaagatccttgagagt |
| | | tttcgccccgaagaacgttttccaatgatgagcacttttaaagttctgct |
| | | atgtggcgcggtattatcccgtattgacgccgggcaagagcaactcg |
| | | gtcgccgcatacactattctcagaatgacttggttgagtactcaccagt |
| | | cacagaaaagcatcttacggatggcatgacagtaagagaattatgc |
| | | agtgctgccataaccatgagtgataacactgcggccaacttacttctg |
| | | acaacgatcggaggaccgaaggagctaaccgcttttttgcacaaca |
| | | tgggggatcatgtaactcgccttgatcgttgggaaccggagctgaatg |
| | | aagccataccaaacgacgagcgtgacaccacgatgcctgtagcaa |
| | | tggcaacaacgttgcgcaaactattaactggcgaactacttactctag |
| | | cttcccggcaacaattaatagactggatggaggcggataaagttgca |
| | | ggaccacttctgcgctcggcccttccggctggctggtttattgctgataa |
| | | atctggagccggtgagcgtgggtctcgcggtatcattgcagcactgg |
| | | ggccagatggtaagccctcccgtatcgtagttatctacacgacgggg |
| | | agtcaggcaactatggatgaacgaaatagacagatcgctgagata |
| | | ggtgcctcactgattaagcattggtaa |
| 30 | sacB RBS library | caaaatccWcaaDggtRgttgBBgATG |
| 31 | RBS-sacB | caaaatcctcaaaggtggttgcggATGaacatcaaaaagtttgca |
| | | aaacaagcaacagtattaacctttactaccgcactgctggcaggag |
| | | gcgcaactcaagcgtttgcgaaagaaacgaaccaaaagccatata |
| | | aggaaacatacggcatttcccatattacacgccatgatatgctgcaa |
| | | atccctgaacagcaaaaaaatgaaaaatatcaagttcctgaattcga |
| | | ttcgtccacaattaaaaatatctcttctgcaaaaggcctggacgtttgg |
| | | gacagctggccattacaaaacgctgacggcactgtcgcaaactatc |
| | | acggctaccacatcgtctttgcattagccggagatcctaaaaatgcgg |
| | | atgacacatcgatttacatgttctatcaaaaagtcggcgaaacttctatt |
| | | gacagctggaaaaacgctggccgcgtctttaaagacagcgacaaa |
| | | ttcgatgcaaatgattctatcctaaaagaccaaacacaagaatggtc |
| | | aggttcagccacatttacatctgacggaaaaatccgtttattctacact |
| | | gatttctccggtaaacattacggcaaacaaacactgacaactgcaca |
| | | agttaacgtatcagcatcagacagctctttgaacatcaacggtgtaga |
| | | ggattataaatcaatctttgacggtgacggaaaaacgtatcaaaatgt |
| | | acagcagttcatcgatgaaggcaactacagctcaggcgacaaccat |
| | | acgctgagagatcctcactacgtagaagataaaggccacaaatact |
| | | tagtatttgaagcaaacactggaactgaagatggctaccaaggcga |
| | | agaatctttatttaacaaagcatactatggcaaaagcacatcattcttc |
| | | cgtcaagaaagtcaaaaacttctgcaaagcgataaaaaacgcacg |
| | | gctgagttagcaaacggcgctctcggtatgattgagctaaacgatgat |
| | | tacacactgaaaaaagtgatgaaaccgctgattgcatctaacacagt |
| | | aacagatgaaattgaacgcgcgaacgtctttaaaatgaacggcaaa |
| | | tggtatctgttcactgactcccgcggatcaaaaatgacgattgacggc |
| | | attacgtctaacgatatttacatgcttggttatgtttctaattctttaactggc |
| | | ccatacaagccgctgaacaaaactggccttgtgttaaaaatggatctt |
| | | gatcctaacgatgtaacctttacttactcacacttcgctgtacctcaagc |
| | | gaaaggaaacaatgtcgtgattacaagctatatgacaaacagagg |
| | | attctacgcagacaaacaatcaacgtttgcgccaagcttcctgctgaa |
| | | catcaaaggcaagaaaacatctgttgtcaaagacagcatccttgaa |
| | | caaggacaattaacagttaacaaataa |
| 32 | [Pcon-RBS-]tynA | [Ttgacagctagctcagtcctaggagttgagctagccttgcccacag |
| | | agcgcggttgctaacaagaacacaacatctgacgaggttaata]AT |
| | | Gggaagcccctctctgtattctgcccgtaaaacaaccctggcgttgg |
| | | cagtcgccttaagtttcgcctggcaagcgccggtatttgcccacggtg |
| | | gtgaagcgcatatggtgccaatggataaaacgcttaaagaatttggt |
| | | gccgatgtgcagtgggacgactacgcccagctctttaccctgattaaa |
| | | gatggcgcgtacgtgaaagtgaagcctggtgcgcaaacagcaattg |
| | | ttaatggtcagcctctggcactgcaagtaccggtagtgatgaaagac |
| | | aataaagcctgggtttctgacacctttattaacgatgttttccagtccgg |
| | | gctggatcaaacctttcaggtagaaaagcgccctcacccacttaatg |
| | | cgctaactgcggacgaaattaaacaggccgttgaaattgttaaagctt |
| | | ccgcggacttcaaacccaatacccgttttactgagatctccctgctacc |
| | | gccagataaagaagctgtctgggcgtttgcgctggaaaacaaaccg |
| | | gttgaccagccgcgcaaagccgacgtcattatgctcgacggcaaac |
| | | atatcatcgaagcggtggtggatctgcaaaacaacaaactgctctcc |
| | | tggcaacccattaaagacgcccacggtatggtgttgctggatgatttc |
| | | gccagtgtgcagaacattattaacaacagtgaagaatttgccgctgc |
| | | cgtgaagaaacgcggtattactgatgcgaaaaaagtgattaccacg |
| | | ccgctgaccgtaggttatttcgatggtaaagatggcctgaaacaagat |
| | | gcccggttgctcaaagtcatcagctatcttgatgtcggtgatggcaact |
| | | actgggcacatcccatcgaaaacctggtggcggtcgttgatttagaa |
| | | cagaaaaaaatcgttaagattgaagaaggtccggtagttccggtgcc |
| | | aatgaccgcacgcccatttgatggccgtgaccgcgttgctccggcag |
| | | ttaagcctatgcaaatcattgagcctgaaggtaaaaattacaccatta |
| | | ctggcgatatgattcactggcggaactgggattttcacctcagcatga |
| | | actctcgcgtcgggccgatgatctccaccgtgacttataacgacaatg |
| | | gcaccaaacgcaaagtcatgtacgaaggttctctcggcggcatgatt |
| | | gtgccttacggtgatcctgatattggctggtactttaaagcgtatctgga |
| | | ctctggtgactacggtatgggcacgctaacctcaccaattgctcgtggt |
| | | aaagatgccccgtctaacgcagtgctccttaatgaaaccatcgccga |
| | | ctacactggcgtgccgatggagatccctcgcgctatcgcggtatttga |
| | | acgttatgccgggccggagtataagcatcaggaaatgggccagcc |
| | | caacgtcagtaccgaacgccgggagttagtggtgcgctggatcagt |
| | | acagtgggtaactatgactacatttttgactggatcttccatgaaaacg |
| | | gcactattggcatcgatgccggtgctacgggcatcgaagcggtgaa |
| | | aggtgttaaagcgaaaaccatgcacgatgagacggcgaaagatg |
| | | acacgcgctacggcacgcttatcgatcacaatatcgtgggtactaca |
| | | caccaacatatttataatttccgcctcgatctggatgtagatggcgaga |
| | | ataacagcctggtggcgatggacccagtggtaaaaccgaatactgc |
| | | cggtggcccacgcaccagtaccatgcaagttaatcagtacaacatc |
| | | ggcaatgaacaggatgccgcacagaaatttgatccgggcacgattc |
| | | gtctgttgagtaacccgaacaaagagaaccgcatgggcaatccggt |
| | | ttcctatcaaattattccttatgcaggtggtactcacccggtagcaaaa |
| | | ggtgcccagttcgcgccggacgagtggatctatcatcgtttaagcttta |
| | | tggacaagcagctctgggtaacgcgttatcatcctggcgagcgtttcc |
| | | cggaaggcaaatatccgaaccgttctactcatgacaccggtcttgga |
| | | caatacagtaaggataacgagtcgctggacaacaccgacgccgtt |
| | | gtctggatgaccaccggcaccacacatgtggcccgcgccgaagag |
| | | tggccgattatgccgaccgaatgggtacatactctgctgaaaccatg |
| | | gaacttctttgacgaaacgccaacgctaggggcgctgaagaaagat |
| | | aagtga |
| 33 | tynA_G494S | ATGggaagcccctctctgtattctgcccgtaaaacaaccctggcgtt |
| | | ggcagtcgccttaagtttcgcctggcaagcgccggtatttgcccacgg |
| | | tggtgaagcgcatatggtgccaatggataaaacgcttaaagaatttg |
| | | gtgccgatgtgcagtgggacgactacgcccagctctttaccctgatta |
| | | aagatggcgcgtacgtgaaagtgaagcctggtgcgcaaacagcaa |
| | | ttgttaatggtcagcctctggcactgcaagtaccggtagtgatgaaag |
| | | acaataaagcctgggtttctgacacctttattaacgatgttttccagtcc |
| | | gggctggatcaaacctttcaggtagaaaagcgccctcacccacttaa |
| | | tgcgctaactgcggacgaaattaaacaggccgttgaaattgttaaag |
| | | cttccgcggacttcaaacccaatacccgttttactgagatctccctgcta |
| | | ccgccagataaagaagctgtctgggcgtttgcgctggaaaacaaac |
| | | cggttgaccagccgcgcaaagccgacgtcattatgctcgacggcaa |
| | | acatatcatcgaagcggtggtggatctgcaaaacaacaaactgctct |
| | | cctggcaacccattaaagacgcccacggtatggtgttgctggatgatt |
| | | tcgccagtgtgcagaacattattaacaacagtgaagaatttgccgctg |
| | | ccgtgaagaaacgcggtattactgatgcgaaaaaagtgattaccac |
| | | gccgctgaccgtaggttatttcgatggtaaagatggcctgaaacaag |
| | | atgcccggttgctcaaagtcatcagctatcttgatgtcggtgatggcaa |
| | | ctactgggcacatcccatcgaaaacctggtggcggtcgttgatttaga |
| | | acagaaaaaaatcgttaagattgaagaaggtccggtagttccggtg |
| | | ccaatgaccgcacgcccatttgatggccgtgaccgcgttgctccggc |
| | | agttaagcctatgcaaatcattgagcctgaaggtaaaaattacaccat |
| | | tactggcgatatgattcactggcggaactgggattttcacctcagcatg |
| | | aactctcgcgtcgggccgatgatctccaccgtgacttataacgacaat |
| | | ggcaccaaacgcaaagtcatgtacgaaggttctctcggcggcatga |
| | | ttgtgccttacggtgatcctgatattggctggtactttaaagcgtatctgg |
| | | actctggtgactacggtatgggcacgctaacctcaccaattgctcgtg |
| | | gtaaagatgccccgtctaacgcagtgctccttaatgaaaccatcgcc |
| | | gactacactggcgtgccgatggagatccctcgcgctatcgcggtattt |
| | | gaacgttatgccgggccggagtataagcatcaggaaatgggccag |
| | | cccaacgtcagtaccgaacgccgggagttagtggtgcgctggatca |
| | | gtacagtgtccaactatgactacatttttgactggatcttccatgaaaac |
| | | ggcactattggcatcgatgccggtgctacgggcatcgaagcggtga |
| | | aaggtgttaaagcgaaaaccatgcacgatgagacggcgaaagat |
| | | gacacgcgctacggcacgcttatcgatcacaatatcgtgggtactac |
| | | acaccaacatatttataatttccgcctcgatctggatgtagatggcgag |
| | | aataacagcctggtggcgatggacccagtggtaaaaccgaatactg |
| | | ccggtggcccacgcaccagtaccatgcaagttaatcagtacaacat |
| | | cggcaatgaacaggatgccgcacagaaatttgatccgggcacgatt |
| | | cgtctgttgagtaacccgaacaaagagaaccgcatgggcaatccg |
| | | gtttcctatcaaattattccttatgcaggtggtactcacccggtagcaaa |
| | | aggtgcccagttcgcgccggacgagtggatctatcatcgtttaagcttt |
| | | atggacaagcagctctgggtaacgcgttatcatcctggcgagcgtttc |
| | | ccggaaggcaaatatccgaaccgttctactcatgacaccggtcttgg |
| | | acaatacagtaaggataacgagtcgctggacaacaccgacgccgt |
| | | tgtctggatgaccaccggcaccacacatgtggcccgcgccgaaga |
| | | gtggccgattatgccgaccgaatgggtacatactctgctgaaaccat |
| | | ggaacttctttgacgaaacgccaacgctaggggcgctgaagaaag |
| | | ataagtga |
| 34 | tynA_G415H | ATGggaagcccctctctgtattctgcccgtaaaacaaccctggcgtt |
| | | ggcagtcgccttaagtttcgcctggcaagcgccggtatttgcccacgg |
| | | tggtgaagcgcatatggtgccaatggataaaacgcttaaagaatttg |
| | | gtgccgatgtgcagtgggacgactacgcccagctctttaccctgatta |
| | | aagatggcgcgtacgtgaaagtgaagcctggtgcgcaaacagcaa |
| | | ttgttaatggtcagcctctggcactgcaagtaccggtagtgatgaaag |
| | | acaataaagcctgggtttctgacacctttattaacgatgttttccagtcc |
| | | gggctggatcaaacctttcaggtagaaaagcgccctcacccacttaa |
| | | tgcgctaactgcggacgaaattaaacaggccgttgaaattgttaaag |
| | | cttccgcggacttcaaacccaatacccgttttactgagatctccctgcta |
| | | ccgccagataaagaagctgtctgggcgtttgcgctggaaaacaaac |
| | | cggttgaccagccgcgcaaagccgacgtcattatgctcgacggcaa |
| | | acatatcatcgaagcggtggtggatctgcaaaacaacaaactgctct |
| | | cctggcaacccattaaagacgcccacggtatggtgttgctggatgatt |
| | | tcgccagtgtgcagaacattattaacaacagtgaagaatttgccgctg |
| | | ccgtgaagaaacgcggtattactgatgcgaaaaaagtgattaccac |
| | | gccgctgaccgtaggttatttcgatggtaaagatggcctgaaacaag |
| | | atgcccggttgctcaaagtcatcagctatcttgatgtcggtgatggcaa |
| | | ctactgggcacatcccatcgaaaacctggtggcggtcgttgatttaga |
| | | acagaaaaaaatcgttaagattgaagaaggtccggtagttccggtg |
| | | ccaatgaccgcacgcccatttgatggccgtgaccgcgttgctccggc |
| | | agttaagcctatgcaaatcattgagcctgaaggtaaaaattacaccat |
| | | tactggcgatatgattcactggcggaactgggattttcacctcagcatg |
| | | aactctcgcgtcgggccgatgatctccaccgtgacttataacgacaat |
| | | ggcaccaaacgcaaagtcatgtacgaaggttctctcggcggcatga |
| | | ttgtgccttacggtgatcctgatattggctggtactttaaagcgtatctgg |
| | | actctcacgactacggtatgggcacgctaacctcaccaattgctcgtg |
| | | gtaaagatgccccgtctaacgcagtgctccttaatgaaaccatcgcc |
| | | gactacactggcgtgccgatggagatccctcgcgctatcgcggtattt |
| | | gaacgttatgccgggccggagtataagcatcaggaaatgggccag |
| | | cccaacgtcagtaccgaacgccgggagttagtggtgcgctggatca |
| | | gtacagtgggtaactatgactacatttttgactggatcttccatgaaaac |
| | | ggcactattggcatcgatgccggtgctacgggcatcgaagcggtga |
| | | aaggtgttaaagcgaaaaccatgcacgatgagacggcgaaagat |
| | | gacacgcgctacggcacgcttatcgatcacaatatcgtgggtactac |
| | | acaccaacatatttataatttccgcctcgatctggatgtagatggcgag |
| | | aataacagcctggtggcgatggacccagtggtaaaaccgaatactg |
| | | ccggtggcccacgcaccagtaccatgcaagttaatcagtacaacat |
| | | cggcaatgaacaggatgccgcacagaaatttgatccgggcacgatt |
| | | cgtctgttgagtaacccgaacaaagagaaccgcatgggcaatccg |
| | | gtttcctatcaaattattccttatgcaggtggtactcacccggtagcaaa |
| | | aggtgcccagttcgcgccggacgagtggatctatcatcgtttaagcttt |
| | | atggacaagcagctctgggtaacgcgttatcatcctggcgagcgtttc |
| | | ccggaaggcaaatatccgaaccgttctactcatgacaccggtcttgg |
| | | acaatacagtaaggataacgagtcgctggacaacaccgacgccgt |
| | | tgtctggatgaccaccggcaccacacatgtggcccgcgccgaaga |
| | | gtggccgattatgccgaccgaatgggtacatactctgctgaaaccat |
| | | ggaacttctttgacgaaacgccaacgctaggggcgctgaagaaag |
| | | ataagtga |
| 35 | tynA_S414M | ATGggaagcccctctctgtattctgcccgtaaaacaaccctggcgtt |
| | | ggcagtcgccttaagtttcgcctggcaagcgccggtatttgcccacgg |
| | | tggtgaagcgcatatggtgccaatggataaaacgcttaaagaatttg |
| | | gtgccgatgtgcagtgggacgactacgcccagctctttaccctgatta |
| | | aagatggcgcgtacgtgaaagtgaagcctggtgcgcaaacagcaa |
| | | ttgttaatggtcagcctctggcactgcaagtaccggtagtgatgaaag |
| | | acaataaagcctgggtttctgacacctttattaacgatgttttccagtcc |
| | | gggctggatcaaacctttcaggtagaaaagcgccctcacccacttaa |
| | | tgcgctaactgcggacgaaattaaacaggccgttgaaattgttaaag |
| | | cttccgcggacttcaaacccaatacccgttttactgagatctccctgcta |
| | | ccgccagataaagaagctgtctgggcgtttgcgctggaaaacaaac |
| | | cggttgaccagccgcgcaaagccgacgtcattatgctcgacggcaa |
| | | acatatcatcgaagcggtggtggatctgcaaaacaacaaactgctct |
| | | cctggcaacccattaaagacgcccacggtatggtgttgctggatgatt |
| | | tcgccagtgtgcagaacattattaacaacagtgaagaatttgccgctg |
| | | ccgtgaagaaacgcggtattactgatgcgaaaaaagtgattaccac |
| | | gccgctgaccgtaggttatttcgatggtaaagatggcctgaaacaag |
| | | atgcccggttgctcaaagtcatcagctatcttgatgtcggtgatggcaa |
| | | ctactgggcacatcccatcgaaaacctggtggcggtcgttgatttaga |
| | | acagaaaaaaatcgttaagattgaagaaggtccggtagttccggtg |
| | | ccaatgaccgcacgcccatttgatggccgtgaccgcgttgctccggc |
| | | agttaagcctatgcaaatcattgagcctgaaggtaaaaattacaccat |
| | | tactggcgatatgattcactggcggaactgggattttcacctcagcatg |
| | | aactctcgcgtcgggccgatgatctccaccgtgacttataacgacaat |
| | | ggcaccaaacgcaaagtcatgtacgaaggttctctcggcggcatga |
| | | ttgtgccttacggtgatcctgatattggctggtactttaaagcgtatctgg |
| | | acatgggtgactacggtatgggcacgctaacctcaccaattgctcgtg |
| | | gtaaagatgccccgtctaacgcagtgctccttaatgaaaccatcgcc |
| | | gactacactggcgtgccgatggagatccctcgcgctatcgcggtattt |
| | | gaacgttatgccgggccggagtataagcatcaggaaatgggccag |
| | | cccaacgtcagtaccgaacgccgggagttagtggtgcgctggatca |
| | | gtacagtgggtaactatgactacatttttgactggatcttccatgaaaac |
| | | ggcactattggcatcgatgccggtgctacgggcatcgaagcggtga |
| | | aaggtgttaaagcgaaaaccatgcacgatgagacggcgaaagat |
| | | gacacgcgctacggcacgcttatcgatcacaatatcgtgggtactac |
| | | acaccaacatatttataatttccgcctcgatctggatgtagatggcgag |
| | | aataacagcctggtggcgatggacccagtggtaaaaccgaatactg |
| | | ccggtggcccacgcaccagtaccatgcaagttaatcagtacaacat |
| | | cggcaatgaacaggatgccgcacagaaatttgatccgggcacgatt |
| | | cgtctgttgagtaacccgaacaaagagaaccgcatgggcaatccg |
| | | gtttcctatcaaattattccttatgcaggtggtactcacccggtagcaaa |
| | | aggtgcccagttcgcgccggacgagtggatctatcatcgtttaagcttt |
| | | atggacaagcagctctgggtaacgcgttatcatcctggcgagcgtttc |
| | | ccggaaggcaaatatccgaaccgttctactcatgacaccggtcttgg |
| | | acaatacagtaaggataacgagtcgctggacaacaccgacgccgt |
| | | tgtctggatgaccaccggcaccacacatgtggcccgcgccgaaga |
| | | gtggccgattatgccgaccgaatgggtacatactctgctgaaaccat |
| | | ggaacttctttgacgaaacgccaacgctaggggcgctgaagaaag |
| | | ataagtga |
| 36 | [Pcon-RBS-]feaR | [ttgacagctagctcagtcctaggtcttgcgctagcgcaacacaaatg |
| | | caacaataaaaatacatttcacagagcgaaaacgtgcc]ATGaac |
| | | cccgcagtggataatgagtttcagcaatggctttcccaaatcaatcag |
| | | gtatgcggaaattttaccggacgcctgctgactgagcgttacacgggt |
| | | gtactggacacgcattttgccaaaggactaaagctgagtaccgtgac |
| | | aaccagcggggtgaatttatcccgcacctggcaggaagtaaaagg |
| | | cagcgacgatgcctggttttacaccgtttttcagcttagtggtcaggca |
| | | ataatggagcaggatgagcgtcaggtgcagattggcgctggcgata |
| | | ttacgttactcgatgcctcacgcccctgttcgctttactggcaggagtctt |
| | | ctaaacagatttcattacttttgccacgcactctgctggaacaatattttc |
| | | cccatcaaaaacctatctgcgcagaaagactggacgctgacttacc |
| | | catggtgcaactcagtcatcgcctgttacaggagagcatgaataatc |
| | | cggcactttctgaaacagaaagtgaagctgcgctacaggcgatggt |
| | | gtgtctgctgcgcccggtacttcatcagcgggaatctgttcaacctcgt |
| | | cgtgaacgtcagtttcaaaaagtggttacgttgatagacgataatattc |
| | | gcgaagagatattacgcccggagtggatagccggagagacaggta |
| | | tgtcagtacgtagtttgtaccgaatgtttgccgataaaggtttggtagtc |
| | | gcgcaatatattcgtaaccgtcgtctcgatttttgtgcagatgcgattcg |
| | | ccatgccgcagatgatgaaaaactggcaggcatcggctttcattggg |
| | | gattttctgaccagagtcatttttcaacggtatttaagcaacgctttggga |
| | | tgacgccaggcgagtatcgacgtaaattccgctaa |
| 37 | feaR_A81T | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagacaataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 38 | feaR_A81L | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagttgataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 39 | feaR_A81P | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagcccataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 40 | feaR_A81S | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagtccataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 41 | feaR_A81V | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggtgataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 42 | feaR_A81E | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggagataatggagcaggatgagcgtcaggtgcagattggcgct |
| | | ggcgatattacgttactcgatgcctcacgcccctgttcgctttactggca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 43 | feaR_A81M | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagatgataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 44 | feaR_A81G | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagggcataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 45 | feaR_A81I | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagatcataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 46 | feaR_A81F | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagttcataatggagcaggatgagcgtcaggtgcagattggcgctgg |
| | | cgatattacgttactcgatgcctcacgcccctgttcgctttactggcagg |
| | | agtcttctaaacagatttcattacttttgccacgcactctgctggaacaat |
| | | attttccccatcaaaaacctatctgcgcagaaagactggacgctgact |
| | | tacccatggtgcaactcagtcatcgcctgttacaggagagcatgaat |
| | | aatccggcactttctgaaacagaaagtgaagctgcgctacaggcga |
| | | tggtgtgtctgctgcgcccggtacttcatcagcgggaatctgttcaacct |
| | | cgtcgtgaacgtcagtttcaaaaagtggttacgttgatagacgataat |
| | | attcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 47 | feaR_A81Y | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagtacataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 48 | feaR_A81W | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagtggataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 49 | feaR_A81C | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagtgcataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 50 | feaR_A81N | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagaacataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 51 | feaR_A81Q | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagcagataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 52 | feaR_A81K | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagaagataatggagcaggatgagcgtcaggtgcagattggcgct |
| | | ggcgatattacgttactcgatgcctcacgcccctgttcgctttactggca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 53 | feaR_A81H | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagcacataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 54 | feaR_A81R | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | cagcgcataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 55 | feaR_A81D | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggacataatggagcaggatgagcgtcaggtgcagattggcgctg |
| | | gcgatattacgttactcgatgcctcacgcccctgttcgctttactggcag |
| | | gagtcttctaaacagatttcattacttttgccacgcactctgctggaaca |
| | | atattttccccatcaaaaacctatctgcgcagaaagactggacgctga |
| | | cttacccatggtgcaactcagtcatcgcctgttacaggagagcatgaa |
| | | taatccggcactttctgaaacagaaagtgaagctgcgctacaggcg |
| | | atggtgtgtctgctgcgcccggtacttcatcagcgggaatctgttcaac |
| | | ctcgtcgtgaacgtcagtttcaaaaagtggttacgttgatagacgataa |
| | | tattcgcgaagagatattacgcccggagtggatagccggagagaca |
| | | ggtatgtcagtacgtagtttgtaccgaatgtttgccgataaaggtttggt |
| | | agtcgcgcaatatattcgtaaccgtcgtctcgatttttgtgcagatgcga |
| | | ttcgccatgccgcagatgatgaaaaactggcaggcatcggctttcatt |
| | | ggggattttctgaccagagtcatttttcaacggtatttaagcaacgctttg |
| | | ggatgacgccaggcgagtatcgacgtaaattccgctaa |
| 56 | feaR_M83G | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataGGCgagcaggatgagcgtcaggtgcagattggcg |
| | | ctggcgatattacgttactcgatgcctcacgcccctgttcgctttactgg |
| | | caggagtcttctaaacagatttcattacttttgccacgcactctgctgga |
| | | acaatattttccccatcaaaaacctatctgcgcagaaagactggacg |
| | | ctgacttacccatggtgcaactcagtcatcgcctgttacaggagagca |
| | | tgaataatccggcactttctgaaacagaaagtgaagctgcgctacag |
| | | gcgatggtgtgtctgctgcgcccggtacttcatcagcgggaatctgttc |
| | | aacctcgtcgtgaacgtcagtttcaaaaagtggttacgttgatagacg |
| | | ataatattcgcgaagagatattacgcccggagtggatagccggaga |
| | | gacaggtatgtcagtacgtagtttgtaccgaatgtttgccgataaaggt |
| | | ttggtagtcgcgcaatatattcgtaaccgtcgtctcgatttttgtgcagat |
| | | gcgattcgccatgccgcagatgatgaaaaactggcaggcatcggct |
| | | ttcattggggattttctgaccagagtcatttttcaacggtatttaagcaac |
| | | gctttgggatgacgccaggcgagtatcgacgtaaattccgctaa |
| 57 | feaR_M83A | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataGCGgagcaggatgagcgtcaggtgcagattggcg |
| | | ctggcgatattacgttactcgatgcctcacgcccctgttcgctttactgg |
| | | caggagtcttctaaacagatttcattacttttgccacgcactctgctgga |
| | | acaatattttccccatcaaaaacctatctgcgcagaaagactggacg |
| | | ctgacttacccatggtgcaactcagtcatcgcctgttacaggagagca |
| | | tgaataatccggcactttctgaaacagaaagtgaagctgcgctacag |
| | | gcgatggtgtgtctgctgcgcccggtacttcatcagcgggaatctgttc |
| | | aacctcgtcgtgaacgtcagtttcaaaaagtggttacgttgatagacg |
| | | ataatattcgcgaagagatattacgcccggagtggatagccggaga |
| | | gacaggtatgtcagtacgtagtttgtaccgaatgtttgccgataaaggt |
| | | ttggtagtcgcgcaatatattcgtaaccgtcgtctcgatttttgtgcagat |
| | | gcgattcgccatgccgcagatgatgaaaaactggcaggcatcggct |
| | | ttcattggggattttctgaccagagtcatttttcaacggtatttaagcaac |
| | | gctttgggatgacgccaggcgagtatcgacgtaaattccgctaa |
| 58 | feaR_M83P | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataCCCgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 59 | feaR_M83V | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataGTTgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 60 | feaR_M83I | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataATCgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 61 | feaR_M83L | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataTTGgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 62 | feaR_M83F | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataTTCgagcaggatgagcgtcaggtgcagattggcgct |
| | | ggcgatattacgttactcgatgcctcacgcccctgttcgctttactggca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 63 | feaR_M83Y | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataTACgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 64 | feaR_M83W | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataTGGgagcaggatgagcgtcaggtgcagattggcgc |
| | | tggcgatattacgttactcgatgcctcacgcccctgttcgTGGtactg |
| | | gcaggagtcttctaaacagatttcattacttttgccacgcactctgctgg |
| | | aacaatattttccccatcaaaaacctatctgcgcagaaagactggac |
| | | gctgacttacccatggtgcaactcagtcatcgcctgttacaggagagc |
| | | atgaataatccggcactttctgaaacagaaagtgaagctgcgctaca |
| | | ggcgatggtgtgtctgctgcgcccggtacttcatcagcgggaatctgtt |
| | | caacctcgtcgtgaacgtcagtttcaaaaagtggttacgttgatagac |
| | | gataatattcgcgaagagatattacgcccggagtggatagccggag |
| | | agacaggtatgtcagtacgtagtttgtaccgaatgtttgccgataaag |
| | | gtttggtagtcgcgcaatatattcgtaaccgtcgtctcgatttttgtgcag |
| | | atgcgattcgccatgccgcagatgatgaaaaactggcaggcatcgg |
| | | ctttcattggggattttctgaccagagtcatttttcaacggtatttaagcaa |
| | | cgctttgggatgacgccaggcgagtatcgacgtaaattccgctaa |
| 65 | feaR_M83S | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataTCCgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 66 | feaR_M83C | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataTGCgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 67 | feaR_M83T | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataACGgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 68 | feaR_M83N | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataAACgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 69 | feaR_M83Q | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataCAAgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 70 | feaR_M83H | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataCACgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 71 | feaR_M83K | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataAAGgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 72 | feaR_M83R | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataCGCgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 73 | feaR_M83D | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataGATgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 74 | feaR_M83E | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataGAGgagcaggatgagcgtcaggtgcagattggcgc |
| | | tggcgatattacgttactcgatgcctcacgcccctgttcgctttactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 75 | feaR_L108G | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgGGGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 76 | feaR_L108A | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgGCGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 77 | feaR_L108P | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgGCGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 78 | feaR_L108V | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgGTCtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 79 | feaR_L108M | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgACGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 80 | feaR_L108I | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgATCtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 81 | feaR_L108F | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgTTTtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 82 | feaR_L108Y | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgTACtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 83 | feaR_L108W | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgTGGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 84 | feaR_L108S | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgTCCtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 85 | feaR_L108C | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgTGCtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 86 | feaR_L108T | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgACGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 87 | feaR_L108N | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgAACtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 88 | feaR_L108Q | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgCAGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 89 | feaR_L108H | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgCACtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 90 | feaR_L108K | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgAAAtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 91 | feaR_L108R | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgCGTtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 92 | feaR_L108D | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgGACtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 93 | feaR_L108E | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgGAGtactggc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 94 | feaR_W110G | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacGGCc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 95 | feaR_W110A | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacGCGc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 96 | feaR_W110P | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacCCGc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 97 | feaR_W110V | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacGTGc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 98 | feaR_W110M | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacATGca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 99 | feaR_W110I | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacATAca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 100 | feaR_W110L | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacCTCca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 101 | feaR_W110F | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacTTCca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 102 | feaR_W110Y | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacTACca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 103 | feaR_W110S | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacTCCca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 104 | feaR_W110C | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacTGTca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 105 | feaR_W110T | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacACCca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 106 | feaR_W110N | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacAACca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 107 | feaR_W110Q | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacCAGc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 108 | feaR_W110H | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacCACca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 109 | feaR_W110K | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacAAGca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 110 | feaR_W110R | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacCGCc |
| | | aggagtcttctaaacagatttcattacttttgccacgcactctgctggaa |
| | | caatattttccccatcaaaaacctatctgcgcagaaagactggacgct |
| | | gacttacccatggtgcaactcagtcatcgcctgttacaggagagcatg |
| | | aataatccggcactttctgaaacagaaagtgaagctgcgctacagg |
| | | cgatggtgtgtctgctgcgcccggtacttcatcagcgggaatctgttca |
| | | acctcgtcgtgaacgtcagtttcaaaaagtggttacgttgatagacgat |
| | | aatattcgcgaagagatattacgcccggagtggatagccggagaga |
| | | caggtatgtcagtacgtagtttgtaccgaatgtttgccgataaaggtttg |
| | | gtagtcgcgcaatatattcgtaaccgtcgtctcgatttttgtgcagatgc |
| | | gattcgccatgccgcagatgatgaaaaactggcaggcatcggctttc |
| | | attggggattttctgaccagagtcatttttcaacggtatttaagcaacgct |
| | | ttgggatgacgccaggcgagtatcgacgtaaattccgctaa |
| 111 | feaR_W110D | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacGATca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| 112 | feaR_W110E | ATGaaccccgcagtggataatgagtttcagcaatggctttcccaaat |
| | | caatcaggtatgcggaaattttaccggacgcctgctgactgagcgtta |
| | | cacgggtgtactggacacgcattttgccaaaggactaaagctgagta |
| | | ccgtgacaaccagcggggtgaatttatcccgcacctggcaggaagt |
| | | aaaaggcagcgacgatgcctggttttacaccgtttttcagcttagtggt |
| | | caggcaataatggagcaggatgagcgtcaggtgcagattggcgctg |
| | | gcgatattacgttactcgatgcctcacgcccctgttcgctttacGAAca |
| | | ggagtcttctaaacagatttcattacttttgccacgcactctgctggaac |
| | | aatattttccccatcaaaaacctatctgcgcagaaagactggacgctg |
| | | acttacccatggtgcaactcagtcatcgcctgttacaggagagcatga |
| | | ataatccggcactttctgaaacagaaagtgaagctgcgctacaggc |
| | | gatggtgtgtctgctgcgcccggtacttcatcagcgggaatctgttcaa |
| | | cctcgtcgtgaacgtcagtttcaaaaagtggttacgttgatagacgata |
| | | atattcgcgaagagatattacgcccggagtggatagccggagagac |
| | | aggtatgtcagtacgtagtttgtaccgaatgtttgccgataaaggtttgg |
| | | tagtcgcgcaatatattcgtaaccgtcgtctcgatttttgtgcagatgcg |
| | | attcgccatgccgcagatgatgaaaaactggcaggcatcggctttca |
| | | ttggggattttctgaccagagtcatttttcaacggtatttaagcaacgcttt |
| | | gggatgacgccaggcgagtatcgacgtaaattccgctaa |
| | TrpR sensor | |
| 113 | trpR (WT) | atggcccaacaatcaccctattcagcagcgatggcagaacagcgtc |
| | | accaggagtggttacgttttgtcgacctgcttaagaatgcctaccaaa |
| | | acgatctccatttaccgttgttaaacctgatgctgacgccagatgagcg |
| | | cgaagcgttggggactcgcgtgcgtattgtcgaagagctgttgcgcg |
| | | gcgaaatgagccagcgtgagttaaaaaatgaactcggcgcaggca |
| | | tcgcgacgattacgcgtggatctaacagcctgaaagccgcgcccgt |
| | | cgagctgcgccagtggctggaagaggtgttgctgaaaagcgattga |
| 114 | trpR (OD) | atggcccaacaatcaccctattcagcagcgatggcagaacagcgtc |
| | | accaggagtggttacgttttgtcgacctgcttaagaatgcctaccaaa |
| | | acgatctccatttaccgttgttaaacctgatgctgacgccagatgagcg |
| | | cgaagcgttggggactcgcgtgcgtattgtcgaagagctgttgcgcg |
| | | gcgaaatgagccagcgtgagttaagcaatgaactcggcgcatgctg |
| | | cctgacgattcgccgtggatctaacagcctgaaagccgcgcccgtc |
| | | gagctgcgccagtggctggaagaggtgttgctgaaaagcgattga |
| 115 | trpR (O1) | atggcccaacaatcaccctattcagcagcgatggcagaacagcgtc |
| | | accaggagtggttacgttttgtcgacctgcttaagaatgcctaccaaa |
| | | acgatctccatttaccgttgttaaacctgatgctgacgccagatgagcg |
| | | cgaagcgttggggactcgcgtgcgtattgtcgaagagctgttgcgcg |
| | | gcgaaatgagccagcgtgagttaagcaatgaactcggcgcaaact |
| | | ggcgcacgattaagcgtggatctaacagcctgaaagccgcgcccgt |
| | | cgagctgcgccagtggctggaagaggtgttgctgaaaagcgattga |
| 116 | trpR with RBS | aaccgggggaggcattttgcttcccccgctaacaatggcgacatatt |
| | | ATGgcccaacaatcaccctattcagcagcgatggcagaacagcg |
| | | tcaccaggagtggttacgttttgtcgacctgcttaagaatgcctaccaa |
| | | aacgatctccatttaccgttgttaaacctgatgctgacgccagat |
| | | gagcgcgaagcgttggggactcgcgtgcgtattgtcgaagagctgtt |
| | | gcgcggcgaaatgagccagcgtgagttaaaaaatgaactcggcgc |
| | | aggcatcgcgacgattacgcgtggatctaacagcctgaaagccgcg |
| | | cccgtcgagctgcgccagtggctggaagaggtgttgctgaa |
| | | aagcgattga |
| 117 | trpR (OD) with | aaccgggggaggcattttgcttcccctgctaacaaaggagacattttA |
| | RBS | TGgcccaacaatcaccctattcagcagcgatggcagaacagcgtc |
| | | accaggagtggttacgttttgtcgacctgcttaagaatgcctaccaaa |
| | | acgatctccatttaccgttgttaaacctgatgctgacgccagat |
| | | gagcgcgaagcgttggggactcgcgtgcgtattgtcgaagagctgtt |
| | | gcgcggcgaaatgagccagcgtgagttaaaaaatgaactcggcgc |
| | | aggcatcgcgacgattacgcgtggatctaacagcctgaaagccgcg |
| | | cccgtcgagctgcgccagtggctggaagaggtgttgctgaa |
| | | aagcgattga |
| 118 | trpR (O1) with | aaccgggggaggcattttgcttcccccgctaacaatggcgacatatt |
| | RBS | ATGgcccaacaatcaccctattcagcagcgatggcagaacagcg |
| | | tcaccaggagtggttacgttttgtcgacctgcttaagaatgcctaccaa |
| | | aacgatctccatttaccgttgttaaacctgatgctgacgccagat |
| | | gagcgcgaagcgttggggactcgcgtgcgtattgtcgaagagctgtt |
| | | gcgcggcgaaatgagccagcgtgagttaagcaatgaactcggcgc |
| | | aaactggcgcacgattaagcgtggatctaacagcctgaaagccgc |
| | | gcccgtcgagctgcgccagtggctggaagaggtgttgctgaa |
| | | aagcgattga |
| 119 | Ptrp | gagctgttgacaattaatcatcgaactagttaactagtacgc |
| 120 | Ptrp(O_{D}) | gagctgttgacaattaatcatcgagtaagttaacttacacgc |
| 121 | Ptrp(O₁) | gagctgttgacaattaatcatcgatgtagttaactacaacgc |
| | Reference (WT) | |
| | Amino Acid | |
| | Sequences | |
| 122 | trpR (WT) | MAQQSPYSAAMAEQRHQEWLRFVDLLKNAYQN |
| | | DLHLPLLNLMLTPDEREALGTRVRIVEE |
| | | LLRGEMSQRELKNELGAGIATITRGSNSLKAAPVE |
| | | LRQWLEEVLLKSD |
| 123 | tynA (WT) | MGSPSLYSARKTTLALAVALSFAWQAPVFAHGG |
| | | EAHMVPMDKTLKEFGADVQWDDYAQLF |
| | | TLIKDGAYVKVKPGAQTAIVNGQPLALQVPVVMK |
| | | DNKAWVSDTFINDVFQSGLDQTFQVE |
| | | KRPHPLNALTADEIKQAVEIVKASADFKPNTRFTEI |
| | | SLLPPDKEAVWAFALENKPVDQPR |
| | | KADVIMLDGKHIIEAVVDLQNNKLLSWQPIKDAHG |
| | | MVLLDDFASVQNIINNSEEFAAAVK |
| | | KRGITDAKKVITTPLTVGYFDGKDGLKQDARLLKV |
| | | ISYLDVGDGNYWAHPIENLVAVVDL |
| | | EQKKIVKIEEGPVVPVPMTARPFDGRDRVAPAVK |
| | | PMQIIEPEGKNYTITGDMIHWRNWDF |
| | | HLSMNSRVGPMISTVTYNDNGTKRKVMYEGSLG |
| | | GMIVPYGDPDIGWYFKAYLDSGDYGMG |
| | | TLTSPIARGKDAPSNAVLLNETIADYTGVPMEIPR |
| | | AIAVFERYAGPEYKHQEMGQPNVST |
| | | ERRELVVRWISTVGNYDYIFDWIFHENGTIGIDAG |
| | | ATGIEAVKGVKAKTMHDETAKDDTR |
| | | YGTLIDHNIVGTTHQHIYNFRLDLDVDGENNSLVA |
| | | MDPVVKPNTAGGPRTSTMQVNQYNI |
| | | GNEQDAAQKFDPGTIRLLSNPNKENRMGNPVSY |
| | | QIIPYAGGTHPVAKGAQFAPDEWIYHR |
| | | LSFMDKQLWVTRYHPGERFPEGKYPNRSTHDTG |
| | | LGQYSKDNESLDNTDAVVWMTTGTTHV |
| | | ARAEEWPIMPTEWVHTLLKPWNFFDETPTLGALK |
| | | KDK |
| 124 | tyrR (WT) | MRLEVFCEDRLGLTRELLDLLVLRGIDLRGIEIDPI |
| | | GRIYLNFAELEFESFSSLMAEIRR |
| | | IAGVTDVRTVPWMPSEREHLALSALLEALPEPVL |
| | | SVDMKSKVDMANPASCQLFGQKLDRL |
| | | RNHTAAQLINGFNFLRWLESEPQDSHNEHVVING |
| | | QNFLMEITPVYLQDENDQHVLTGAVV |
| | | MLRSTIRMGRQLQNVAAQDVSAFSQIVAVSPKMK |
| | | HVVEQAQKLAMLSAPLLITGDTGTGK |
| | | DLFAYACHQASPRAGKPYLALNCASIPEDAVESE |
| | | LFGHAPEGKKGFFEQANGGSVLLDEI |
| | | GEMSPRMQAKLLRFLNDGTFRRVGEDHEVHVDV |
| | | RVICATQKNLVELVQKGMFREDLYYRL |
| | | NVLTLNLPPLRDCPQDIMPLTELFVARFADEQGV |
| | | PRPKLAADLNTVLTRYAWPGNVRQLK |
| | | NAIYRALTQLDGYELRPQDILLPDYDAATVAVGED |
| | | AMEGSLDEITSRFERSVLTQLYRNY |
| | | PSTRKLAKRLGVSHTAIANKLREYGLSQKKNEE |
| 125 | feaR (WT) | MNPAVDNEFQQWLSQINQVCGNFTGRLLTERYT |
| | | GVLETHFAKGLKLSTVTTNGVNLYRTW |
| | | QEIKGSDDAWFYTVFQLSGQAIMEQDERQVQIGA |
| | | GDITLLDASRPCSLYWQESSKQISLL |
| | | LPRTLLEQYFPHQKPVCAERLDADLPMVQLSHRL |
| | | LQESMNNPALSETESEAALQAMVCLL |
| | | RPVLHQRESVQPRRERQFQKVVTLIDDNIREEILR |
| | | PEWIAGETGMSVRSLYRMFADKGLV |
| | | VAQYIRNRRLDFCADAIRHAADDEKLAGIGFHWG |
| | | FSDQSHFSTVFKQRFGMTPGEYRRKF |
| | | R |

All plasmids were assembled in E. coli DH10B using the Gibson Assembly or Golden Gate Assembly methods. EcN was transformed with the purified and sequence-verified plasmids for testing. The EcN strain used in this work lacks its native plasmids (obtained from DSMZ). The tynA, feaR, and tyrR genes and PtynA, PtyrP, PtyrR, Pmtr, PtyrB, ParoF, and ParoP promoters were obtained from E. coli MG1655 genomic DNA. pHJY23 containing the inactivated tynA gene (see e.g., FIG. 3C) was constructed by removing the internal region (973-2230th bases) of tynA from plasmid pHJY22 by digesting with Eael and ligating the linear product. The pHJY028 PtynA reporter plasmid was optimized by randomizing the ribosome binding site for GFP and assaying variants for a response to DA.

Plasmid DNA was isolated using the PureLink Quick Plasmid Miniprep Kit (Invitrogen, Walthem, Mass., USA), and PCR products were extracted from electrophoresis gels using the Zymoclean Gel DNA Recovery Kit (ZYMO research, Irvine, Calif., USA). Enzymes were purchased from New England Biolabs (Ipswich, Mass., USA). Chemicals were purchased from Sigma-Aldrich (St. Louis, Mo., USA) or Gold Biotechnology (Olivette, Mo., USA). All sequencing was performed by Genewiz (South Plainfield, N.J., USA). Primers were purchased from Integrated DNA Technologies (Coralville, Iowa, USA).

Aromatic Amino Acid Sensing Assay

Everything but (Eb) medium was prepared by supplementing M9 minimal medium with 1 mM MgSO4, 100 mM CaCl2), 0.4% w/v glucose, and all non-aromatic amino acids (0.8 mM alanine, 5 mM arginine, 0.4 mM asparagine, 0.4 mM aspartate, 0.1 mM cysteine, 0.6 mM glutamate, 0.6 mM glutamine, 0.8 mM glycine, 0.2 mM histidine, 0.4 mM isoleucine, 0.8 mM leucine, 0.4 mM lysine, 0.2 mM methionine, 0.4 mM proline, 10 mM serine, 0.4 mM threonine, and 0.6 mM valine). Single colonies of EcN containing the relevant sensor plasmids were transferred to 5 mL Eb medium in 14 mL round bottom tubes and incubated overnight at 250 rpm and 37° C. Experimental cultures were prepared by diluting overnight cultures 200× into 0.6 mL fresh Eb medium supplemented with the respective ligands (Phe and Tyr) in 2 mL 96-deep well plates (Eppendorf, Hamburg, Germany). Cultures were grown for 8 h at 37° C. and 250 rpm before sampled for fluorimetry or flow cytometry analysis. All medium was supplemented with the relevant antibiotics for plasmid maintenance (34 mg/ml chloramphenicol and 100 mg/ml spectinomycin).

Aromatic Amine Sensing Assays

For fluorescence-based quantification, single colonies were transferred to 5 mL LB medium (VWR, Radnor, Pa., USA) in 14 mL round bottom tubes and incubated overnight at 250 rpm and 37° C. Unless otherwise specified, experimental cultures were prepared by diluting overnight cultures 100× into 0.6 mL M9 minimal medium supplemented with 1 mM MgSO4, 100 mM CaCl2), and 2% w/v glycerol as well as the respective ligands (DA, PEA, Tyra, and Trypta) in 2 mL 96-deep well plates. Cultures were grown for 24 h at 37° C. and 250 rpm. After 5 h and 24 h of incubation, samples were obtained from the cultures for flow cytometry analysis.

For growth-based library screening, b/a (encoding b-lactamase) and sacB (encoding levansucrase) were incorporated downstream of gfp in the pHJY028 reporter plasmid (see e.g., TABLE 4). For optimization, ribosome binding site libraries were designed for b/a and sacB using the RBS Calculator (see e.g., TABLE 6). The optimization resulted in reporter pCX008. To test sensor variants using the pCX008 reporter, single colonies were transferred to 0.6 mL LB medium in 2 mL 96-deep well plates and incubated overnight at 250 rpm and 37° C. The overnight cultures were diluted 50× into 0.6 mL M9 minimal medium supplemented with 1 mM MgSO4, 100 mM CaCl2, and 2% w/v glycerol in 2 mL 96-well deep well plates. For positive selection, cultures were supplemented with 300 mg/mL carbenicillin. Cultures were then incubated for 2 h at 250 rpm and 37° C. After the 2 h incubation, 2 mL of cells were plated onto M9 agar plates supplemented with 2% glycerol and either 300 mg/mL carbenicillin and 1 mM of the amine of interest (positive selection) or 5% (w/v) sucrose and 1 mM of the non-desired amines (negative selection). Plates were incubated overnight at 37° C.

Fluorimetry

200 mL culture samples were collected and transferred to 96-well assay microplates (clear, flat bottom black, Greiner Bio-One). The fluorescence and culture absorbance (Abs) were measured using a Tecan microplate reader (Infinite M200 Pro) as previously described. The fluorescence of GFP was measured with an excitation at 483 nm and emission at 530 nm. The Abs of the samples was measured at 600 nm. The measured fluorescence was normalized by dividing by the Abs and subtracting the same ratio obtained from non-fluorescent wild-type cells (Equation 1).

$$Fluorescence ( au ) = \frac{{Fluorescence}_{sample}}{{Abs ( 600 nm )}_{sample}} - \frac{{Fluorescence}_{wild - type}}{{Abs ( 600 nm )}_{sample}} \qquad Equation 1$$

Flow Cytometry

Flow cytometry was performed as previously described. Culture samples were collected and diluted to a final OD600 of ˜0.005-0.01 in 200 mL filtered phosphate-buffered saline supplemented with 2 mg/ml kanamycin in 96-well assay microplates (U-bottom, REF-353910 from BD Biosciences, San Jose, Calif., USA). The fluorescence of the samples was measured using a Millipore Guava EasyCyte High Throughput flow cytometer with a 488 nm excitation laser and 512/18 nm emission filter. Cytometry data was gated by forward and side scatter. FlowJo (TreeStar Inc.) was used to obtain the arithmetic mean of the fluorescence distribution. The averages of the arithmetic means were calculated from three biological replicates. The average fluorescence of the non-fluorescent wild-type cell was subtracted from each sample to obtain the final fluorescence (au) values (Equation 2). To obtain the relative fluorescence in FIG. 6A-FIG. 6C, fluorescence values for each variant were normalized to the fluorescence of the wild-type sensor using Equation 3.

$$Fluorescence ( au ) = {Fluorescence}_{sample} - {Fluorescence}_{wild - type} \qquad Equation 2$$

$$Relative Fluorescence = \frac{{Fluroescence}_{Amine variant} - {Fluorescence}_{No amine variant}}{{Fluorescence}_{Amine WT FeaR} - {Fluorscence}_{No amine WT FeaR}} \qquad Equation 3$$

Hill Equation Fitting

The Hill equation (Equations 4 and 5) was used to fit lines to the fluorescence data. The model was fit to the experimentally collected data by minimizing the root mean square error (RMSE; Equation 6). Fitted values are listed in TABLE 2.

For repressible constructs:

$$F = {F}_{max} - \frac{( {F}_{max} - {F}_{min )}}{1 + {( \frac{{K}_{A}}{[ L ]} )}^{n}} \qquad Equation 4$$

For inducible constructs:

$$F = {F}_{min} + \frac{( {F}_{max} - {F}_{min )}}{1 + {( \frac{{K}_{A}}{[ L ]} )}^{n}} \qquad Equation 5$$

where

F=Calculated fluorescence

Fₘₐₓ=Maximum fluorescence

Fₘᵢₙ=Minimum fluorescence

K_{A}=Half maximal constant

n=Hill coefficient

[L]=Ligand concentration

$$RMSE = \sqrt{{\sum }_{N = 1}^{N}} \frac{{( F - {F}_{exp} )}^{2}}{N} \qquad Equation 6$$

where

F=Calculated fluorescence

Fₑₓₚ=Actual experimental fluorescence

N=Number of data points

FeaR Protein Modeling

FeaR structural motifs were annotated using the MOTIF Search web tool. Structural predictions were made using the comparative modeling function of the Robetta web server with the wild-type or mutant FeaR amino acid sequences as inputs. Predicted structures were used as the basis for ligand docking simulations. Three-dimensional conformers of the aldehydes of DA, PEA, Tyra, and Trypta were generated using Chem3D 16.0 (PerkinElmer, Waltham, Mass.). Ligand conformer and protein structure.pdb files were uploaded to the Rosetta Ligand Docking Server hosted by ROSIE, with the ligand of interest initially centered at the coordinates of the 81st residue's side chain. All protein structures were visualized using PyMOL 2.4.1 (Schrodinger, Inc., New York, N.Y.). Values for amino acid size and hydropathy index were obtained from literature for the analysis in FIG. 5D and are shown in TABLE 3.

Quantification and Statistical Analysis

All statistical details of experiments, including significance criteria and sample size can be found in the figure legends. No sample size calculations were performed during the design of experiments. No samples were excluded.

### Example 2: Engineering of a Tryptamine Biosensor

This example describes design and development of a tryptamine biosensor.

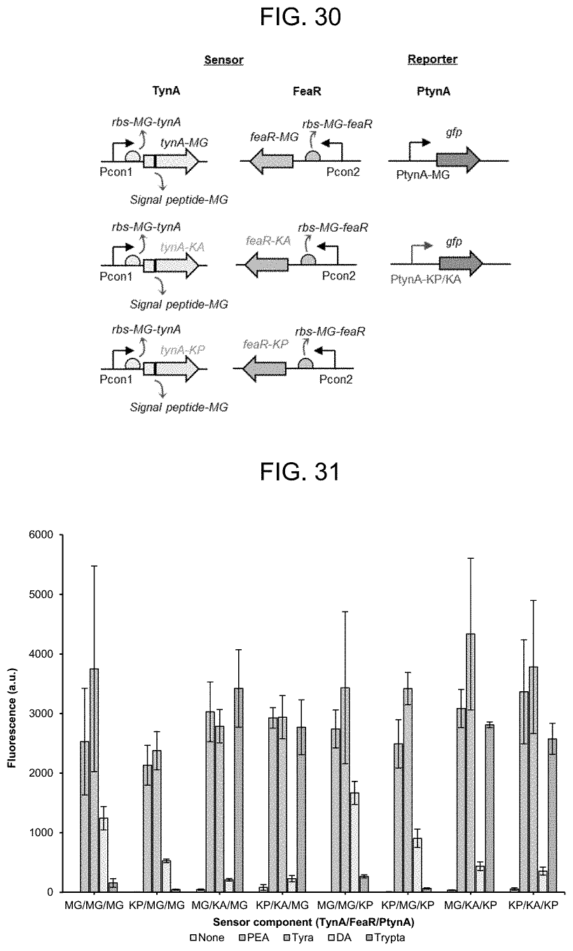

Tryptamine specific biosensors were first designed by genetic parts swapping. Three types of tynA and feaR genes from E. coli K-12 MG1655 (MG), Klebsiella aerogenes (KA), and Klebsiella pneumoniae (KP), and two types of PtynA sequences were chosen and tested in 18 different combinations (see e.g., FIG. 30). PtynA-KP/KA was constructed by replacing the FeaR binding sites of PtynA-MG with those from Klebsiella species. Sequences of those constructs are shown in TABLE 7.

TABLE 7
Genetic constructs used in this work.

| SEQ ID | Recombinant | | |
|---|---|---|---|
| NO: | DNA | Sequence | Source |
| 126 | tynA-KA | atggcaaacggcttgatgttttcccctcgtaaaaccgc | Klebsiella |
| | | gctggcgctggctgtcgcggtggtttgcgcctggcaat | aerogenes |
| | | caccggtcttcgctcacggtagcgaagcgcacatgg | ATCC15038 |
| | | tgccgttggataaaacgctgcaggcgttcggcgccg | genome |
| | | atgtgcagtgggatgactacgcgcaaatgttcaccct | |
| | | gataaaagacggcgcttacgtcaaagtaaaacccg | |
| | | gcgccaaaacggcgatcgtcaacggtaaacccctt | |
| | | gatctacaggtgccggtagtaatgaaggaaggtaa | |
| | | agcctgggtctccgatacctttatcaacgatgtattcca | |
| | | gtccggtctcgatcagaccttccaggtagaaaaacg | |
| | | ccctcacccgttaaattcgctctcggcggcggaaatc | |
| | | ggtgaagcagtgaccattgttaaagccgcgccgga | |
| | | gttccagccgaatacccgctttactgagatttccttaca | |
| | | cgagccggataaagcggcggtatgggcctttgccct | |
| | | gcagggaacacccgttgatgccccccgcaccgccg | |
| | | atgtggtgatgctcgatggcaaacatgtcattgaagc | |
| | | cgtcgtcgatctgcaaaacaaaaaaatcctctcatgg | |
| | | acgccgattaaaggcgcccacgggatggtcttgcttg | |
| | | atgacttcgtcagcgtgcagaacattatcaatgccag | |
| | | cagcgagttcgctgaggtgctgaaaaagcacggtat | |
| | | taccgaccccagtaaggtggtcaccaccccgctcac | |
| | | cgtcggctactttgatggcaaagatggcctgcagca | |
| | | ggatgcgcgtctgctgaaagtcgtcagttatctggata | |
| | | ccggcgacggcaactactgggcgcacccgattgaa | |
| | | aacctggtggcggtggtcgaccttgaagcgaagaa | |
| | | aatcatcaaaatcgaagaaggcccggtgatcccgg | |
| | | taccgatggagccacgtccgtatgatggtcgcgacc | |
| | | gcaacgccccggcggtgaaaccgctggagataac | |
| | | cgaaccggaaggcaaaaactacacgatcaccggc | |
| | | gataccattcactggcggaactgggattttcatctgcg | |
| | | cctgaactcgcgcgtcgggcccattctctcgacggtg | |
| | | acttacaacgacaacggcacaaaacgccaggtgat | |
| | | gtatgaaggttccctcggcgggatgatcgtcccctac | |
| | | ggcgacccggacgtcggctggtatttcaaagcctatc | |
| | | tggactccggcgattacggcatgggcaccctgacgt | |
| | | cgccgattgttcgcggtaaagatgcgccgtcaaatgc | |
| | | ggtactgctggacgaaactatcgccgattacaccgg | |
| | | caaaccgaccactattccaggcgcggtcgccatatt | |
| | | cgaacgctatgccgggcctgagtataagcacctgga | |
| | | aatgggcaaacccaacgtcagcaccgaacgccgg | |
| | | gaactggtggtgcgctggatcagtaccgtcgggaac | |
| | | tatgattatatctttgactgggtgttccatgacaacggc | |
| | | accatcggtatcgatgccggcgccaccggcattgaa | |
| | | gccgttaaaggcgtgaaggcgaagaccatgcacg | |
| | | accccagcgccaaagaggatacccgctacgggac | |
| | | gctaatcgaccataatattgtcggcaccacccacca | |
| | | gcatatttataatttccgectcgatctcgacgtggacgg | |
| | | cgaaaacaacaccctggtggcgatggatcctgaag | |
| | | tgaagccaaacaccgccggcggcccgcgcaccag | |
| | | caccatgcaggtgaatcagtacacaatcgatagcg | |
| | | agcagaaagcggcgcagaaattcgaccctggcact | |
| | | atccgcctgctgagcaacaccagcaaagagaacc | |
| | | gcatgggtaacccggtctcttaccagattatcccttatg | |
| | | ccggcggcacgcatccggcggcgaccggcgccaa | |
| | | gttcgccccggacgagtggatatatcatcgcctgagc | |
| | | tttatggataaacagctgtgggtgacgcgttaccacc | |
| | | cgacagagcgttatccggaagggaaatatcccaac | |
| | | cgttccgcccaggataccggcttaggccagtacgcg | |
| | | aaggatgatgagtcgctgactaaccacgacgacgt | |
| | | cgtgtggatcaccaccggcaccacccacgtcgcgc | |
| | | gcgccgaagagtggccaattatgccgaccgagtgg | |
| | | gcgcatgcgctgctcaaaccgtggaacttctttgacg | |
| | | aaaccccaacgcttggcgagaagaaaaagtaa | |
| 127 | tynA-KP | ATGGCAAATGGACTGAAATTCAGCC | IDT |
| | | CTCGTAAAACAGCTCTTGCCCTGGC | |
| | | CGTTGCCGTCGTTTGTGCTTGGCAA | |
| | | TCCCCTGTGTTTGCTCACGGTTCCG | |
| | | AGGCACATATGGTACCCTTGGACAA | |
| | | AACATTACAGGAGTTTGGGGCCGAC | |
| | | GTTCAGTGGGACGACTACGCACAGA | |
| | | TGTTTACTCTTATCAAGGACGGAGCT | |
| | | TATGTGAAAGTCAAGCCTGGAGCAA | |
| | | AAACTGCAATTGTAAATGGAAAGTCT | |
| | | TTAGATCTTCCCGTCCCGGTTGTGAT | |
| | | GAAGGAAGGCAAAGCCTGGGTTAGC | |
| | | GACACTTTTATCAATGATGTGTTTCA | |
| | | ATCCGGCCTTGATCAGACCTTTCAG | |
| | | GTGGAAAAGCGTCCTCATCCCTTGA | |
| | | ATAGTTTGAGTGCAGCCGAGATCTC | |
| | | TGAGGCTGTTACAATCGTTAAGGCA | |
| | | GCTCCGGAATTTCAACCAAATACCC | |
| | | GCTTTACAGAGATTTCATTGCACGAA | |
| | | CCGGACAAGGCTGCGGTCTGGGCT | |
| | | TTTGCCCTGCAAGGTACGCCAGTTG | |
| | | ATGCACCGCGTACCGCGGACGTCGT | |
| | | CATGTTAGACGGTAAGCATGTTATC | |
| | | GAAGCAGTAGTAGACTTGCAGAATA | |
| | | AAAAAATTCTTTCGTGGACTCCGATT | |
| | | AAGGGGGCCCACGGTATGGTCCTGT | |
| | | TAGACGATTTCGTCTCCGTACAAAAC | |
| | | ATTATCAATGCCTCATCCGAGTTTGC | |
| | | AGAGGTCTTGAAAAAACACGGAATT | |
| | | ACTGACCCGGGGAAAGTCGTGACAA | |
| | | CTCCCCTGACTGTAGGTTTTTTTGAT | |
| | | GGCAAGGATGGATTGCAACAAGACG | |
| | | CACGCCTGTTAAAAGTCGTCTCGTAT | |
| | | CTGGATACGGGTGATGGTAATTACT | |
| | | GGGCGCACCCTATCGAGAACTTAGT | |
| | | GGCCGTAGTTGACCTGGAGGCCAA | |
| | | GAAAATTATCAAAATCGAAGAAGGTC | |
| | | CAGTGATTCCCGTACCGATGGAGCC | |
| | | CCGTCCATACGATGGGCGCGACCG | |
| | | CAATGCCCCAGCTGTCAAACCTCTG | |
| | | GAAATCACAGAGCCTGAAGGGAAAA | |
| | | ATTATACTATTACTGGTGATACTATC | |
| | | CACTGGCAGAATTGGGACTTCCATC | |
| | | TGCGTCTTAATAGTCGCGTGGGACC | |
| | | CATTTTATCAACAGTCACTTACAATG | |
| | | ACAATGGGACAAAGCGTCAGGTGAT | |
| | | GTATGAGGGTAGTCTTGGGGGGATG | |
| | | ATTGTCCCCTACGGTGATCCTGATG | |
| | | TAGGGTGGTATTTCAAAGCTTATCTT | |
| | | GACTCCGGCGATTACGGCATGGGAA | |
| | | CGCTTACGTCGCCTATTGTTCGTGG | |
| | | CAAAGATGCTCCCTCGAATGCCGTT | |
| | | CTTTTGGATGAGACGATCGCTGATTA | |
| | | CACAGGCAAGCCTACTACGATTCCG | |
| | | GGGGCGGTGGCGATTTTTGAACGTT | |
| | | ACGCAGGACCTGAGTACAAACACCT | |
| | | TGAGATGGGAAAGCCCAATGTCAGC | |
| | | ACTGAACGCCGTGAGCTTGTCGTAC | |
| | | GTTGGATCTCAACCGTTGGTAATTAT | |
| | | GACTATATTTTCGACTGGGTATTTCA | |
| | | CGACAATGGAACGATTGGCATCGAT | |
| | | GCTGGGGCTACAGGGATCGAGGCA | |
| | | GTGAAAGGAGTGTTGGCAAAAACTA | |
| | | TGCATGACCCTTCGGCTAAAGAGGA | |
| | | TACACGCTACGGCACTCTGATTGAC | |
| | | CACAACATTGTGGGTACAACGCATC | |
| | | AGCACATCTATAACTTTCGTTTGGAC | |
| | | CTTGACGTTGATGGGGAGAATAACA | |
| | | CGCTTGTCGCGATGGACCCGGAAGT | |
| | | TAAGCCGAATACTGCGGGTGGTCCG | |
| | | CGCACGTCAACTATGCAAGTTAACC | |
| | | AGTACACGATCGACAGCGAGCAAAA | |
| | | GGCGGCTCAAAAATTCGATCCAGGA | |
| | | ACAATTCGTCTTTTATCTAATACCTC | |
| | | GAAGGAAAACCGTATGGGCAACCCG | |
| | | GTGTCCTACCAGATCATCCCATATG | |
| | | CAGGCGGTACTCACCCTGCCGCTAC | |
| | | CGGAGCCAAGTTTGCGCCCGACGA | |
| | | GTGGATTTATCACCGCCTGTCCTTTA | |
| | | TGGACAAACAATTATGGGTAACCCG | |
| | | TTACCACCCGACTGAACGCTATCCT | |
| | | GAAGGTAAGTACCCAAATCGCAGCG | |
| | | CACATGATACTGGATTAGGGCAATAT | |
| | | GCTAAGGATGATGAGTCATTGACTA | |
| | | ACCACGATGACGTTGTGTGGATCAC | |
| | | GACAGGAACCACTCATGTAGCGCGT | |
| | | GCAGAGGAGTGGCCTATTATGCCAA | |
| | | CGGAATGGGCGCATGCACTTCTGAA | |
| | | ACCTTGGAACTTTTTTGATGAGACTC | |
| | | CGACGTTAGGTGAAAAGAAGAAATG | |
| | | A | |
| 128 | feaR-KA | ATGGCGACAGTAGAATCCGGTCAAG | IDT |
| | | ATTATCAGCGCTGGTTGGCTAAGATT | |
| | | AACCAGGTATGCGGGCACTTCGCTG | |
| | | CACGCCCGCTTGGAGACGAATTTCA | |
| | | CGGGGAAATTGATGCGTCGTACACC | |
| | | GGTTCGTTGAAGGTAAGTACGGTCA | |
| | | CAGCACGCGGCGTAAACTTATATCG | |
| | | TACTCGTAACGAGATTAAACGTGACA | |
| | | ACGACGCATGGTTCTATACAGTGTT | |
| | | CCAGTTGGAAGGGCAAGCAGCTATT | |
| | | GAGCAGGATGAGCGCCAGGTCGCG | |
| | | TTAGAGGCAGGAGATATTACATTGAT | |
| | | CGACGCTAGTCGCCCCTGCAGTATT | |
| | | TTCTGGCAACACACATCTAAGCAGG | |
| | | CATCACTTCTTCTTCCGCGCCAATTA | |
| | | GTCGAGAGCCAATTGCATGGAGGAG | |
| | | ACATCTCTATTGCGACACGTTTGAAT | |
| | | AAAACGCTGCCTATGGTCCAATTAA | |
| | | GTCAACGTCTTCTGCAAGAATCTCTT | |
| | | CGCAGTCCTGCATTAAGTGAGTCGG | |
| | | AGGGGGAAGCGGCATTAGAGGCCA | |
| | | TTATCTGTCTTCTGCGCCCCATGCTG | |
| | | CTGCAACAAGACGCTCCGCCTACTC | |
| | | GCCGTGATAAACAGTTCCAAAAAATT | |
| | | ATGGCTTTGATTGACGAATCCATCCA | |
| | | ATCTGAGCAACTTCGCCCAGAATGG | |
| | | CTGGCGGGCGAAACAGGAATGAGT | |
| | | GTTCGTAGTCTGTACCGCCTGTTCG | |
| | | CTGATAAAGGACTTGTGGTTGCTCA | |
| | | GTATATTAAGAATCGCCGCTTGGATC | |
| | | TGTGTGCTCGTGCATTGCAGAATGC | |
| | | ACATGATGAAGAAAAATTGGCAGGG | |
| | | ATCGGGTACTCATGGGGCTTCTCAG | |
| | | ATCACTCGCATTTCAGCACAGCGTT | |
| | | CAAGCAGCGCTTCGGCGTATCCCCA | |
| | | GGCGAGTATCGTAAGCGTTACCGCT | |
| | | GA | |
| 129 | feaR-KP | ATGCCCGAAGCCACTCAAATGCAAC | IDT |
| | | AATGTGGCAATATCGTTATCACTGG | |
| | | GGAAGGTCGTATGATGACAACTGCG | |
| | | GAGGGTGGTTTAGCATATCAACGCT | |
| | | GGTTGGCAACCATTAACCAAGTGTG | |
| | | TGGTCACTTTGCTGCGCGTCCCTTA | |
| | | AAGGAAAGTTTCCACGGTGAAATCG | |
| | | ATGCTCGCTATGCGGGGTCGTTAAA | |
| | | GGTGTCTACCGTTACTGCTGCAGGA | |
| | | GTCAACCTTTATCGCACGCGTAATG | |
| | | AAATCAAACGCGATAACGATGCATG | |
| | | GTTCTATACTGTCTTCCAGTTGGCTG | |
| | | GCGAAGCGATCATCGAACAGGATGA | |
| | | TCGCCAAGTGACGCTGGCAGCAGG | |
| | | GGATATTACCCTTATTGACGCCGCC | |
| | | CGCCCATGTAGCATTGTATGGCAGC | |
| | | AAACATCGCGTCAAGCGTCACTTTTA | |
| | | TTACCCCGCCAACGTGTAGCACCGA | |
| | | CAGGTGATATTACAACAGCGTGTCG | |
| | | CTTGGACAAGAGCCTTCCTATGGTA | |
| | | CAACTGTCACAACGTCTTTTGTTAGA | |
| | | GTCGATGGGCGGAACGACTCTGTCC | |
| | | GCGTCAGAGTCTGAGGCGGCTCTG | |
| | | GAAGCAATCGCCTGCTTATTACGCC | |
| | | CCGTGCTTCATCAGCGTGATCCAGC | |
| | | GCCGAGTCGCCGTGTCAAACAGTTT | |
| | | CAGAAGATCATTGCGTTAATCGATG | |
| | | CTAGTATTCAAAGCGAACACTTGCGT | |
| | | CCCGAATGGTTGGCAAGCGAAACCG | |
| | | GAATGAGTGTACGCTCTTTATACCGT | |
| | | CTGTTTGCCGATCAAGGGCTGGTAG | |
| | | TTGCCCAGTACATCAAGAATCGCCG | |
| | | TCTTGACTTGTGTGCCCAAGCTCTTC | |
| | | AGAATGTGCACGATGACGAGAAATT | |
| | | AGCCGGTATTGGTTATCGTTGGGGA | |
| | | TTTTCTGACCATTCTCACTTCAGTAC | |
| | | TGCTTTCAAGCAGCGTTTCGGAGTT | |
| | | ACGCCCGGGCAGTACCGTAAACGCT | |
| | | GTCGTTAA | |
| 130 | PtynA-KP/KA | tatgaacgtctggcaaaactgacaaacaagctggc | This work |
| | | aaaagcaaacaaacccctttgcatcagtactgataa | |
| | | tgtgaacctgactaaaccgcccacagagcgcggttg | |
| | | ctaacaagaacacaacagaaagaggggttaata | |

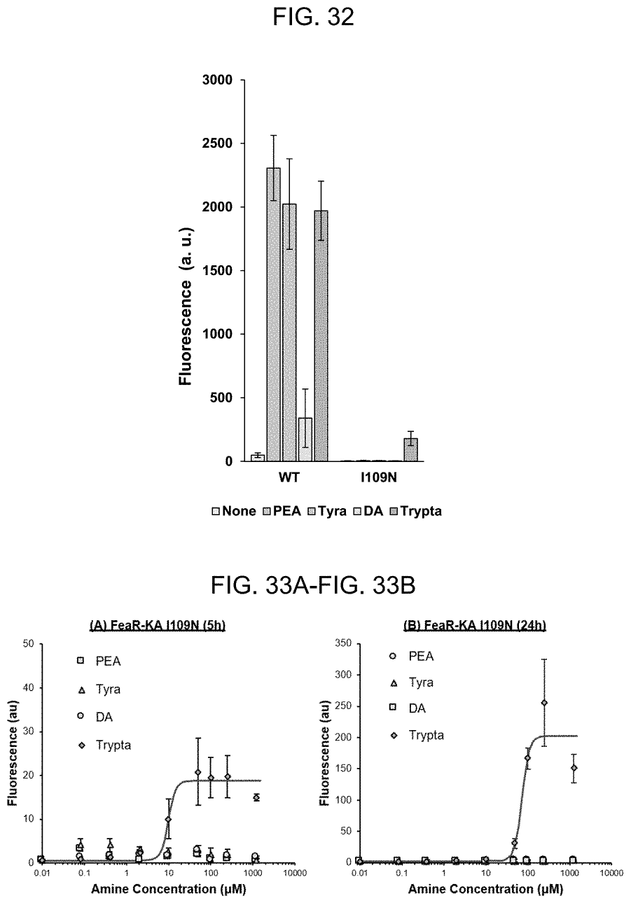

Next, significant enhancement of fluorescence intensity induced by Tryptamine was shown in the engineered sensor systems containing FeaR-KA (see e.g., FIG. 31). Moreover, a Tryptamine specific variant I109N was discovered using a sensor system with PtynA-MG, TynA-KP and FeaR-KA as the wild type (VVT) for further directed evolution (see e.g., FIG. 32). The Tryptamine specific variant I109N shows induction of fluorescence in response to tryptamine but not dopamine (DA), phenylethylamine (PEA), or tyramine (Tyra) (see e.g., FIG. 32). Transfer curves for the tryptamine-specific FeaR-KA I109N sensor after 5 or 24 hours of induction further illustrate the specificity of the sensor for tryptamine (see e.g., FIG. 33A-FIG. 33B).

What is claimed is:

1. An engineered molecular sensor comprising:
an engineered regulator protein or enzyme (e.g., TrpR, TyrR, TynA, FeaR) comprising a ligand-protein binding site;
a reporter (comprising a signaling moiety, e.g., GFP); and
a promoter (e.g., PtyrP) capable of inducing or repressing the reporter in the presence or absence of a target aromatic compound bound to the engineered molecular sensor,
wherein,
the engineered regulator protein or the ligand-protein binding site or a sequence encoding the engineered regulator protein or the ligand-protein binding site of the engineered regulator protein is at least about 80% identical to a WT regulator protein or a WT regulator ligand-protein binding site; or
a sequence encoding the engineered regulator protein or ligand binding site thereof is at least about 80% identical to a sequence encoding the WT regulator protein or the WT regulator ligand-protein binding site; or
functional fragment thereof, the engineered regulator protein having ligand binding activity; and
wherein the engineered molecular sensor is optionally genomically integrated into a microorganism, optionally selected from a probiotic or is a purified cell-free sensor.

2. The engineered molecular sensor of claim 1, wherein the engineered regulator protein or enzyme is an engineered TrpR, TyrR, TynA, or FeaR protein.

3. The engineered molecular sensor of claim 1, wherein
(a) the engineered regulator protein is an engineered TyrR and is an aromatic amino acid-specific sensor, wherein the aromatic amino acid-specific sensor is a phenylalanine (Phe)- or a tyrosine (Tyr)-specific sensor;
(b) the engineered regulator protein or enzyme is an engineered FeaR or TynA and is an aromatic amine-specific sensor, wherein the aromatic amine-specific sensor is a dopamine (DA)-, phenylethylamine (PEA)-, tyramine (Tyra)-, or tryptamine (Trypta)-specific sensor; or
(c) the engineered regulator protein is an engineered TrpR and is a tryptophan (Trp)-, 5-hydroxytryptophan (5-HTP)-, or tryptamine (Trpta)-specific sensor.

4. The engineered molecular sensor of claim 1, wherein the engineered regulator protein or enzyme is an engineered TrpR, TyrR, TynA, or FeaR, and wherein the engineered TrpR, TyrR, TynA, or FeaR comprises at least one mutation to a ligand-protein binding site having specific amino acid binding activity.

5. The engineered molecular sensor of claim 3, wherein:
PEA induces reporter expression;
Phe induces reporter expression; or
Tyr represses or induces reporter expression.

6. The engineered molecular sensor of claim 3, wherein: carboxylic acids 3,4-dihydroxyphenylacetic acid (DOPAC), phenylacetic acid (PAA), 4-hydroxyphenylacetic acid (HPPA), or indole-3-acetic acid (IAA) do not induce reporter expression or wherein IAA induces reporter expression.

7. The engineered molecular sensor of claim 1, wherein the engineered regulator protein is an engineered TyrR and the engineered TyrR comprises one or more of the following mutations:
E274Q+T14V mutations in TyrR inducing reporter expression in the presence of Phe;
E274Q+D103S mutations in TyrR inducing reporter expression in the presence of Phe; or
an R10F mutation in TyrR repressing reporter expression in the presence of Tyr,
wherein TyrR protein sequence is at least about 80% identical to SEQ ID NO: 124 or TyrR ligand binding site is at least about 80% identical to residues 7-274 of SEQ ID NO: 124;
a polypeptide encoded by SEQ ID NO: 11 or the WT ligand binding site sequence; or
functional fragment or conservative substitution thereof of TyrR having ligand binding functional activity.

8. A TyrR-based selective or specific sensor specifically detecting phenylalanine (Phe) or tyrosine (Tyr) comprising:
a TyrR or functional mutant or variant thereof; and
a reporter gene (comprising a signaling moiety, e.g., GFP) operably linked to an inducible promoter (e.g., PtyrP promoter).

9. The TyrR-based sensor of claim 8, wherein the inducible promoter comprises an activating response to Phe or a repressing response to Tyr or both Phe and Tyr.

10. The TyrR-based sensor of claim 8, wherein TyrR is selected from:
wild type (VVT) TyrR (SEQ ID NO: 124) or having at least about 80% identity to WT TyrR (SEQ ID NO: 124) and the reporter gene overexpresses if Phe is present;
TyrR or TyrR having at least about 80% identity to TyrR comprising the mutation E274Q and the reporter gene overexpresses in the presence of Phe and Phe+Tyr;
TyrR or TyrR having at least about 80% identity to TyrR comprising the mutation E274Q+T14V and the reporter gene overexpresses in the presence of Phe and Phe+Tyr and the reporter gene does not overexpress with Tyr in the absence of Phe;
TyrR or TyrR having at least about 80% identity to TyrR comprising the mutation E274Q+D103S and the reporter gene overexpresses in the presence of Phe and Phe+Tyr and the reporter gene does not overexpress with Tyr in the absence of Phe; or
TyrR or TyrR having at least about 80% identity to TyrR comprising the mutation R10F and the reporter gene is repressed with Tyr or Tyr+Phe and the reporter gene does not overexpress in the absence of Tyr.

11. The TyrR-based sensor of claim 8, wherein, the sensor is a target aromatic compound-inducible sensor or target aromatic compound-repressible sensor selected from:
a Phe-inducible TyrR system (e.g., E274Q, E274Q+T14V, E274Q+D103H, E274Q+D103S), or
Tyr-repressible TyrR system (e.g., R10F).

12. The TyrR-based sensor of claim 8, for use to kinetically diagnose or treat disorders that cause Phe-dysregulation without interference from intestinal Tyr.

13. The TyrR-based sensor of claim 8, comprising E274Q+T14V or E274Q+D103S variants of TyrR and wherein the TyrR-based sensor is sensitive to Phe in the presence or absence of Tyr and does not respond to Tyr alone.

14. The TyrR-based sensor of claim 8, comprising R10F variant of TyrR and wherein the TyrR-based sensor exhibits about a 12-fold repression in the presence of Tyr independent of the presence of Phe.

15. The TyrR-based sensor of claim 8, wherein the TyrR sensor has no significant response to Phe alone.

16. The TyrR-based sensor of claim 8, wherein a significant response is: under about 4000 au, under about 3000 au, or preferably at or under about 2000 au for Phe-inducible sensor; or
above about 2000 au or preferably at or above 2000 au for a Phe-repressible sensor.

17. The engineered molecular sensor of claim 1, wherein the engineered regulator protein or enzyme comprises an engineered FeaR or TynA or both and the engineered FeaR or TynA comprises one or more of the following mutations:
a G494S mutation in TynA inducing reporter expression in the presence of PEA;
a G494S mutation in TynA and A81T mutation in FeaR (e.g., G494S*) inducing reporter expression in the presence of PEA;
a G494S mutation in TynA and A81S mutation in FeaR inducing reporter expression in the presence of PEA;
a A81T or A81S mutation in FeaR inducing reporter expression in the presence of PEA and Tyra;
a A81T mutation in FeaR inducing reporter expression in the presence of PEA;
an A81T mutation in FeaR and S414M mutation in TynA inducing reporter expression in the presence of PEA;
an A81T mutation in FeaR and G415H mutation in TynA inducing reporter expression in the presence of PEA;
an A81L, A81P, A81I, or A81N mutation in FeaR inducing reporter expression in the presence of PEA;
a S414M or G415H mutation in TynA inducing reporter expression in the presence of Tyra;
a G415H mutation in TynA inducing reporter expression in the presence of Tyra;
a G415H mutation in TynA inducing reporter expression in the presence of Tyra and PEA;
a M83Y mutation in FeaR inducing reporter expression in the presence of PEA;
a M83N mutation in FeaR inducing reporter expression in the presence of Tyra;
a Fear-KA mutant inducing reporter expression in the presence of Trypta;
a tynA-KA or tynA-KP inducing reporter expression in the presence of Trypta;
a I109N tynA-KA inducing reporter inducing expression in the presence of Trypta;
a I109N FeaR-KA inducing reporter inducing expression in the presence of Trypta;
a Q76, Q116, L108(T), W110(S), or W110(C) FeaR mutation;
a D413 or Y496 TynA mutation; or
PtynA-MG, TynA-KP, or FeaR-KA inducing reporter inducing expression in the presence of Trypta and does not induce expression in the presence of dopamine (DA), phenylethylamine (PEA), or tyramine (Tyra),
wherein the FeaR or TynA protein sequence or ligand binding site of FeaR or TynA is at least about 80% identical to a polypeptide encoded by the WT FeaR sequence of SEQ ID NO: 36 or WT TynA sequence of SEQ ID NO: 32 or WT ligand binding site or functional fragment or conservative substitution thereof and has ligand-binding activity.

18. A selective sensor for specifically detecting target aromatic compounds (e.g., Phe, Tyr, PEA, Tyra) comprising:
a molecular sensor according to claim 1 or an engineered microorganism (e.g., an E. coli strain) capable of expressing native or non-native FeaR and TynA (or functional fragments, conservative substitutions, mutants, or variants thereof); and
a promoter-reporter system comprising a reporter gene under the control of a promoter capable of being induced or repressed by one or more target aromatic compounds (e.g., Phe, Tyr, PEA, Tyra) or enzymatic-reaction product thereof (e.g., aromatic aldehyde, aromatic carboxylic acid) and producing an output response (e.g., increased expression or repression).

19. The selective sensor of claim 18, wherein the output response is a repression of the reporter (e.g., signaling or detection moiety, such as GFP) expression.

20. The selective sensor of claim 18, wherein the output response is an overexpression of the reporter (e.g., signaling or detection moiety, such as GFP).

21. The selective sensor of claim 18, further comprising one or more enzymes or transcription factors.

22. A selective sensor for specifically detecting aromatic amines (e.g., PEA, Tyra, DA, Trypta) or aromatic aldehydes thereof comprising:
a TynA and FeaR protein (or a functional mutant or variant thereof); and
a reporter gene (comprising a signaling moiety, e.g., GFP) operably linked to an inducible promoter (e.g., PtynA promoter).

23. The selective sensor of claim 22, wherein the selective sensor induces expression of PtynA in response to aromatic aldehydes, but not aromatic amines.

24. The selective sensor of claim 22, wherein the TynA protein or mutant variant thereof is selective to a specific aromatic amine.

25. The selective sensor of claim 22, wherein TynA converts periplasmic amines (e.g., PEA, Tyra, DA, Trypta) to aldehydes which are imported into the cytoplasm; and in the cytoplasm, FeaR induces expression from the PtynA promoter expressing a reporter gene (e.g., detectable signal moiety) when in the presence of aldehydes.

26. The selective sensor of claim 22, wherein the selective sensor is selective to PEA and Tyra.

27. The selective sensor of claim 22, wherein either TynA or FeaR or both are engineered for selectivity.

28. The selective sensor of claim 22, wherein the sensor comprises G494S mutation in TynA and optionally A81T mutation in the FeaR protein (i.e., double mutant sensor (G494S*)).

29. The selective sensor of claim 22, wherein the selective sensor is a Tyra-specific variant, comprises G415H mutation in TynA.

30. The selective sensor of claim 22, wherein G415H mutation in TynA induces expression of a reporter by about 200-fold and/or about 79-fold in response to Tyra and PEA, respectively, with minimal response to DA and Trypta.

31. The selective sensor of claim 22, wherein
A81T FeaR mutation with WT TynA is PEA and Tyra non-selective;
S414M TynA and WT FeaR is slightly Tyra selective;
S141M TynA and A81T FeaR is PEA selective;
G415H TynA and WT FeaR is Tyra selective; or
G415H TynA and A81T FeaR is PEA selective.

32. The selective sensor of claim 22, wherein FeaR A81L, A81P, A81I, and A81N are PEA-specific variants.

33. The selective sensor of claim 22, wherein
Tyra-selective sensors (S414M or G415H TynA) can be transformed into PEA-specific sensors by introducing A81T FeaR;
sensor sensitivity (G494S TynA) can be improved by A81T or A81S FeaR mutations; or
PEA-Tyra selective sensors (A81T or A81S FeaR) can become PEA-specific sensors when combined with G494S TynA.

34. The engineered molecular sensor of claim 1, wherein the engineered regulator protein is an engineered TrpR and the engineered TrpR comprises one or more of the following mutations:
the TrpR variant is O_{D} or O₁, repressing reporter expression in the presence of Trp and 5-HTP, but not Trypta,
wherein the protein sequence or ligand binding site sequence of TrpR is at least about 80% identical to a polypeptide encoded by the WT TrpR sequence of SEQ ID NO: 113 or WT ligand binding site sequence or functional fragment or conservative substitution thereof and has ligand-binding activity.

35. The engineered molecular sensor of claim 1, wherein
the reference nucleotide sequence encoding TrpR is SEQ ID NO: 113 and the reference amino acid sequence for TrpR is SEQ ID NO: 122;
the reference nucleotide sequence encoding TyrR is SEQ ID NO: 11 and the reference amino acid sequence for TyrR is SEQ ID NO: 124;
the reference nucleotide sequence encoding FeaR is SEQ ID NO: 36 and the reference amino acid sequence for FeaR is SEQ ID NO: 125; or
the reference nucleotide sequence encoding TynA is SEQ ID NO: 32 and the reference amino acid sequence for TynA is SEQ ID NO: 123.

36. The engineered molecular sensor of claim 1, wherein the engineered regulator protein or enzyme (e.g., TyrR, FeaR, TynA, or TrpR) or the ligand binding site of the engineered regulator protein or enzyme comprises
at least 80% identity to a polypeptide encoded by SEQ ID NO: 32, SEQ ID NO: 36, SEQ ID NO: 11, or SEQ ID NO: 113;
at least 80% identity to SEQ ID NO: 122 (TrpR WT), SEQ ID NO: 123 (tynA WT), SEQ ID NO: 124 (tyrR WT), or SEQ ID NO: 125 (fear WT); or
at least 80% identity to D413 to Y496 of SEQ ID NO: 123, C7 to E274 of SEQ ID NO: 124, W12 to S118 of SEQ ID NO: 125, A81 to W110 of SEQ ID NO: 125, Q76 to Q116 of SEQ ID NO: 125, K72 to T83 of SEQ ID NO: 122, or a functional fragment or conservative substitution thereof.

37. The engineered molecular sensor of claim 1, wherein
the K_{A} value of the engineered regulator protein is less than the K_{A} value of the wild type regulator protein in the presence of a target aromatic compound;
the K_{A} value of the engineered TyrR sensor is between about 0.05 mM and about 0.3 mM optionally in human intestines, serum, or urine; or
the K_{A} value of the engineered TynA-FeaR is between about 0.001 mM and about 0.1 mM optionally in plasma or food.

38. The engineered molecular sensor of claim 1, wherein the presence of a target aromatic compound induces or represses reporter gene expression of the engineered regulator protein or enzyme compared to the wild type regulator protein or enzyme.

39. The engineered molecular sensor of claim 1, wherein the engineered regulator protein or enzyme has a selectivity, induction, or repression response that is greater than wild type.

40. The engineered molecular sensor of claim 1, wherein the regulator protein or enzyme or the regulator protein or enzyme binding site is modified to increase selectivity.

41. The engineered molecular sensor of claim 1, wherein the engineered molecular sensor is a ligand-specific biosensor for phenylalanine, tyrosine, phenylethylamine, or tyramine.

42. The engineered molecular sensor of claim 1, wherein the engineered molecular sensor is directly transferred into probiotic organisms or purified for cell-free sensor application.

43. A selective sensor for specifically detecting target aromatic compounds (e.g., Phe, Tyr) comprising:
an engineered microorganism (e.g., an E. coli strain) comprising the engineered molecular sensor according to claim 1 capable of expressing native or non-native TrpR (or functional mutants or variants thereof); and
a promoter-reporter system comprising a reporter gene (e.g., GFP) under the control of a promoter (e.g., Ptrp) capable of being induced or repressed by one or more target aromatic compounds and producing an output response (e.g., increased expression or repression of the reporter gene).

44. The selective sensor of claim 43, wherein the TrpR is a TrpR variant O_{D} or O₁, each with a synthetic Ptrp promoter, the selective sensor having strong repression in the presence of Trp and 5-HTP, but not Trypta.

45. The selective sensor of claim 43, wherein a genomic copy of wild-type trpR is knocked out from the engineered microorganism selected from an E. coli strain (e.g., EcN).

46. The selective sensor of claim 43, wherein the WT TrpR system has strong repression of GFP expression with fold repressions of 120-fold, 20-fold, and 7-fold in response to Trp, 5-HTP, and Trpta, respectively.

47. The selective sensor of claim 43, wherein the engineered TrpR variants maintain strong repression in the presence of Trp and 5-HTP, but not Trypta.

48. The selective sensor of claim 44, wherein the O_{D} variant demonstrates about 5-fold and about 7-fold repression in response to Trp and 5-HTP, respectively.

49. The selective sensor of claim 44, wherein the O₁ variant demonstrates about 60-fold and about 15-fold repression in response to Trp and 5-HTP, respectively.

50. The selective sensor of claim 43, wherein the target aromatic compounds are one or more of tryptophan (Trp), 5-hydroxytryptophan (5-HTP), or tryptamine (Trypta).

51. An artificial DNA construct comprising, as operably associated components in the 5′ to 3′ direction of transcription:
(a) a promoter functional in a microorganism (e.g., transgenic microorganism, wild type microorganism);
(b) a first polynucleotide comprising a nucleotide sequence encoding (i) a first polypeptide having TrpR activity; (ii) a second polypeptide having TyrR activity; or (iii) a first polypeptide having FeaR activity and a second polynucleotide comprising a nucleotide sequence encoding a second polypeptide having TynA activity;
(c) a reporter gene (e.g., GFP); and
(d) a transcriptional termination sequence;
wherein,
the microorganism is capable of expressing native or non-native (i) TrpR; (ii) TyrR; or (iii) FeaR and TynA (or functional mutants or variants thereof); and
the microorganism specifically expresses or represses reporter gene expression compared to a microorganism not comprising the artificial DNA construct in the presence or absence of aromatic compounds.

52. A microbial sensor selected from an engineered wild type or transgenic microorganism transformed with the artificial DNA construct of claim 51.

53. The microbial sensor of claim 52, wherein the wild type or transgenic microorganism is selected from Escherichia coli Nissle 1917 (EcN), DH10B, or E. coli MG1655.

54. The microbial sensor of claim 52, wherein the selective sensor is selective for an aromatic compound and the aromatic compound is an aromatic amino acid selected from phenylalanine (Phe), tyrosine (Tyr), and tryptophan (Trp), and combinations thereof.

55. The microbial sensor of claim 52, wherein the selective sensor is selective for an aromatic compound and the aromatic compound is an aromatic amine neurochemical selected from dopamine (DA), phenylethylamine (PEA), tyramine (Tyra), tryptamine (Trypta), serotonin, epinephrine, or norepinephrine.

56. The microbial sensor of claim 52, wherein a TrpR-based sensor is selective for Trp; a TyrR-based sensor is selective for Phe and Tyr; or a TynA-FeaR sensor system is selective for aromatic amines.

57. A ligand-specific sense-and-respond system, comprising purified sensors or engineered proteins or probiotics for specific sensing of aromatic compounds (e.g., amino acids, aromatic amines, aromatic neurochemicals) comprising:
providing a orthogonal DNA-TF binding system with accompanying selectivity changes;
changing ligand-TF binding specificity by leveraging differential multimerization patterns of TyrR without affecting DNA-TF binding interaction; or
a “dual-control knob” strategy to improve the specificity and sensitivity of substrate-enzyme and ligand-TF interaction while maintaining DNA-TF binding interaction.

58. The ligand-specific sense-and-respond system of claim 57, wherein target ligands are structurally similar and ligand-protein binding controls downstream functions such as reporter gene expression.

59. The ligand-specific sense-and-respond system of claim 57, comprising an engineered microorganism.

60. A method of using the engineered molecular sensor of claim 1, comprising obtaining or having obtained a biological sample from a subject and contacting the biological sample with the engineered molecular sensor.

61. The method of claim 60, wherein the subject has an aromatic compound-associated disease, disorder, or condition.

62. The method of claim 60, wherein elevated levels of Phe detected by the sensor indicate the subject has phenylketonuria.

63. The method of claim 60, wherein elevated levels of Tyr detected by the sensor indicate the subject has type 2 tyrosinemia.

64. The method of claim 60, wherein elevated levels of PEA detected by the sensor indicate the subject has a psychological disorder.

65. The method of claim 60, wherein the presence of Tyra detected by the sensor indicates catecholamine release and an increase in blood pressure.

66. The method of claim 60, wherein the presence of Trypta detected by the sensor causes serotonin release and stimulation of gastrointestinal motility.

67. A method of using the engineered molecular sensor of claim 1, comprising monitoring food quality or diagnosing or treating metabolic, digestive, or neurological disorders.

68. The method of claim 67, wherein the presence of PEA, Tyra, or Trypta in food detected by the sensor indicates microbial contamination.

69. The engineered molecular sensor of claim 1, wherein the sensor dynamically identifies microbial contamination in consumable products, manages various debilitating neurological disorders, or normalizes dysregulated metabolites associated with metabolic disorders.

70. The engineered molecular sensor of claim 1, wherein the sensor recognizes or is selective for aromatic metabolites associated with various metabolic or neurological disorders or medical conditions.

71. The engineered molecular sensor of claim 70, wherein the aromatic compounds are selected from phenylalanine (Phe) or tyrosine (Tyr).

72. The engineered molecular sensor of claim 70, wherein the aromatic compounds are neurochemicals.

73. The engineered molecular sensor of claim 72, wherein the neurochemicals are selected from aromatic neurotransmitters or neuromodulators.

74. The engineered molecular sensor of claim 72, wherein the neurochemicals are selected from dopamine (DA), phenylethylamine (PEA), tyramine (Tyra), tryptamine (Trypta), serotonin, epinephrine, or norepinephrine.

75. The method of claim 60, wherein the subject has or is suspected of having a medical condition associated with elevation, presence, or absence of aromatic compounds.

76. The engineered molecular sensor of claim 1, wherein the sensor differentiates metabolites with divergent functions even having structural similarity.

77. The engineered molecular sensor of claim 1, wherein the sensor modulates the specificity of ligand-protein binding while maintaining protein-DNA interactions and downstream gene expression control.

78. A method of using the engineered molecular sensor of claim 1, the method comprising monitoring food quality, diagnosing or treating metabolic, digestive, or neurological disorders in probiotics or ex vivo wearable, paper-based or cell-free systems, or dynamically regulating enzymatic pathways for microbial metabolic engineering using the engineered molecular sensor.

79. A method of protein engineering (e.g., a regulator protein or enzyme) comprising:
mutagenizing specific amino acids in and around a ligand-binding site of a protein or enzyme (e.g., TrpR, TyrR, FeaR, TynA), wherein the mutagenizing enables changes in ligand-protein binding specificity while maintaining protein-DNA interaction and thus downstream gene expression control; and
linking ligand-protein binding to output response (e.g., promoter-reporter gene system).
